(19) United States
(12) Patent Application Publication
(10) Pub. No.: US 2005/0239873 A1
(43) Pub. Date: Oct. 27, 2005

(54) 2 Methoxy antimycin a derivatives and methods of use

(75) Inventors: David M. Hockenbery, Seattle, WA (US); Julian A. Simon, Seattle, WA (US); Shie-Pon Tzung, Issaquah, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Fred Hutchinson Cancer Research Center, Seattle, WA (US)

(21) Appl. No.: 11/036,645

(22) Filed: Jan. 14, 2005

Related U.S. Application Data
(63) Continuation-in-part of application No. 10/069,431, filed on Jul. 30, 2002.
(60) Provisional application No. 60/149,968, filed on Aug. 20, 1999.
(60) Provisional application No. 60/644,349, filed on Jan. 14, 2005.

(51) Int. Cl. A61K 31/365

(52) U.S. Cl. 514/450; 549/267

(57) ABSTRACT

Disclosed are 2-methoxy antimycin derivatives or analogs that modulate apoptosis by binding to the hydrophobic groove of a Bcl-2 family member protein (e.g., Bcl-2 or BCl-x_{L}). The 2-methoxy antimycin derivatives or analogs are used in disclosed methods for treating apoptosis-associated diseases such as, for example, neoplastic disease (e.g., cancer) or other proliferative diseases associated with the over-expression of a Bcl-2 family member protein.

### RELATED APPLICATIONS

This application is a continuation-in-part of U.S. patent application Ser. No. 10/069,431, which is a United States National Phase Application of International Patent Application Serial number PCT/US00/22891, filed Aug. 18, 2000, which claims the benefit of United States Provisional Patent Application 60/149,968, filed Aug. 20, 1999, all of which are incorporated herein by reference in their entirety. This application also claims benefit of the United States Provisional Application 60/XXX,XXX, filed Jan. 14, 2005 and entitled “Methods for Identifying Agents that Modulate Apoptosis in Cells that Over-express a Bcl-2 Family Member Protein” (Attorney Docket No. 14538A-008000US), incorporated herein in its entirety.

### STATEMENT AS TO RIGHTS TO INVENTIONS MADE UNDER FEDERALLY SPONSORED RESEARCH AND DEVELOPMENT

This work was supported by grants from the National Institutes of Health: Pilot Award from Cancer Center Support Grant 5P30CA015704-3 and U01 Cooperative Agreement 1U01CA91310. The U.S. government may have certain rights in the invention.

### BACKGROUND OF THE INVENTION

Members of the evolutionarily conserved Bcl-2 family are important regulators of apoptotic cell death and survival. The proteins Bcl-2, Bcl-x_{L}, Bcl-w, A1 and Mc1-1 are death antagonists while Bax, Bak, Bad, Bcl-xs, Bid, and Bik are death agonists (Kroemer et al., Nature Med. 6:614-620, 1997). Bcl-2 family member proteins are predominantly localized in the outer mitochondrial membrane, but are also found in the nuclear membrane and endoplasmic reticulum (Kroemer et al., supra).

Among Bcl-2 family member proteins, there are several conserved amino acid motifs designated, BH1 through BH4. The pro-apoptotic members of the family, Bax and Bad, contain a BH3 domain that is sufficient to induce cell death (Chittenden et al., EMBO J. 14:5589-5596, 1995; Hunter et al., J. Biol. Chem. 271:8521-8524, 1996). Interestingly, the BH3 domain is conserved in the anti-apoptotic proteins Bcl-2 and BC1-X_{L}. Recently, it was reported that cleavage of BC1-x_{L} and Bcl-2 in the loop domain removes the N-terminal BH4 domain and converts BC1-x_{L} and Bcl-2 into a potent pro-death molecule (Cheng et al., Science 278:1966-1968, 1997; Clem et al., Proc. Nat. Acad. Sci. USA 95:554-559, 1998).

NMR structure analysis of a complex between Bcl-x_{L} and a 16 residue peptide encompassing the Bak BH3 domain demonstrated that the BH3 peptide, in an amphipathic alpha-helical configuration, binds with high affinity to the hydrophobic pocket created by the BH1, BH2 and BH3 domains of BC1-x_{L} (Sattler et al., Science 275:983-986, 1997). Leucine at position 1 of the BH3 domain core and aspartic acid at position 6 are believed to be critical residues for both heterodimerization and apoptosis induction. In further support of this conclusion, a number of “BH3 only” death promoters have been identified which have no similarity to Bcl-2 beyond their BH3 domain homology (Kelekar et al., Trends Cell Biol. 8:324-330, 1998). These include Bik, Bim, Hrk, Bad, Blk, and Bid, which cannot homodimerize, but rely on binding to anti-apoptotic proteins such as Bcl-2 to induce cell death.

The exact mechanisms by which Bcl-2 prevents apoptosis remain elusive. In light of the importance of mitochondria in apoptosis and the mitochondrial location of Bcl-2, it appears that one major site where Bcl-2 interrupts apoptotic signals is at the level of mitochondria. Mitochondria play a central role in mediating apoptosis in a number of apoptotic models (Kroemer et al., Immunol. Today 18:44-51, 1997; Zamzami et al., J. Exp. Med. 183:1533-1544, 1996; Zamzami et al., J. Exp. Med. 182:367-377, 1995). Cells induced to undergo apoptosis show an early disruption of mitochondrial transmembrane potential (ΔΨₘ) preceding other changes of apoptosis, such as nuclear fragmentation and exposure of phosphatidylserine on the outer plasma membrane. Isolated mitochondria or released mitochondrial products induce nuclear apoptosis in a cell-free reconstituted system (Liu et al., Cell 86:147-157, 1996; Newmeyer et al., Cell 79:353-364, 1994).

It has been shown that Bcl-2 inhibits apoptosis concomitant with mitochondrial permeability transition and by stabilizing ΔΨₘ (Zamzami et al., J. Exp. Med. 183:1533-1544, 1996). In the absence of Bcl-2, apoptogenic factors, such as cytochrome c and apoptosis inducing factor (AIF), are released from mitochondria in response to apoptotic triggers (Susin et al, J. Exp. Med. 184:1331-1341, 1996; Kluck et al., Science 275:1132-1136, 1997). This release in turn leads to sequential caspase activation and results in nuclear and membrane changes associated with apoptosis.

Bcl-2 family members display a distinct tissue-specific expression. In adult human liver, Bcl-2 expression is confined to bile duct cells (Charlotte et al., Am. J. Pathol. 144:460-465, 1994) and is absent in both normal and malignant hepatocytes. In contrast, expression of BCl-x_{L} RNA and protein can be detected in adult quiescent hepatocytes and increases by 4 to 5 fold during the G₁ phase of regenerating hepatocytes (Tzung et al., Am. J. Pathol. 150:1985-1995, 1997). Increased BCl-x_{L} expression is also observed in hepatoma cell lines, such as HepG2.

Some diseases are believed to be related to the down-regulation of apoptosis in the affected cells. For example, neoplasias may result, at least in part, from an apoptosis-resistant state in which cell proliferation signals inappropriately exceed cell death signals. Furthermore, some DNA viruses, such as Epstein-Barr virus, African swine fever virus and adenovirus, parasitize the host cellular machinery to drive their own replication and at the same time modulate apoptosis to repress cell death and allow the target cell to reproduce the virus. Moreover, certain diseases, such as lymphoproliferative conditions, cancer (including drug resistant cancer), arthritis, inflammation, autoimmune diseases, and the like, may result from a down regulation of cell death signals. In such diseases, it would be desirable to promote apoptotic mechanisms.

Most currently available chemotherapeutic agents target cellular DNA and induce apoptosis in tumor cells (Fisher et al., Cell 78:539-542, 1994). A decreased sensitivity to apoptosis induction has emerged as an important mode of drug resistance. In particular, over-expression of Bcl-2 and BCl-x_{L} confers resistance to multiple chemotherapeutic agents, including alkylating agents, antimetabolites, topoisomerase inhibitors, microtubule inhibitors and anti-tumor antibiotics, and may constitute a mechanism of clinical chemoresistance in certain tumors (Minn et al., Blood 86:1903-1910, 1995; Decaudin et al., Cancer Res. 57:62-67, 1997). Bcl-2/Bcl-x_{L}-directed therapies, using either anti-sense oligonucleotides or novel protein-targeted drugs, can increase cellular sensitivity to standard agents in vitro or, in some cases, kill cells as single agents (Jansen et al., Nat. Med. 4:232-234, 1998). Structure solutions for BCl-x_{L} and Bcl-2 have demonstrated the presence of a hydrophobic cleft at the surface of both proteins (Muchmore et al., Nature 381:335-341, 1996). Functional studies implicate this groove as a binding surface for heterodimenc partners, including the related pro-apoptotic proteins Bax and Bak, and as a regulatory domain for an intrinsic membrane pore function (Sattler et al., Science 275:983-986, 1997). Efforts to design small molecule inhibitors of Bcl-2/ BCl-x_{L} have thus focused on this structural feature.

Neither Bcl-2 nor Bcl-x_{L}, however, protects cells from every apoptotic inducer. For example, over-expression of Bcl-2 offers little protection against Thy-1-induced thymocyte death and Fas-induced apoptosis (Hueber et al., J. Exp. Med. 179:785-796, 1994; Memon et aL, J. Immunol. 15:4644-4652, 1995). At the mitochondrial level, Bcl-2 over-expressed in the outer mitochondrial membrane inhibits PT pore induction by t-butyl-hydroperoxide, protonophore and atractyloside, but not by calcium ions, diamide or caspase 1 (Zamzami et al., J. Exp. Med. 183:1533-1544, 1996; Susin et al., J. Exp. Med. 186:25-37, 1997). Thus, one class of mitochondrially-active agents may directly affect the mitochondrial apoptosis machinery while bypassing the site of Bcl-2 function and the protection offered by Bcl-2 family members. An agent of this type may potentially be useful in overcoming the multi-drug resistance imparted by Bcl-2 or BCl-x_{L} and are of great need in the art.

The antimycins constitute another class of mitochondrially-active agents. The antimycins generally comprise a N-formylamino salicylate moiety linked to a dilactone ring through an amide bond. The antimycins differ in the hydrophobic R groups attached to the dilactone ring opposite the amide bond. (See, e.g., Rieske, Pharm. Ther. 11:415-420, 1980.) For example, antimycin A₁ has a hexyl group at the R₁ position of the dilactone ring while antimycin A₃ has a butyl group at that position. Extensive literature has been published on the structure-activity relationship of the antimycins and their inhibition of cytochrome bc₁ (Miyoshi et al., Biochim. Biophys. Acta 1229:149-154, 1995; Tokutake et al., Biochim. Biophys. Acta 1142:262-268, 1993; Tokutake et al., Biochim. Biophys. Acta 1185:271-278, 1994). The published structure of cytochrome bc₁ complex with bound antimycin A₁ reveals that antimycin A₁ occupies a position in the Qi ubiquinone binding site on cytochrome b (Xia et al., Proc. Nat. Acad. Sci. USA 94:11399-11404, 1997). The antimycins generally inhibit mitochondrial respiration, which suggests that the differences in the hydrophobic R groups on the dilactone ring are not critical for cytochrome b binding. Mutagenesis and structure-activity studies of antimycin A demonstrate that the cytochrome bc₁-inhibitory activity is highly dependent on the N-formylamino salicylic acid moiety (Tokutake et al., 1994, supra). Methylation of the phenolic hydroxyl or modification of the N-formylamino group both significantly reduce the ability of certain antimycin A derivatives to bind to and inhibit cytochrome bc₁. Methylation of the phenolic hydroxyl diminishes inhibitory activity by 2.5 logs. Substitution of the formylamino group with acetylamino and propylamino groups at the 3-position reduce cytochrome bc₁ activity by 1.2 and 2.4 logs, respectively. Thus, the N-formylmino salicylate moiety is generally understood to be important for binding of the antimycins to cytochrome b.

Two antimycins, antimycin A₁ and A₃, have been discovered to inhibit the activity of the anti-apoptotic Bcl-2 family member proteins, Bcl-2 or BCl-x_{L}. Thus, these molecules or derivatives thereof are potentially useful compounds for the medical profession and patients suffering from proliferative disease and other diseases where apoptosis is inappropriately regulated. But the naturally obtained antimycins are toxic, however, because as discussed above, they also inhibit mitochondrial respiration. There is a critical need, therefore, for derivatives of the antimycins that are effective in inducing apoptosis in cells where apoptosis is inappropriately regulated while exhibiting reduced inhibition of mitochondrial respiration.

### SUMMARY OF THE INVENTION

The present invention is based on the discovery that the 2-methoxy derivatives and/or analogs of antimycins can inhibit the activity of anti-apoptotic Bcl-2 family member proteins, such as Bcl-2 or Bcl-x_{L}. The invention is further based on the discovery that mitochondrial respiratory inhibitory activity, of the antimycins, is separable from the inhibition of the Bcl-2 family member proteins and that the 2-methoxy derivatives and/or analogs of the anitimycins can induce apoptosis in cells that over-express an anti-apoptotic Bcl-2 family protein without inhibiting oxidative phosphorylation.

The present invention provides agents comprising 2-methoxy derivatives and/or analogs of antimycins that modulate apoptosis by binding to a Bcl-2 family member protein. These agents exhibit reduced binding to cytochrome B (or the cytochrome bcl complex, hereafter referred to as “cytochrome B”) as compared with antimycins found in nature. In one embodiment, the agent preferentially induces apoptosis in cells that over-express an anti-apoptotic Bcl-2 family member protein and is substantially non-toxic to cells that do not over-express the anti-apoptotic Bcl-2 family member protein. The agent typically inhibits the activity of an anti-apoptotic Bcl-2 family member protein by binding to the hydrophobic pocket formed by the BH1, BH2 and BH3 domains of the protein.

The agents comprises derivatives and/or of an antimycin, or a portion thereof, such as chemical modification of the N-formylamino salicylic acid moiety (e.g., salicylic acid or acetylsalicylic acid), and/or the dilactone moiety (i.e., the 4,9-dioxo-1,5-dioxanan-7-yl ester moiety). In a preferred embodiment, the antimycin derivative comprises at least two chemical modifications. The first modification that decreases the affinity of the antimycin derivative for cytochrome B comprises the methylation of the N-formyl amino group on the salicylic acid ring. The second modification increases the affinity of the antimycin derivative for a Bcl-2 family member protein.

In another aspect, the invention provides methods for modulating apoptosis in a cell. Such methods generally comprise administering an agent comprising a 2-methoxy derivative and/or analog of an antimycin to modulate apoptosis in the cell. In one embodiment, the agent preferentially induces apoptosis in a cell that over-expresses an anti-apoptotic Bcl-2 family member protein. The agent typically exhibits reduced binding affinity for cytochrome B and is substantially non-toxic to cells that do not over-express the anti-apoptotic Bcl-2 family member protein. The agent comprises a derivative and or analog, such as derivatives of the N-formylamino salicylic acid moiety (e.g., salicylic acid or acetylsalicylic acid,) or the dilactone moiety. In another embodiment, the method comprises administering the agent to inhibit the activity of the anti-apoptotic Bcl-2 family member protein by binding to the hydrophobic pocket formed by the BH1, BH2 and BH3 domains of the protein.

In another aspect, the invention provides pharmaceutical compositions comprising the agent, as well as the use of such pharmaceutical compositions, for treating a subject in which a cell over-expresses an anti-apoptotic Bcl-2 family member protein. Such compositions and methods are useful for treating apoptosis-associated diseases or conditions, such as drug-resistance. In a preferred embodiment, the compositions and use thereof preferentially induce apoptosis in cells that over-express the anti-apoptotic Bcl-2 family member protein. The agent typically exhibits reduced binding affinity for cytochrome B and is substantially non-toxic to cells that do not over-express the anti-apoptotic Bcl-2 family member protein. The compositions can further comprise any agent commonly used to treat cancer, such as for example, etoposide, melphalan, daunorubicin, paclitaxel, 5-flurouracil, cisplatin, and the like. Further, the compositions can be used in combination with other cancer treatment modalities such as surgery, radiation therapy, and the like.

The present invention further provides methods for assaying candidate compounds to identify agents that modulate the activity of a Bcl-2 family member protein. The methods generally comprise the steps of administering the candidate compound to a cell that over-expresses the Bcl-2 family member protein; administering the candidate compound to another cell that does not over-express the Bcl-2 family member protein; and determining whether the candidate compound modulates the activity of the Bcl-2 family member protein to produce a physiological change in the cell that over-expresses the Bcl-2 family member protein, but does not produce a substantial physiological change in the cell which does not over-express that protein. In a preferred embodiment, the Bcl-2 family member protein is anti-apoptotic. Cells that over-express the anti-apoptotic Bcl-2 family member protein, such as BCl-x_{L} or Bcl-2, are produced by, for example, transfection with a gene or cDNA fragment that encodes the protein. The gene and or cDNA can also encode various mutations of the BCl-x_{L} or Bcl-2 protein for compsrison by the below described methods to determine the difference in a measure of apoptosis with wild-type BCl-x_{L} or Bcl-2 for the selection of additional 2-methoxy derivatives and/or analogs of antimycin that can inhibit apoptosis of cells that overexpress BCl-x_{L}. The cells can be any mammalian cell and in a particular embodiment are murine liver cells. Physiological changes that are indicative of binding of the candidate compound to the Bcl-2 family member protein (e.g., in the hydrophobic pocket) include an affect on cell death, cell shrinkage, chromosome condensation and migration, mitochondria swelling, and/or disruption of mitochondrial transmembrane potential.

### BRIEF DESCRIPTION OF THE DRAWINGS

FIG. 1 depicts a general scheme for the chemical synthesis of antimycin A₃, and for the synthesis of derivatives and/or analogs of antimycins.

### DESCRIPTION OF THE SPECIFIC EMBODIMENTS

Definitions:

The term “apoptosis” refers to a regulated network of biochemical events which lead to a selective form of cell suicide, and is characterized by readily observable morphological and biochemical phenomena, such as the fragmentation of the deoxyribo-nucleic acid (DNA), condensation of the chromatin, which may or may not be associated with endonuclease activity, chromosome migration, margination in cell nuclei, the formation of apoptotic bodies, mitochondrial swelling, widening of the mitochondrial cristae, opening of the mitochondrial permeability transition pores and/or dissipation of the mitochondrial proton gradient and the like.

The term “antimycins” refers to the known antimycins, including, for example, the antimycins A_{0(a-d)}, A₁ₐ, A_{1b}, A₂, A₃, the aniline of A₃, A₄, A₅, A₆, kitamycin A and B, urauchimycin A and B, deisovaleryl blastomycin, and dehexyl-deisovaleryloxy antimycin A, and the like. The antimycins are generally represented by formula I, and have the absolute configuration [2R, 3R, 4S, 7S, 8R] (Kinosjita, Antibiotics 25:373, 1972):

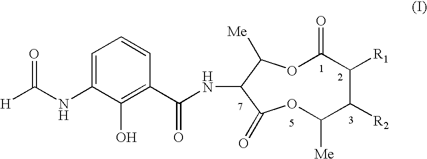

The groups at positions R₁ and R₂ vary as follows:

TABLE 1

| Name | R₁ | R₂ |
|---|---|---|
| antimycin A₀₍ₐ₎ | hexyl | hexanoic acid |
| antimycin A_{0(b)} | butyl | heptanoic acid |
| antimycin A_{0(c)} | octyl | butanoic acid |
| antimycin A_{0(d)} | heptyl | isovaleric acid |
| antimycin A_{1b} | hexyl | isovaleric acid |
| antimycin A₂ | hexyl | butanoic acid |
| antimycin A₃ | butyl | isovaleric acid |
| antimycin A₄ | butyl | butanoic acid |
| antimycin A₅ | ethyl | isovaleric acid |
| antimycin A₆ | ethyl | butanoic acid |
| kitamycin A | hexyl | hydroxyl |
| kitamycin B | isohexyl | hydroxyl |
| urauchimycin B | isohexyl | hydroxyl |
| deisovalerylblastomycin | butyl | hydroxyl |
| dehexyl-deisovalerylblastomycin | hydrogen | hydrogen |

The term “antimycin derivative or analog” refers to a chemical modification of an antimycin structure, by which one or more atoms of an antimycin are removed or substituted, or new atoms are added. “Antimycin derivative or analog” encompass both those compounds that can be made using antimycin itself as the starting molecule (e.g., isolating antimycin from a natural source and then changing the molecule) as well as compounds that are structurally related to antimycin but that are not synthesized directly from an antimycin molecule. An “antimycin derivative or analog” further includes portions of an antimycin as well as chemical modifications thereof, and chiral variants of an antimycin. In particular, a “2-methoxy antimycin derivative or analog” (“2-OMeA derivative”) of the present invention refers to an antimycin derivative or analog in which the phenolic hydroxyl group is methylated and R₁ and R₂ can vary as set forth herein. Particularly preferred 2-OMeA derivatives or analogs of the present invention are those derivatives that can inhibit the binding of Bcl-2 to BH3, or can kill cells that over express Bcl-2 and are substrantially non-toxic to those cells that do not overexpress Bcl-2.

The term “preferentially induce” apoptosis refers to at least a 5-fold greater stimulation of apoptosis, at a given concentration an agent, including a 2-methoxy antimycin derivative, in cells that over-express a Bcl-2 family member protein as compared with cells that do not over-express the Bcl-2 family member protein (e.g., a 5-fold lower LD₅₀ or IC₅₀).

The term “substantially non-toxic” refers to an agent including a 2-methoxyantimycin that induces apoptosis in at least about 50 percent of cells that over-express a Bcl-2 family member protein, but does not induce apoptosis in more than about 5%, more preferably less than 1%, of cells that do not over-express the Bcl-2 family member protein.

The term “Bcl-2 family member protein(s)” refers to an evolutionarily conserved family of proteins characterized by having one or more amino acid homology domains, BH1, BH2, BH3, and/or BH4. The Bcl-2 family member proteins include Bcl-2, BCl-x_{L}, Bcl-w, A1, Mcl-1, Bax, Bak, Bad, Bcl-xs, Bid and the like. The “Bcl-2 family member proteins” further include those proteins, or their biologically active fragments, that have at least 70%, preferably at least 80%, and more preferably at least 90% amino acid sequence identity with a Bcl-2 family member protein.

The term “anti-apoptotic Bcl-2 family member protein” refers to Bcl-2, BCl-x_{L}, Bcl-w, A1, Mcl-1, and other proteins characterized by having one or more amino acid homology domains, BH1, BH2, BH3, and/or BH4, and that promote cell survival by attenuating or inhibiting apoptosis. The “anti-apoptotic Bcl-2 family member proteins” further include those proteins, or their biologically active fragments, that have at least 70%, preferably at least 80%, and more preferably at least 90% amino acid sequence identity with an anti-apoptotic Bcl-2 family member protein.

The terms “identity” or “percent identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using either a PILEUP or BLAST sequence comparison algorithm (see, e.g., J. Mol. Evol. 35:351-360, 1987; Higgins and Sharp, CABIOS 5:151-153, 1989; Altschul et al., J. Mol. Biol. 215:403-410, 1990; Zhang et al., Nucleic Acid Res. 26:3986-3990, 1998; Altschul et al., Nucleic Acid Res. 25:3389-33402, 1997). Optimal alignment of sequences for comparison can be conducted, e.g., by the local homology algorithm of Smith and Waterman, Adv. Appl. Math. 2:482, 1981, by the homology alignment algorithm of Needleman and Wunsch, J. Mol. Biol. 48:443, 1970, by the search for similarity method of Pearson and Lipman, Proc. Nat. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see, generally, Ausubel et al., supra).

In the context of Bcl-2 family member proteins, “correspondence” of one polypeptide sequence to another sequence (e.g., regions, fragments, nucleotide or amino acid positions, or the like) is based on the convention of numbering according to nucleotide or amino acid position number, and then aligning the sequences in a manner that maximizes the number of nucleotides or amino acids that match at each position, as determined by visual inspection or by using a sequence comparison algorithm such as, for example, PILEUP (see, e.g., supra; Higgins & Sharp, supra) or BLAST (see, e.g., Altschul et al., supra; Zhang et al., supra; Altschul et al., supra). For example, a mutant Bcl-2 family member amino acid sequence having one or more amino acid substitutions, additions, or deletions as compared to the wild-type protein may correspond to a second Bcl-2 family member amino acid sequence (e.g., the wild-type sequence or a functionally equivalent variant thereof) according to the convention for numbering the second Bcl-2 family member sequence, whereby the mutant sequence is aligned with the second Bcl-2 family member sequence such that at least 50%, typically at least 60%, more typically at least 70%, preferably at least 80%, more preferably at least 90%, and even more preferably at least 95% of the amino acids in a given sequence of at least 20 consecutive amino acids are identical. Because not all positions with a given “corresponding region” need be identical, non-matching positions within a corresponding region are herein regarded as “corresponding positions.”

As used herein, a single amino acid substitution in one (“first”) mutant Bcl-2 family member protein “corresponds” to a single amino acid substitution in a second mutant Bcl-2 family member protein (e.g., Bcl-x_{L}) where the corresponding substituted amino acid positions of the first and second mutant proteins are identical.

In the context of Bcl-2 family member protein mutants, the phrase “no substantial effect on tertiary protein structure relative to the corresponding wild-type Bcl-2 family member protein” or “no substantial alteration of tertiary protein structure relative to the corresponding wild-type Bcl-2 family member protein” means that, when a Cα trace providing a position for each Cα carbon of the mutant protein is superimposed onto a Cα trace of the corresponding wild-type protein and an α carbon root mean square (RMS) difference root mean square deviation (RMSD) is calculated; i.e., the deviation of the mutant structure from that of the wild-type structure), the RMSD value is no more than about 1.0 Å when calculated using the same structural modeling method, typically no more than about 0.75 Å, even more typically no more than about 0.5 Å, preferably no more than about 0.35 Å, and even more preferably no more than about 0.25 Å.

The terms “biologically active” or “biological activity” refer to the ability of a molecule to modulate apoptosis, such as by binding to a Bcl-2 family member protein. A biologically active molecule can modulate apoptosis by causing a change in the mitochondrial protonmotive force gradient (see, e.g., Example 2); by causing a change in mitochondrial swelling or the morphological characteristics of mitochondria (see, e.g., Example 2); by affecting the release of a reporter molecule, such as, for example, rhodamine 123 or calcein, from mitochondria or vesicles (see, e.g., Examples 4 and 8) comprising a pore-forming anti-apoptotic Bcl-2 family member protein (see, e.g., Example 8); or by causing any other morphological change associated with apoptosis.

The terms “therapeutically useful” and “therapeutically effective” refer to an amount of an agent that effectively modulates the apoptotic state of an individual cell such that apoptosis is induced and/or the inappropriately regulated cell death cycle in the cell returns to a normal state.

The terms “diagnostically useful” and “diagnostically effective” refer to an agent (e.g., an antimycin derivative) for detecting the induction or inhibition of apoptosis in a subject. These terms further include molecules useful for detecting diseases associated with apoptosis, or the susceptibility to such diseases, and for detecting over-expression or under-expression of a Bcl-2 family member protein.

The terms “over-expression” and “under-expression” refer to an increase or decrease, respectively, in the levels of a Bcl-2 family member protein in a cell relative to the level of such a protein found in the same cell or a closely related non-malignant cell under normal physiological conditions.

The term “apoptosis-associated disease” includes diseases, disorders, and conditions that are linked to an increased or decreased state of apoptosis in at least some of the cells of a subject. Such diseases include neoplastic disease (e.g., cancer and other proliferative diseases), tumor formation, arthritis, inflammation, autoimmune disease, human immunodeficiency virus (HIV) immunodeficiency syndrome, neurodegenerative diseases, myelodysplastic syndromes (such as aplastic anemia), ischaemic syndromes (such as myocardial infarction), liver diseases which are induced by toxins (such as alcohol), alopecia, damage to the skin due to UV light, lichen planus, atrophy of the skin, cataract, and graft rejections. Neurodegenerative diseases include Alzheimer's disease, Parkinson's disease, amyotrophic lateral sclerosis, and other diseases linked to degeneration of the brain, such as Creutzfeldt-Jakob disease. Apoptosis-associated diseases further include drug resistance associated with increased or decreased levels of an anti-apoptotic Bcl-2 family member protein as well as multiple chemotherapeutic drug resistance.

2-Methoxy Antimycin Derivatives:

The present invention provides 2-methoxy antimycin (2-OMe antimycin, 2-OMeA) derivatives that modulate apoptosis of a cell by binding to a Bcl-2 family member protein. In addition, the 2-methoxy antimycin derivatives typically exhibit reduced binding affinity to cytochrome B relative to non-derivatized antimycin making these compounds substantially nontoxic to cells that do not over express a Bcl-2 family membrane protein. The 2-methoxy antimycin derivatives preferentially induce apoptosis in cells that over-express a Bcl-2 family member protein.

In one embodiment, the 2-methoxy antimycin derivative is of the following Formula (II): 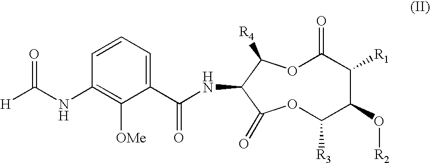
where each of positions R₁-R₄ can be independently modified, with the proviso that R₂ is an (C₁-C₈) acyl group, and further with the proviso that the antimycin derivative is not 2-methoxy antimycin A₃. For example, each of R₁, R₃, and R₄ can independently be hydrogen, a C₁-C₁₀ (e.g., C₁-C₈) linear or branched alkane (e.g., methyl, ethyl, butyl, isobutyl, pentyl, isopentyl, and the like), hydroxyl, a C₁-C₁₀ (e.g., C₁-C₈) hydroxyalkane (e.g., hydroxymethyl, hydroxyethyl, hydroxypropyl, and the like), amino, an amino halogen salt (e.g., amino chloride, amino bromide or amino fluoride), a C₁-C₁₀ (e.g., C₁-C₈) di- or tri-alkylamine (e.g., methyl amine, dimethyl amine, ethyl amine, diethyl amine, and the like), a C₁-C₁₀ (e.g., C₁-C₈) amide (e.g., formylamino, acetylamino, propylamino, and the like), a C₁-C₁₀ (e.g., C₁-C₈) carboxylic acid (e.g., formic acid, acetic acid, propionic acid, butryic acid, isobutyric acid, pentanoic acid, isopentanoic acids (e.g., isovaleric acid), hexanoic acid, isohexanoic acids, heptanoic acid, isoheptanoic acids, octanoic acid, isooctanoic acids, and the like), or a substituted alkyl group (e.g., an alkyl group containing an additional substituent, such as cyano, nitro, chloro, bromo, iodo, and the like).

In certain embodiments, the 2-methoxy antimycin derivative according to Formula (II) comprises at least one of the following R groups:

R₁ is a C₁-C₁₀ linear alkane (e.g., methyl, ethyl, butyl, pentyl, and the like);

R₂ is either of the following Formula (III) or Formula (IV); 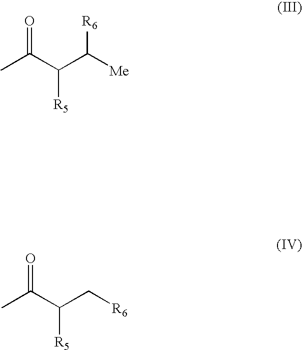
where R₅ and R₆ are each independently selected from the group consisting of a methyl group and a hydrogen;

R₃ is hydrogen, a C₁-C₈ linear or branched alkane (e.g., methyl, ethyl, butyl, isobutyl, pentyl, isopentyl, and the like), hydroxyl, a C₁-C₈ hydroxyalkane (e.g., hydroxymethyl, hydroxyethyl, hydroxypropyl, and the like), a C₁-C₈ amide (e.g., N-formylamino, N-acetylamino, and the like), a C₁-C₈ carboxylic acid (e.g., formic acid, acetic acid, propionic acid, butanoic acid, isobutanoic acids, pentanoic acid, isopentanoic acids (e.g., isovaleric acid), hexanoic acid, isohexanoic acids, heptanoic acid, isoheptanoic acids, octanoic acid, isooctanoic acids, and the like), or a substituted alkyl group (e.g., an alkyl group containing an additional substituent, such as cyano, nitro, chloro, bromo, iodo, and the like); and

R₄is methyl,

with the proviso that the antimycin derivative is not 2-methoxy antimycin A₃. In certain embodiments, the 2-methoxy antimycin derivative according to Formula (II) comprises each of R₁-R₄ as set forth above.

In a preferred embodiment, the 2-methoxy antimycin derivative is of the following Formula (V): 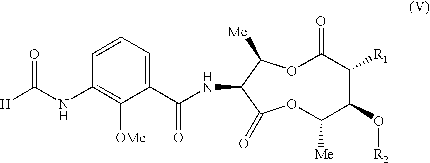
where each of positions R₁ and R₂ can be independently modified, R₂ is an acyl group limited to C₁ -C₈, and with the proviso that the antimycin derivative is not 2-methoxy antimycin A₃. For example, each of R₁ can be hydrogen, a C₁-C₁₀ (e.g., C₁-C₈) linear or branched alkane (e.g., methyl, ethyl, butyl, isobutyl, pentyl, isopentyl, and the like), hydroxyl, a C₁-C₁₀ (e.g., C₁-C₈) hydroxyalkane (e.g., hydroxymethyl, hydroxyethyl, hydroxypropyl, and the like), amino, an amino halogen salt (e.g., amino chloride, amino bromide, amino iodide, or amino fluoride), a C₁-C₁₀ (e.g., C₁-C₈) di- or tri-alkylamine (e.g., methyl amine, dimethyl amine, ethyl amine, diethyl amine, and the like), a C₁-C₁₀ (e.g., C₁-C₈) amide (e.g., formylamino, acetylamino, propylamino, and the like), a C₁-C₁₀ (e.g., C₁-C₈) carboxylic acid (e.g., formic acid, acetic acid, propionic acid, butryic acid, isobutyric acid, pentanoic acid, isopentanoic acids (e.g., isovaleric acid), hexanoic acid, isohexanoic acids, heptanoic acid, isoheptanoic acids, octanoic acid, isooctanoic acids, and the like), or a substituted alkyl group (e.g., an alkyl group containing an additional substituent, such as cyano, nitro, chloro, bromo, iodo, and the like).

In particular embodiments, the 2-methoxy antimycin derivative according to Formula (V) comprises one or both of the following R groups:

R₁ is a C₁-C₁₀ linear alkane (e.g., methyl, ethyl, butyl, pentyl, and the like); and

R₂ has the structural formula represented by either Formula (III) or Formula (IV) (as set forth supra).
For example, in certain embodiments, the 2-methoxy antimycin derivative according to Formula (V) has the chemical structure represented by any of the following Formulas (VI), (VII), (VIII), or (IX):

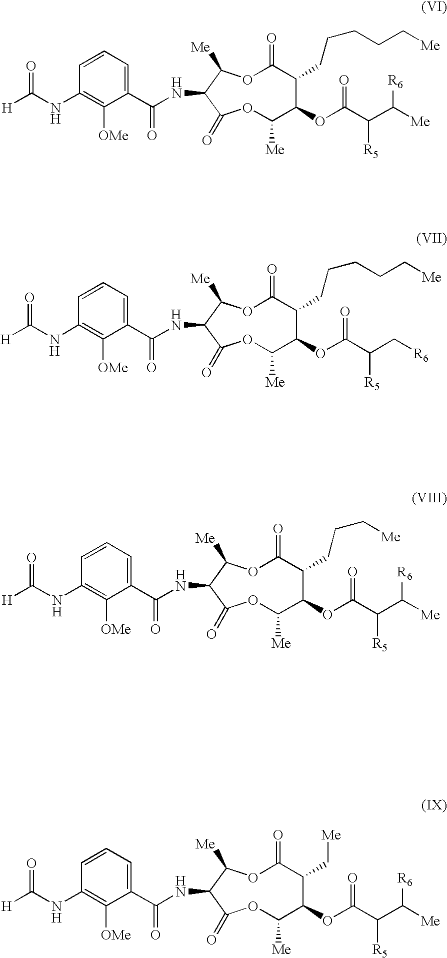

Further, where the 2-methoxy antimycin derivative according to Formula (V) comprises a C₁-C₈ linear alkane at the R₁ position, the linear alkane is preferably an ethyl group, more preferably a butyl group, and even more preferably a hexyl group.

In certain embodiments, the 2-methoxy antimycin derivative is a lactam analogue, in which one or both lactone oxygens are replaced with, for example, nitrogen. For example, a dilactam ring can be substituted for the dilactone ring in an antimycin derivative as set forth above. In one variation, the antimycin derivative having a dilactam ring where both oxygens are replaced and the compound has the following Formula (X) 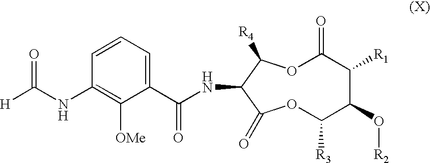
where each of R₁-R₄ can be independently modified as set forth above with respect to Formulas (II), (V), (VI), (VII), (VIII), or (IX). Further, the ester oxygen of the lactone ring of any of the structures described herein can be replaced with nitrogen to provide additional stability to the molecule.

2-methoxy antimycin derivatives can be prepared by chemically modifying an antimycin according to standard methods. Such preparation of the 2-methoxy antimycin derivative includes methylation of the phenolic hydroxyl group of the antimycin. Methylation of the phenolic hydroxyl group can be achieved, for example, by dissolving an antimycin in ethyl ether and passing a stream of diazomethane through the reaction mixture until the yellow color persists. The reaction mixture is then treated with acetic acid until it becomes colorless. The mixture is reduced to dryness under reduced pressure and chromatographed, for example, on silica gel to yield 2-methoxy antimycin. The resulting product can be characterized by NMR, infrared spectroscopy and mass spectroscopy. Additional modifications of the resulting 2-methoxy antimycin compound at any of groups R₁ through R₄, as described supra, can optionally be performed by known methods.

Alternatively, 2-methoxy antimycin derivatives can be prepared by de novo (“total”) chemical synthesis. In particular, compounds where R₃ or R₄ are other than methyl, can not be synthesized from antimycin itself and must therefore be synthesized from other starting materials. For example, Shimano et al. (Tetrahedron 54:12745-74, 1998) have described a total synthetic method for the related antifungal dilactones UK-2A and UK-3A. This total synthesis can be used to prepare 2-methoxy antimycin derivatives. According to this method, 2-methoxy antimycin derivatives having, for example, the structure represented by Formula (V), supra, (e.g., 2-methoxy antimycin A₁ or A₂ and the like) can be modeled as comprising three structural units: N-formyl-2-methoxy-3-amino-benzoic acid, L-threonine, and the dilactone moiety. (See formulae (XI) through (XIII), respectively, where R₁ and R₂ are as described for formulae (II) and (V), supra.) 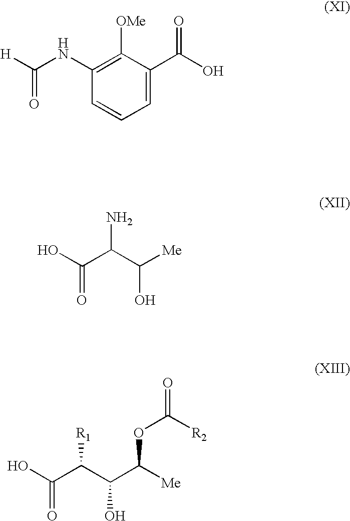
The 2-methoxy antimycin derivative according to Formula (V) can be synthesized by joining these structural units. For example, 2-methoxy antimycin A₁ can be synthesized by joining the structural units of formulae (XI), (XII), and (XIII), where R₁ is hexyl (n-C₆H₁₃) and R₂ has the structure of Formula (III), supra. Similarly, 2-methoxy antimycin A₂ can be synthesized as above using a structural unit of Formula (XII) in which R₁ is hexyl and R₂ has the structure of Formula (IV), supra. Derivatives of one or more of these structural units can also be chemically linked to form other 2-methoxy antimycin derivatives. For example, derivatives of L-threonine include, e.g., L-serine, 2-amino-3-hydroxy-propionic acid, 2-amino-3-hydroxy-hexanoic acid, and the like. Derivatives of the dihydroxypentanoic acid can be prepared by chemical synthesis, as more fully described in the Examples.

Libraries of antimycin derivatives can also be prepared by rational design. (See generally, Cho et al., Pac. Symp. Biocompat. 305-316, 1998; Sun et al., J. Comput. Aided Mol. Des. 12:597-604, 1998, incorporated herein by reference). For example, libraries of 2-methoxy antimycin derivatives can be prepared by syntheses of combinatorial chemical libraries (see generally DeWitt et al., Proc. Nat. Acad. Sci. USA 90:6909-6913, 1993; International Patent Publication WO 94/08051; Baum, Chem. Eng. News, 72:20-25, 1994; Burbaum et al., Proc. Nat. Acad. Sci. USA 92:6027-6031, 1995; Baldwin et al., J. Am. Chem. Soc. 117:5588-5589, 1995; Nestler et al., J. Org. Chem. 59:4723-4724, 1994; Borehardt et al., J. Am. Chem. Soc. 116:373-374, 1994; Ohlmeyer et al., Proc. Nat. Acad. Sci. USA 90:10922-10926, 1993; and Longman, Windhover's In Vivo The Business & Medicine Report 12:23-31, 1994, each incorporated herein by reference.)

The following articles describe methods for selecting starting molecules and/or criteria used in their selection: Martin et al., J. Med. Chem. 38:1431-1436, 1995; Domine et al., J. Med. Chem., 37:973-980, 1994; Abraham et al., J. Pharm. Sci. 83:1085-1100,1994, each incorporated herein by reference.

A “combinatorial library” is a collection of compounds in which the compounds comprising the collection are composed of one or more types of subunits. The subunits can be selected from natural or unnatural moieties, including dienes, benzene compounds, cycloalkanes, lactones, dilactones, amino acids, alkanes, and the like. The compounds of the combinatorial library differ in one or more ways with respect to the number, order, type or types of modifications made to one or more of the subunits comprising the compounds. Alternatively, a combinatorial library may refer to a collection of “core molecules” which vary as to the number, type or position of R groups they contain and/or the identity of molecules composing the core molecule. The collection of compounds is generated in a systematic way. Any method of systematically generating a collection of compounds differing from each other in one or more of the ways set forth above is a combinatorial library.

A combinatorial library can be synthesized on a solid support from one or more solid phase-bound resin starting materials. The library can contain five (5) or more, preferably ten (10) or more, organic molecules which are different from each other (i.e., five (5) different molecules and not five (5) copies of the same molecule). Each of the different molecules (different basic structure and/or different substituents) will be present in an amount such that its presence can be determined by some means (e.g., can be isolated, analyzed, detected with a binding partner or suitable probe). The actual amounts of each different molecule needed so that its presence can be determined can vary due to the actual procedures used and can change as the technologies for isolation, detection and analysis advance. When the molecules are present in substantially equal molar amounts, an amount of about 100 picomoles or more can be detected. Preferred libraries comprise substantially equal molar amounts of each desired reaction product and do not include relatively large or small amounts of any given molecules so that the presence of such molecules dominates or is completely suppressed in any assay.

Combinatorial libraries are generally prepared by derivatizing a starting compound onto a solid-phase support (such as a bead). In general, the solid support has a commercially available resin attached, such as a Rink or Merrifield Resin, and the like. After attachment of the starting compound, substituents are attached to the starting compound. For example, the starting compound can comprise the dilactone moiety, or a precursor thereof. Substituents are added to the starting compound, and can be varied by providing a mixture of reactants comprising the substituents. Examples of suitable substituents include, but are not limited to, the following:

(1) hydrocarbon substituents, that is, aliphatic (e.g., alkyl or alkenyl), alicyclic (e.g., cycloalkyl, cycloalkenyl) substituents, aromatic, aliphatic and alicyclic-substituted aromatic nuclei, and the like, as well as cyclic substituents;

(2) substituted hydrocarbon substituents, that is, those substituents containing nonhydrocarbon radicals which do not alter the predominantly hydrocarbon substituent; those skilled in the art will be aware of such radicals (e.g., halo (especially chloro and fluoro), alkoxy, mercapto, alkylmercapto, nitro, nitroso, sulfoxy, and the like);

(3) hetero substituents, that is, substituents which will, while having predominantly hydrocarbyl character, contain other than carbon atoms. Suitable heteroatoms will be apparent to those of ordinary skill in the art and include, for example, sulfur, oxygen, nitrogen, and such substituents as pyridyl, furanyl, thiophenyl, imidazolyl, and the like. Heteroatoms, and typically no more than one, will be present for each carbon atom in the hydrocarbon-based substituents. Alternatively, there may be no such radicals or heteroatoms in the hydrocarbon-based substituent and it will, therefore, by purely hydrocarbon.

In one embodiment, a combinatorial library of 2-methoxy antimycin derivatives is prepared. For example, the starting compound can be a precursor of the dilactone moiety. A combinatorial library of the dilactone is synthesized using the Shimano synthesis (infra) while varying the substituents added at each step of the synthesis. Following lactonization, the threonine and N-formyl-2-methoxy-3-amino-benzoic acid moieties, or derivatives thereof, are added to the library.

Methods of making combinatorial libraries are known in the art, and include for example, the following: U.S. Pat. Nos. 5,958,792; 5,807,683; 6,004,617; 6,077,954.

Methods of Identifying Biologically Active 2-Methoxy Antimycin Derivatives

Methods are also provided to identify 2-methoxy antimycin (2-OMeA) derivatives that modulate apoptosis and are substantially non-toxic to cells that do not over-express a Bcl-2 family member. In one embodiment, the method generally comprises the steps of contacting a candidate 2-OMeA derivative with a cell that over-expresses a Bcl-2 family member protein; contacting the candidate 2-OMeA derivative with another cell that does not over-express the Bcl-2 family member protein; and determining whether the candidate 2-OMeA derivative modulates the activity of the Bcl-2 family member protein to produce a physiological change in the cell that over-expresses the Bcl-2 family member protein, but does not produce a substantial physiological change in the cell which does not over-express that protein. Physiological changes that are indicative of binding of the candidate 2-OMeA derivative to the Bcl-2 family member protein (e.g., in the hydrophobic pocket) include an affect on cell death, cell shrinkage, chromosome condensation and migration, mitochondria swelling, and/or disruption of mitochondrial transmembrane potential (i.e., the mitochondrial proton gradient), and/or cell death (e.g., as measured by trypan dye exclusion).

In one example, a candidate 2-OMeA derivative is added to mammalian tissue culture cells over-expressing a Bcl-2 family member protein, and separately to cells having normal levels of the Bcl-2 family member protein. Control cells to which no 2-OMeA derivative is added are also included. Methods of expressing Bcl-2 family member proteins in tissue culture cells are well known in the art. (See, e.g., Example 1, U.S. Pat. No. 5,998,583). At various time points after contracting the candidate 2-OMeA derivative (e.g., at about 6 and about 24 hours), the cells from each group are trypsinized, and cell viability is determined by, for example, trypan blue dye exclusion. The number of viable cells are counted and normalized to the control group (i.e., % control=number of viable cells (treated group)/number of viable cells (control group)×100). The candidate 2-OMeA derivative that is effective as an anti-apoptotic agent preferentially induces apoptosis in cells over-expressing the Bcl-2 family member protein, but not in cells having normal levels of the Bcl-2 family member protein.

In another example, the candidate 2-OMeA derivative is added to mammalian tissue culture cells over-expressing a Bcl-2 family member protein and separately to cells having normal levels of the Bcl-2 family member protein. Control cells to which no 2-OMeA derivative are also included. At various time points after administration of the candidate 2-OMeA derivative (e.g., at about 6 and about 24 hours), nuclear morphology is determined by nuclear chromosome staining (e.g., 4′,6-diamidine-2′-phenylindole (DAPI) staining, and the like). Cells in which apoptosis has occurred will exhibit characteristic changes in nuclear morphology, such as chromosome condensation and migration. Methods to monitor other physiological changes typical of apoptosis are disclosed in the Examples (infra).

In another embodiment, reagents and assay conditions which are useful for interrogating 2-OMeA derivatives for utility in the present invention comprise: (1) cells which over-express an anti-apoptotic Bcl-2 family member (e.g., Bcl-2, BCl-x_{L}, Bcl-w, A1, Mcl-1, and the like), (2) aqueous components which produce binding conditions, e.g., physiological buffers, (3) a reporter system, e.g., a cell, or a reporter molecule, and (4) a candidate 2-OMeA derivative being tested. The candidate 2-OMeA derivative can also be screened for toxicity to cells that do not over-express the anti-apoptotic Bcl-2 family member protein.

Typically, 2-OMeA derivatives are initially screened for modulation of activity of cells that over-express the Bcl-2 family member protein. In one particular embodiment, a candidate 2-OMeA derivative is identified by its ability to preferentially induce apoptosis in cells transformed with a gene which encodes at least the BCl-x_{L} BH3 binding domain. The candidate 2-OMeA derivative is then further tested for the absence of, or reduced induction of, apoptosis in a control cell which does not over-express the anti-apoptotic Bcl-2 family member protein (e.g., one that has not been so transformed, or that is transformed with a control vector, or an anti-sense vector). A successful candidate 2-OMeA derivative is one which induces apoptosis preferentially in the cell which over-expresses BCl-x_{L}. In a particular embodiment, candidate 2-OMeA derivatives are assayed for their ability to preferentially induce apoptosis in a murine tumorigenic liver cell line which over-expresses the BCl-x_{L} protein.

In another embodiment, the ability of a candidate 2-OMeA derivative to modulate pore forming activity by a Bcl-2 family member protein is determined. This assay comprises a membrane enclosed vesicle, the vesicle having on its surface a Bcl-2 family member protein, such as BCl-x_{L} or Bcl-2. A reporter present within the vesicle acts as an indicator of pore formation modulation by the candidate 2-OMeA derivative. Suitable reporters include fluorescers, chemiluminescers, radiolabels, enzymes, enzyme cofactors, and the like.

One specific example of this assay comprises preparing large unilamellar vesicles (LUV's) containing a fluorescent reporter molecule. In a particular embodiment, LUV's (e.g., comprising 60% dioleophosphatidylcholine and 40% dioleoylphosphatidyl-glycerol) contain the fluorescent reporter calcein. When an anti-apoptotic Bcl-2 family member protein, is inserted into the vesicle, the fluorescent reporter leaks out of the vesicle. Binding of a candidate 2-OMeA derivative being tested to the anti-apoptotic Bcl-2 family member protein disrupts pore formation, and leakage of the reporter from the vesicle is inhibited or blocked.

In another assay system, the ability of a candidate 2-OMeA derivative to compete with BH3-peptide for binding to the BH3-binding domain of an anti-apoptotic Bcl-2 family member protein is exploited to identify BH3-binding domain ligands. In one example, the ability of a candidate 2-OMeA derivative to compete with the BH3 peptide for binding to the hydrophobic pocket of a Bcl-2 family membrane protein is measured by displacement of labeled BH3 peptide. Suitable labels include fluorescers, chemiluminescers, radiolabels, enzymes, enzyme cofactors, and the like. After addition of the candidate 2-OMeA derivative under suitable binding conditions, the amount of labeled BH3 peptide remaining bound to the Bcl-2 family member protein is determined. Such an assay is useful both for identifying 2-OMeA derivatives that inhibit the biological activity of the Bcl-2 family member protein and to identify 2-OMeA derivatives that block binding of pro-apoptotic Bcl-2 family member proteins to the anti-apoptotic protein without affecting the biological activity.

In other embodiments, combinatorial libraries of candidate 2-OMeA derivatives can be screened for biological activity using any of the methods described herein. For example, combinatorial library 2-OMeA derivatives that modulate apoptosis, or that bind to Bcl-2 family member proteins, can be identified. One such method for testing a candidate 2-OMeA derivative for the ability to bind to and potentially modulate apoptosis is as follows: exposing at least one candidate 2-OMeA derivative from the combinatorial library to a Bcl-2 family member protein for a time sufficient to allow binding of the combinatorial library 2-OMeA derivative to the protein; removing non-bound 2-OMeA derivative; and determining the presence of the candidate 2-OMeA derivative bound to the protein. Also or interest is the use of a combination of methods to screen of derivatives of interest. Of particular interest is a combination of methods that include selecting deratives that 1) demonstrate the ability to selectively induce apoptosis in a cell line that over-expresses the Bcl-2 family member anti-apoptosis protein as compared to a wild-type cell; 2) demonstrate the ability to inhibit pore formation in a lipid-enclosed vesicle that has on it's surface the Bcl-2 family member anti-apoptotic protein; 3) demonstrate the ability to be competitively displaced from binding to the Bcl-2 family member anti-apoptotic protein by a BH3-peptide; and 4) lack the ability to competitively displace the BH3-peptide from binding to the Bcl-2 family member anti-apoptotic protein. Agents that demonstrate each of these activities can be distinguished from agents that bind to the hydrophobic pocket of a Bcl-2 family anti-apoptotic protein but fail to modulate apoptosis.

Another method utilizing this approach that can be pursued in the identification of such candidate 2-OMeA derivatives includes the attachment of a combinatorial library, or a portion thereof, to a solid matrix, such as agarose or plastic beads, microtiter wells, petri dishes, or membranes composed of, for example, nylon or nitrocellulose, and the subsequent incubation of the attached combinatorial library molecule in the presence of a Bcl-2 family member protein. Attachment to the solid support can be direct or by means of a combinatorial-library-2-OMeA derivative-specific antibody bound directly to the solid support. After incubation, unbound 2-OMeA derivatives are washed away, and protein-bound 2-OMeA derivatives are recovered. By utilizing this procedure, large numbers of candidate 2-OMeA derivatives can be simultaneously screened for Bcl-2 family member protein-binding activity.

Particularly useful 2-OMeA derivatives are those that i) induce apoptosis selectively in a cell line that over-expresses a Bcl-2 family member anti-apoptotic protein, ii) inhibit Bcl-2 family member anti-apoptotic protein mediated pore formation as measured by leakge of a reporter from a membrane vesicle, and iii) binding to the hydrophobic pocket of a Bcl-2 family member as indicated by competitive displacement by a BH3-peptide and a lack of competitive displacement by the 2-OMeA deriviatve of bound BH3-peptide. Am example of an assay to measure each of the above characteristics is described above.

Identification of Biologically Active 2-OMeA Derivatives Using Bcl-2 Family Member Mutant Proteins

Biologically active 2-OMeA derivatives can also be identified using Bcl-2 family member protein mutants. 2-OMeA derivatives having desired activity for modulating anti-apoptotic Bcl-2 family member proteins exhibit reduced binding affinity and activity for Bcl-2 family member proteins having one or more specific amino acid substitutions in the hydrophobic groove formed by the BH1, BH2, and BH3 domains of the protein. The Bcl-2 family member mutant proteins include those having at least one mutation in the hydrophobic groove that corresponds to one of the following BCl-x_{L} mutations: substitution of Glutamic acid at position 92 with Leucine (E92L), substitution of Phenylalanine at position 97 with Tryptophan (F97W), substitution of Leucine at position 130 with Alanine, (L130A), substitution of Alanine at position 142 with Leucine (A142L), substitution of Phenylalanine at position 146 with Leucine (F146L), or the substitution of Tyrosine at position 195 with Glycine (Y195G). For example, in screening for 2-OMeA derivatives or other compositions that modulate apoptosis by binding to BCl-x_{L} at the same or a similar site, BCl-x_{L} mutants having a E92L, F97W, L130A, A142L, F146L, or Y195G substitution can be used. The A142L and F146L mutants appear to be the most informative for in vivo activity or binding. Further, the Bcl-2 family member protein mutants can be used to screen for agents related to 2-OMeA derivatives that share the same, or have a similar, binding sites within the hydrophobic pocket of the Bcl-2 family member proteins.

The Bcl-2 family member protein mutants described above exhibit no substantial alteration of tertiary structure relative to the corresponding wild-type Bcl-2 family member protein. Further, the Bcl-2 family member protein mutants have (1) decreased binding affinity for biologically active 2-methoxy antimycin derivatives (e.g., 2-OMeA₁, 2-OMeA₂, 2-OMeA₃, or 2-OMeA₅); (2) reduced sensitivity to biologically active 2-methoxy antimycin derivatives (e.g., 2-OMeA₁, 2-OMeA₂, 2-OMeA₃, or 2-OMeA₅) when expressed in cells assayed for physiologic changes associated with apoptosis; and/or (3) reduced inhibition of in vitro pore-formation activity in response to a biologically active 2-methoxy antimycin derivative. (See, e.g., Table 2, which summarizes the characterization of suitable BCl-x_{L} mutants, E92L, F97W, L130A, A142L, F146L, and Y195G; and Table 3, which summarizes Cα trace overlays of Bcl-x_{L} point mutant structures with wild-type BCl-x_{L}; (See also, e.g., antimycin binding assays, cell-based assays for apoptosis-associated changes, and pore-formation assays described herein.)

TABLE 2
Summary of Characterization of Bcl-x_{L} Mutants

| | | In vivo | In vivo | In vitro | In vitro | Major |
|---|---|---|---|---|---|---|
| | Predicted Effect on | STS | Antimycin | Antimycin | Pore | Structural |
| Mutation | Antimycin Interaction | Sensitivity | Sensitivity | Binding | Inhibition | Effects |
| WT | Binds in | − | + | + | + | *** |
| | BH3 pocket | | | | | |
| E92L | No | − | + | + | + | No |
| F97W | Clashing | − | +/− | − | − | No |
| | Contact | | | | | |
| L130A | Loss of | − | − | nd | nd | nd |
| | Contact | | | | | |
| A142L | Loss of | − | − | − | − | nd |
| | Contact | | | | | |
| F146L | Loss of | − | − | +/− | + | No |
| | Contact | | | | | |
| F146W | Clashing | + | +/− | +/− | + | Yes |
| | Contact | | | | | |
| Y195G | Loss of H- | − | − | nd | nd | nd |
| | bonding | | | | | |
| A200L | None | − | + | nd | nd | No |

TABLE 3
Summary of Cαtrace overlays of Bcl-x_{L} point
mutants with wild-type Bcl-x_{L}.

| | Resolution | R, R_{free} (%) | RMSD (Cα) | Major Effect |
|---|---|---|---|---|
| WT | 1.9 Å | 23.8, 24.6 | — | — |
| E92L | 2.1 Å | 23.8, 25.6 | 0.21 Å | None |
| F97W | 2.7 Å | 19.7, 24.0 | 0.34 Å | None |
| F146L | 2.1 Å | 25.1, 27.2 | 0.25 Å | None |
| F146W | 2.2 Å | 25.4, 28.6 | 1.23/3.67 | Shift of α3 |
| A200L | 2.2 Å | 25.3, 27.6 | 0.21 Å | None |

These mutant Bcl-2 family member proteins can be used in any of the assays described above to determine whether the candidate 2-OMeA derivative exhibits reduced binding activity or biological activity for the mutant protein relative to the corresponding Bcl-2 family member protein not having the mutation (e.g., the wild-type Bcl-2 family member protein). For example, the candidate 2-OMeA derivative can be contacted separately with each of two cell populations, the first cell populations over-expressing wild-type BCl-x_{L} and the second cell population that over-expresses a corresponding BCl-x_{L} protein having the E92L, F97W, L130A, A142L, F146L, and/or Y195G mutation. The cell populations are then assayed for an apoptosis-associated physiological effect to determine whether the candidate 2-OMeA derivative produces a reduced effect in those cells over-expressing the mutant Bcl-x_{L} protein relative to those cells over-expressing the wild-type BCl-x_{L} protein. Those candidate 2-OMeA derivatives having a reduced effect on those cells expressing the mutant BCl-x_{L} protein are apoptosis-modulating 2-methoxy antimycin derivatives that bind to the hydrophobic groove of BCl-x_{L}.

In another example, computer-based methods can be used to identify biologically active 2-OMeA derivatives by using a “molecular docking” algorithm to score candidate 2-OMeA derivatives for binding to each of a Bcl-2 family member protein and a corresponding mutant protein as described supra. Those candidate compounds that demonstrate a lower score for binding to the mutant protein relative to the corresponding Bcl-2 family member protein (e.g., a mutant Bcl-x_{L} protein (having a E92L, F97W, L130A, A142L, F146L, or Y195G mutation) and the wild-type Bcl-x_{L} protein, respectively) can be further evaluated in biological assays as described supra to verify biological activity.

Computer-based techniques for examining potential ligands (e.g., candidate compounds) for binding to target molecules are well-known in the art. (See, e.g., Kuntz et al., J. Mol. Biol. 161:269-288, 1982; Kuntz, Science 257:1078-1082, 1992; Ewing and Kuntz, J. Comput. Chem. 18:1175-1189, 1997). For example, the DOCK suite of programs is designed to find possible orientations of a ligand in a receptor site. (See, e.g., Ewing and Kuntz, supra.) The orientation of the ligand is evaluated with a shape scoring function (an empirical function resembling the van der Waals attractive energy) and/or a function approximating the ligand-receptor binding energy (which is taken to be approximately the sum of the van der Waals and electrostatic interaction energies). After an initial orientation and scoring evaluation, a grid-based rigid body minimization is carried out for the ligand to locate the nearest local energy minimum within the receptor binding site. The position and conformation of each docked molecule can be optimized, for example, using the single anchor search and torsion minimization method of DOCK4.0. (See, e.g., Ewing and Kuntz, supra; Kuntz, supra.)

In addition, heuristic docking and consensus scoring strategies can be used in the computer-based identification of biologically active 2-OMeA derivatives (i.e., different docking and scoring methods can be applied to evaluate the screening results). For example, following a primary screening using, e.g., DOCK4.0 (supra), top-scoring compounds can be re-scored using other docking algorithms such as, for example, GOLD, FlexX, PM (see Muegge and Martin, J. Med. Chem. 42:791, 1999, and/or AutoDock3.0 (see Morris et al., J. Comput. Chem. 19:1639-1662, 1998). Optionally, following a primary and any subsequent screen(s) using individual docking algorithms, a consensus score (Cscore) can be determined by combining results from any of the individual docking programs used to score the candidate compounds (see Clark et al., J. Mol. Graph Model 20:281-295, 2002). Based on the scoring results from a secondary or other subsequent screen, a subset, for example, of the top-scoring molecules from the primary screen can be selected for further analyses (e.g., a tertiary virtual screen or, alternatively or additionally, biological screening assays such as, for example, any of the assays described herein or otherwise known in the art).

Identification of Biologically Active 2-OMeA Derivatives Using Glucose Uptake or Lactate Production Assays

Biologically active 2-OMeA derivatives can also be identified by evaluating the ability of the agents to modulate glucose uptake and/or lactate production in cells expressing an anti-apoptotic Bcl-2 family member protein. Apoptosis-modulating 2-methoxy antimycin derivatives increase cellular glucose uptake or lactate production in proportion to the level of expression of a Bcl-2 family member target protein. Therefore, a biologically active 2-OMeA derivative can be identified by contacting a candidate 2-OMeA derivative independently to each of two cell populations expressing a Bcl-2 family member protein, where one cell population has a higher level of expression of the Bcl-2 family member protein relative to the other cell; determining the level of glucose uptake or lactate production in each cell; and determining whether the cell having higher expression of the Bcl-2 family member protein has a higher level of glucose uptake or lactate production relative to the cell having lower expression of the Bcl-2 family member protein.

Methods for assaying glucose production or lactate production are well-known in the art. (See, e.g., Schultz and Ruzicka, Analyst 127:1293-1298, 2002; Schultz et al., Analyst 127:1583-1588, 2002). For example, glucose and lactate concentrations can be assayed as substrates in first-order NAD-linked enzymatic reactions, with NADH generation monitored by absorbance at 340 nm. The glucose reagent can include final concentrations of, e.g., >1500 U hexokinase, >3200 U glucose-6-phosphate dehydrogenase, 2.1 mM ATP, and 2.5 mM NAD⁺. The lactate reagent can include final concentrations of, e.g., 2000 U/ml LDH and 2.5 mM NAD⁺ in glycine buffer. For experiments, cells are typically maintained in an appropriate buffer (e.g., Hanks balanced salt solution (HBSS)). The candidate compound is contacted with the cells, followed by an incubation period to allow depletion of glucose and accumulation of lactate. A fixed volume of tissue culture supernatant (e.g., buffer solution incubated in the presence of treated cells) is then added to glucose or lactate reagent and 340 nm absorbance is recorded. Further, the determination of glucose uptake or lactate production can be carried out using automated microsequential injection analysis of cells attached to beads. (See, e.g., Schultz and Ruzicka, supra; Schultz et al., supra; Example 20, infra).

Identification of 2-OMeA derivatives having reduced binding affinity for cytochrome b

In a preferred embodiment, the biologically active 2-OMeA derivative exhibits reduced binding affinity for cytochrome b. Thus, candidate 2-OMeA derivatives can be screened for such reduced binding affinity for cytochrome b to identify desired derivatives. Methods for measuring binding to cytochrome b include measuring the effect of the candidate 2-OMeA derivative on cytochrome bc, activity according to the methodology described by Miyoshi et al. (Biochim. Biophys. Acta 1229:149-154, 1995). Briefly, submitochondrial particles are prepared from bovine heart mitochondria according to standard methods. (See, e.g., Matsuno-Yagi and Hatefi, J. Biol. Chem. 260:14424-14427, 1985). The particles are treated with sodium deoxycholate (0.3 mg/mg protein) before dilution with reaction buffer. (See, e.g., Esposti and Lenaz, Biochim. Biophys. Acta 682:189-200, 1982). Cytochrome bc₁ complex activity is measured at 30° C. as the rate of cytochrome c reduction with an electron donor, such as 2,3-dimethoxy-5-methyl-6-n-decyl-1,4-benzoquinol (DBH). The reaction buffer can comprise 0.25 M sucrose, 1 mM MgCl₂, 2 mM KCN, 20 μM cytochrome c and 50 mM phosphate buffer (pH 7.4). The final mitochondrial protein concentration is 15 μg/ml.

In another embodiment, ATP production by mitochondria is measured as an indication of cytochrome b activity. Briefly, the candidate 2-OMeA derivative is added to the cells. After a 1 hour incubation, cells are harvested, and intracellular ATP concentrations are determined, for example, by an ATP-dependent luciferase-luciferin assay (Sigma, St. Louis, Mo.). An antimycin, such as the mixture of antimycins provided as antimycin A, and/or A₃, can be used as a control. Reduced cytochrome B binding is indicated by a smaller reduction in intracellular ATP levels by the candidate 2-OMeA derivative than by the antimycin control.

Methods of Using the 2-Methoxy Antimycin Derivatives

2-methoxy antimycin derivatives of the present invention are useful for treating cells in which the cell death signal is down-regulated and the affected cell has an inappropriately diminished propensity for cell death, which is referred to herein as being in a “decreased apoptotic state.” The invention further provides methods for the administration to a subject of a therapeutically effective amount of a 2-OMeA derivative to treat an apoptosis-associated disease in which it is desirable to induce apoptosis in certain types of cells, such as virus-infected or autoantibody-expressing cells.

In a specific embodiment, a method of treating a cancer characterized by the over-expression of a Bcl-2 family member is provided. Examples of cancers known to be associated with over-expression of a Bcl-2 family memer and which can be treated according to the methods provided herein are shown in Table 4.

In some cases, the treatment of the cancer can include the treatment of solid tumors or the treatment of leukemias. For example, the cancer can be of the skin, breast, brain, cervix, testis, and the like. More particularly, cancers may include, but are not limited to, the following organs or systems: cardiac, lung, gastrointestinal, genitourinary tract, liver, bone, nervous system, gynecological, hematologic, skin, and adrenal glands. More particularly, the methods herein can be used for treating gliomas (Schwannoma, glioblastoma, astrocytoma), neuroblastoma, pheochromocytoma, paraganlioma, meningioma, adrenal cortical carcinoma, kidney cancer, vascular cancer of various types, osteoblastic osteocarcoma, prostate cancer, ovarian cancer, uterine leiomyomas, salivary gland cancer, choroid plexus carcinoma, mammary cancer, pancreatic cancer, colon cancer, B and T cell lymphomas, acute and chronic myeloid or lymphoid leukemias, and multiple myeloma. Further, treatment may include pre-malignant conditions associated with any of the above cancers (e.g., colon adenomas, myelodysplastic syndrome).

Typically, the 2-OMeA derivative is substantially purified prior to administration. The subject can be an animal, including, but not limited to, cows, pigs, horses, chickens, cats, dogs, and the like, and is typically a mammal, and in a particular embodiment human. In another specific embodiment, a non-human mammal is the subject.

The 2-OMeA derivative generally comprises an antimycin derivative of Formula II as set forth supra. In certain embodiments, the 2-OMeA derivative is of Formula V, such as, for example, a 2-OMeA derivative of Formula VI, VII, VIII, or IX.

Various delivery systems are known and can be used to administer a 2-OMeA derivative, such as, for example, encapsulation in liposomes, microparticles, microcapsules, recombinant cells capable of producing the derivative, receptor-mediated endocytosis (see, e.g., Wu and Wu, J. Biol. Chem. 262:4429-4432, 1987), and the like. The 2-OMeA derivatives are administered as therapeutic or pharmaceutical compositions by any suitable route known to the skilled artisan including, for example, intravenous, subcutaneous, intramuscular, intradermal, transdermal, intrathecal, intracerebral, intraperitoneal, intransal, epidural, and oral routes. Administration can be either rapid as by injection or over a period of time as by slow infusion or administration of slow release formulations. For treating tissues in the central nervous system, administration can be by injection or infusion into the cerebrospinal fluid (CSF). When it is intended that a 2-OMeA derivative be administered to cells in the central nervous system, administration can be with one or more other components

TABLE 4
Percentages of common human cancers with elevated
levels of Bcl-2 or Bcl-x_{L} expression

| Tumor | Bcl-2 | Bcl-x_{L} |
|---|---|---|
| Lymphoma | Hodgkin's - 47-65% | Hodgkin's - 48-94% |
| | NHL - 9-57% | NHL - 25-45% |
| Leukemia | AML- 13-20% | AML - 38% |
| | ALL - 89-92% | ALL -13% |
| | CML - 33-54% | |
| | CLL - 70-95% | |
| Myeloma | 43% | 29% |
| Lung | NSCLC-squamous - 25% | Most NSCLC, SCLC |
| | adenoca - 12% | |
| | SCLC - 83-90% | |
| Colorectal | Adenoma - 65-98% | Adenoma - 50% |
| | Carcinoma - 46-60% | Carcinoma - 60% |
| Breast | 80% | 43-75% |
| Pancreas | 23% | 88% |
| Urogenital | Bladder - 24% | Bladder - 80.9% |
| | Renal - 53% | Renal - 38% |
| Liver | Rare | 95+% |
| Ovary | 30-39% | 62% |
| Brain | Medulloblastoma - 5-25% | Medulloblastoma - 56% |
| | Glioma - 28-92% | Glioma - 98% |
| | Oligodendroglioma -16-60% | Oligodendroglioma - <5% |
| Esophagus | SCC - 45% | Adenocarcinoma - 90% |
| | Adenocarcinoma - 20-40% | |

capable of promoting penetration of the derivative across the blood-brain barrier. In addition, it can be desirable to introduce a 2-OMeA derivative into the target tissue by any suitable route, including intravenous and intrathecal injection. Pulmonary administration can also be employed, such as, for example, by use of an inhaler or nebulizer, and formulation of the 2-OMeA derivative with an aerosolizing agent. In certain embodiments, the 2-OMeA derivative is co-administered with an inhibitor of esterase activity to further stabilize the 2-OMeA derivative.

Pharmaceutical compositions can also be administered orally in any orally acceptable dosage form including, but not limited to, capsules, tablets, caplets, lozenges, aqueous suspensions or solutions. In the case of tablets for oral use, carriers which are commonly used include lactose and corn starch. Lubricating aids, such as magnesium stearate, are also typically added. For oral administration in a capsule form, useful diluents include lactose and dried corn starch. When aqueous suspensions are required, the agent can be combined with emulsifying and suspending aids. If desired, certain sweeteners, flavorants, or colorants can also be used.

Further, the 2-OMeA derivative can be combined with any other tumor and/or cancer therapy. The therapy can include, for example and not by way of limitation, surgery, radiation, and chemotherapy either individually or in any combination. Chemotherapy can include any current known or yet to be discovered chemotherapeutic agent including, for example, paclitaxel, doxorubicin, etoposide, melphalan, daunorubicin, 5-fluorouracil, cisplatin, paraplatin, and the like, either individually or in any combination. In addition, as the 2-OMeA derivatives of the present invention have been found to increase the rate of glucose uptake, cytotoxic agents that share the cellular glucose uptake pathway or have an increased toxicity in cells with increased glucose uptake and/or lactate production can be used in combination with these agents. Also, as the present inventors have observed sensitization to 2-OMeA by reducing glucose uptake (e.g., lowering glucose concentrations in the media from about 200 mg/dl to about 100 mg/dl), yet other embodiments of the present invention include the use of a 2-OMeA derivative in combination with a a hypoglycemic agent.

In a specific embodiment, it can be desirable to administer the 2-OMeA derivative locally to the area in need of treatment; this administration can be achieved by, for example, and not by way of limitation, local infusion during surgery, topical application (e.g., in conjunction with a wound dressing after surgery), by injection, by means of a catheter, by means of a suppository, or by means of an implant, the implant being of a porous, non-porous, or gelatinous material, including membranes such as silastic membranes, or fibers. In one embodiment, administration can be by direct injection at the site (or former site) of a malignant tumor or neoplastic or pre-neoplastic tissue.

In another embodiment, the 2-OMeA derivative can be delivered in a vesicle, in particular a liposome (see, e.g., Langer, Science 249:1527-1533, 1990; Treat et al., In Liposomes in the Therapy of Infectious Disease and Cancer, Lopez-Berestein and Fidler (eds.), Liss, N.Y., pp. 353-365, 1989; Lopez-Berestein, supra, pp. 317-327).

In yet another embodiment, the agent can be delivered in a controlled release system. In one embodiment, a pump can be used (see, e.g., Langer, supra; Sefton, CRC Crit. Ref. Biomed. Eng. 14:201, 1987; Buchwald et al., Surgery 88:507, 1980; Saudek et al., N. Engl. J. Med. 321:574, 1989). In another embodiment, polymeric materials can be used (see, e.g., Medical Applications of Controlled Release, Langer and Wise (eds.), CRC Pres., Boca Raton, Fla., 1974; Controlled Drug Bioavailability, Drug Product Design and Performance, Smolen and Ball (eds.), Wiley, N.Y., 1984; Ranger and Peppas, J. Macromol. Sci. Rev. Macromol. Chem. 23:61, 1983; see also Levy et al., Science 228:190, 1985; During et al., Ann. Neurol. 25:351, 1989; Howard et al., J. Neurosurg. 71:105, 1989). In yet another embodiment, a controlled release system can be placed in proximity of the therapeutic target, thus requiring only a fraction of the systemic dose (see, e.g., Goodson, Medical Applications of Controlled Release, supra, Vol. 2, pp. 115-138, 1984). Other controlled release systems are discussed in, for example, the review by Langer (Science 249:1527-1533, 1990).

The present invention also provides pharmaceutical compositions. Such compositions comprise a pharmaceutically acceptable carrier and a therapeutically effective amount of a 2-OMeA derivative. The term “pharmaceutically acceptable” means approved by a regulatory agency of the Federal or a state government or listed in the U.S. Pharmacopeia or other generally recognized pharmacopeia for use in animals, and more typically in humans. The term “carrier” refers to a diluent, adjuvant, excipient, stabilizer, vehicle, or any combination thereof, with which the agent is formulated for administration. Pharmaceutical carriers can be sterile liquids, such as water and oils, including those of petroleum, animal, vegetable or synthetic origin, such as peanut oil, soybean oil, mineral oil, sesame oil, and the like. Water is a typical carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The composition, if desired, can also contain minor amounts of wetting or emulsifying agents, or pH buffering agents. Pharmaceutical compositions can take the form of solutions, suspensions, emulsion, tablets, pills, capsules, powders, sustained-release formulations, and the like. The composition can be formulated as a suppository, with traditional binders and carriers such as triglycerides. In addition, in certain embodiments, the pharmaceutical composition includes an inhibiter of esterase activity as a stabilizing agent.

Oral formulations can include standard carriers such as pharmaceutical grades of mannitol, lactose, starch, magnesium stearate, sodium saccharine, cellulose, magnesium carbonate, and the like. Examples of suitable pharmaceutical carriers are described in, for example, Remington's Pharmaceutical Sciences, by E. W. Martin. Such compositions will contain a therapeutically effective amount of the 2-OMeA derivative, typically in purified form, together with a suitable amount of carrier so as to provide a formulation proper for administration to the subject. The formulation should suit the mode of administration.

In one embodiment, the 2-OMeA derivative is formulated in accordance with routine procedures as a pharmaceutical composition adapted for intravenous administration to human beings. Typically, pharmaceutical compositions for intravenous administration are solutions in sterile isotonic aqueous buffer. Where necessary, the composition can also include a solubilizing agent and a local anesthetic to ease pain at the site of the injection. Generally, the ingredients are supplied either separately or mixed together in unit dosage form. For example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

The 2-OMeA derivatives of the invention can be formulated as neutral or salt forms. Pharmaceutically acceptable salts include those formed with free amino groups such as those derived from hydrochloric, phosphoric, acetic, oxalic, tartaric acids, and the like, and those formed with free carboxyl groups such as those derived from sodium, potassium, ammonium, calcium, ferric hydroxides, isopropylamine, triethylamine, 2-ethylamino ethanol, histidine, procaine, and the like.

The amount of the 2-OMeA derivative that is combined with the carrier to produce a single dosage form will vary, depending upon the nature of that agent and the composition of the dosage form. It should be understood, however, that a specific dosage and treatment regime for any particular patient or disease state will depend upon a variety of factors, including the age, body weight, general health, sex, diet, time of administration, rate of excretion, drug combination, the judgment of the treating physician, and the severity of the particular disease being treated. The amount of active agent will also depend upon the specific activity of the 2-OMeA derivative and whether that agent is co-administered with any other therapeutic or prophylactic ingredients. Determination of therapeutically effective dosages is typically based on animal model studies and is guided by determining effective dosages and administration protocols that significantly reduce the occurrence or severity of the apoptosis-associated disease in model subjects (e.g., in the case of treatment of malignancies, a tumor xenograft model in mice can be used (see, e.g., Example 20). For treatment of human subjects, such animal model studies are typically followed up by human clinical trials. A non-limiting range for a therapeutically effective amount of the 2-OMeA derivative is about 0.001 mg/kg and about 100 mg/kg body weight per day, and in more specific embodiments between about 0.001 mg/kg and about 50 mg/kg, between about 0.01 mg/kg and about 20 mg/kg. between about 0.1 and about 10 mg/kg, or between about 0.1 mg/kg and about 5 mg/kg body weight per day.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

The following examples are provided merely as illustrative of various aspects of the invention and shall not be construed to limit the invention in any way.

### EXAMPLES

### Example 1

To examine the sensitivity of cells over-expressing BCl-x_{L} to various mitochondrial inhibitors and apoptosis inducers, cell lines over-expressing Bcl-x_{L} were prepared and tested.

Briefly, a DNA fragment encoding the full-length mouse BCl-x_{L} cDNA was isolated from the plasmid PBS.BCL-x_{L} (Tzung et al., Am. J. Path. 150:1985-1995, 1997, incorporated herein by reference in its entirety) by digestion with the restriction endonuclease EcoRI. This EcoRI fragment was cloned into the EcoRI site of the mammalian expression vector pSFFV (Fuhlbrigge et al., Proc. Nat. Acad. Sci. USA 85:5649-5653, 1988) in both sense and antisense orientations, to form expression plasmids pSFFV.Bcl-x_{L}(sense) or pSFFV.Bcl-x_{L}(antisense), respectively. The tumorigenic murine hepatocyte cell line TAMH was transfected by lipofection (Lipofectamine, Life Technologies, Rockville, Md., according to the manufacturer's recommendations) with the plasmids pSFFV.neo (the control), pSFFV.Bcl-x_{L}(sense) or pSFFV.Bcl-x_{L}(antisense). Characterization of and culture conditions for the cell lines have been previously published (Wu et al., Proc. Nat. Acad. Sci. USA 91:674-678, 1994;. Wu et al., Cancer Res. 54:5964-5973, 1994, each incorporated herein by reference in its entirety). Transfectants were selected for the acquisition of neomycin resistance by growing cells in the presence of 750 μg/ml of G418. Bulk transfectants were cloned by limiting dilution and individual clones were screened by immunoblot analysis to determine the level of BCl-x_{L} protein expression as described below.

BCl-x_{L} protein expression was determined by Western blot analysis. Cell pellets or purified mitochondrial pellets were lysed in 1% Triton X-100, 5 mM Tris (pH 8.0) and 150 mM NaCl. Each lane was loaded with 20 μg of protein and electrophoresed (120 V) on a 12% SDS-polyacrylamide gel. Proteins were then electrically transferred to a nitrocellulose membrane. Immunodetection was performed using the rabbit anti-Bcl-x_{L} polyclonal antibody 13.6 (Gottschalk et al. Proc. Nat. Acad. Sci. USA 91:7350-7354, 1994, which is incorporated by reference herein in its entirety) followed by a biotinylated goat anti-rabbit antibody (Vector, Burlingame, Calif.; 1:500 dilution) and horseradish peroxidase conjugated streptavidin (Zymed, S. San Francisco, Calif.; 1:1000 dilution). Chemiluminescence (ECL, Amersham, Arlington Heights, Ill.) was used for detection. Expression of BCl-x_{L} expression was indicated by the appearance of a band of approximately 29 kDa.

BCl-x_{L} protein levels were determined by comparing the intensity of the 29 kDa band on a Western (immunoblot) blot between selected transfectants and the parental TAMH hepatocyte cell line. TABX2S cells (transfected with pSFFV.Bcl-x_{L} (sense)) was found to express a 4- to 5-fold higher level of Bcl-x_{L} protein as compared with the parental (control) TAMH.neo cells. The antisense transfectant TABX1A (transfected with PSFFV.Bcl-x_{L} (anti-sense)), on the other hand, was found to express little or no BCl-x_{L} protein.

Mitochondrial expression of BCl-x_{L} protein was examined by Western blot analysis of mitochondrial lysates prepared from TABX2S cells and TABX1A cells. Briefly, mitochondrial pellets were prepared by centrifugation and the pellets were lysed in 1% Triton,5 mM Tris (pH 8.0) and 150 mM NaCl. Each lane of a 12% SDS-polyacrylamide gel was loaded with 20 μg of protein and electrophoresed (120 V) through the gel. Proteins were then electrically transferred to a nitrocellulose membrane. Detection of BCl-x_{L} protein was as described above. Consistent with the results for overall cellular expression of BCl-x_{L} protein, the level of mitochondrial BCl-x_{L} protein was approximately 6 fold higher in TABX2S (pSFFV.Bcl-x_{L} (sense)) cells than TAMH.neo cells (control).

Selected transfectants were then tested for whole cell sensitivity to several apoptotic agents. Transfected cells were cultured to reach approximately 80% confluency prior to plating an equal number of cells from selected clones on 12-well tissue culture plates. The transplanted cells were treated with the following apoptotic agents: 5 μM doxorubicin for 48 hours; 5 μM cisplatin for 48 hours; or with 200 U/ml tumor necrosis factor (TNF) plus 1 μg/ml actinomycin D for 18 hours. Cell viability was determined by trypan blue dye exclusion. The percentage of viable cells was calculated by the number of viable cells (treated with a particular apoptogenic agent) divided by the number in the control group (untreated).

The sensitivity of the tested transfectants to treatment with apoptotic agents was inversely correlated with the level of BCl-x_{L} expression. Cells over-expressing BCl-x_{L} were less sensitive to the apoptogenic agent than control cells. For example, after treatment with doxorubicin (5 μM) for 48 hours, 50% of control TAMH.neo cells (control), 88% of TABX2S cells (over-expressing BCl-x_{L}) and 20% of TABX1A cells (under-expressing Bcl-x_{L}) remained viable. A similar trend was observed with cisplatin or TNF treatment. Thus, cells over-expressing BCl-x_{L} were less sensitive to the apoptogenic agent than control cells, and conversely, cells expressing an anti-sense construct, (pSFFV.Bcl-x_{L} (antisense)) were more sensitive than control cells.

TABX2S cells and TABX1A cells were also examined for the effects of various mitochondrial inhibitors. To test the apoptotic responses of these cells following direct perturbation of mitochondrial function, the cells were treated with rotenone (a mitochondrial complex I inhibitor), sodium azide (a mitochondrial complex IV inhibitor), antimycin A (a mitochondrial complex III inhibitor), valinomycin (an ionophore), and oligomycin (an ATP synthase or mitochondrial complex V inhibitor). Briefly, antimycin A (Sigma, St. Louis, Mo.) and rotenone were dissolved in DMSO to form a stock solution, while valinomycin and oligomycin were dissolved in chloroform and ethanol, respectively, to form stock solutions. Azide was diluted from an aqueous stock solution. Antimycin A (a mixture of antimycins A₁-A₄) (0 to 5 μg/ml), rotenone (0 to 2.5 μg/ml), valinomycin (0 to 10 μg/ml), oligomycin (0 to 10 μM), and azide (0 to 2 μM) were serially diluted into culture medium. Controls received an equivalent concentration of diluent. At various time points after drug treatment, cells were trypsinized, and cell viability was determined by trypan blue dye exclusion. The number of viable cells were counted and normalized to control group (i.e., % control=number of viable cells (treated group)/number of viable cells (control group)×100).

TABX2S cells were found to be markedly more sensitive than TABX1A and TAMH.neo cells to antimycin A over a wide range of concentrations. When the LD₅₀ of antimycin A was estimated from the dose-response curve, a seven-fold difference was found between TABX2S cells (LD₅₀=1.2 μM) and TABX1A or TAMH.neo cells (LD₅₀=8.3 μM). Following the addition of antimycin A to the cell culture, cell death was readily apparent within 2 hours in TABX2S cells, but not in TABX1A cells. The morphology of the dying cells was examined by light microscopy, which indicated that the TABX2S cells treated with antimycin A had an appearance consistent with apoptosis. The cells were also stained with Annexin V-EGFP and propidium iodide, according to the manufacturer's instructions (Clontech, Palo Alto, Calif.). TABX2S cells treated with antimycin A exhibited a redistribution of phosphatidylserine to the outer plasma membrane, which is consistent with the induction of apoptosis. There were no significant differences in the sensitivity of the two cell lines to rotenone, sodium azide, valinomycin or oligomycin. Furthermore, the cell death induced by rotenone or valinomycin was not apparent until six to eight hours after treatment. Thus, cells over-expressing BCl-x_{L} were more sensitive to antimycin A, but not to other mitochondrial inhibitors.

The effects of BCl-x_{L} over-expression on non-tumorigenic cells was also examined. In particular, the sensitivity of cells that over-express BCl-x_{L} to antimycin A was further examined in the non-tumorigenic mouse liver cell lines, AML-12 (ATCC CRL-2254) and NMH. Briefly, AML-12 cells were transfected as described above with pSFFV.Bcl-x_{L}(sense) and pSFFV.neo. AML-12-pSFFV. Bcl-x_{L}(sense) cells expressed approximately 3 to 4 fold higher BCl-x_{L} protein levels than did AML-12 cells transfected with the control plasmid, pSFFV.neo, when assayed by Western blot analysis. The AML-12.Bcl-x_{L} cells demonstrated increased sensitivity to antimycin A, which is consistent with the results from TAMH cells. Similar results were also found with the mouse liver cell line NMH and with two other TAMH clones stably transfected with a vector that over-expresses BCl-x_{L}. TAMH cells that over-express the related family member protein Bcl-2 were also more sensitive to antimycin A than were control cells.

Thus, cells over-expressing BCl-x_{L} or Bcl-2 exhibited increased sensitivity to antimycin A. In particular, this inhibitor preferentially induced apoptosis in Bcl-x_{L}-over-expressing liver cell lines, confirming that certain mitochondrially active agents can overcome or bypass the anti-apoptotic effect of BCl-x_{L} over-expression. Since over-expression of BCl-x_{L} or Bcl-2 resulted in a decreased apoptotic sensitivity and has been implicated in multidrug resistance in cancer cells and carcinogenesis, this finding has clinical implications. In particular, this difference represents a significant therapeutic window which can be exploited for preferentially inducing apoptosis in cells over-expressing BCl-x_{L} or Bcl-2, while cells not over-expressing Bcl-x_{L} or Bcl-2 are minimally affected.

### Example 2

In this example, various biochemical and biophysical indices associated with antimycin A treatment were examined and correlated with cell death. Specifically, reactive oxygen species (“ROS”) and ATP production were examined soon after initiating antimycin A treatment. Other parameters of mitochondrial function were also measured.

Electrons as reducing equivalents are fed into the mitochondrial electron transfer chain at the level of Coenzyme Q (CoQ) from the primary NAD⁺—and FAD-linked dehydrogenase reaction and are transferred sequentially through the cytochrome chain to molecular oxygen. As discussed above, antimycin A inhibits complex III (CoQH₂-cytochrome c reductase) downstream of CoQ. Complex III serves as an electron transfer station for transfer of electrons from CoQ to cytochrome c. Because CoQ is the major source of ROS derived from the mitochondrial respiratory chain (Turrens et al., Arch. Biochem. Biophys. 237:408-414, 1985), inhibition of complex III often leads to increased ROS formation. The production of ROS in this example was measured by incubating control or antimycin A-treated cells with dihydroethidium. ROS present in the sample oxidizes dihydroethidium to the fluorescent product, ethidium (Rothe et aL, J. Leukocyte Biol., 47:440-448, 1990).

Briefly, TABX2S and TABX1A cells were harvested and resuspended at 5×10⁵ cells/ml. These cells were incubated with 5 μM dihydroethidium in tissue culture media for 45 minutes at 37° C. and then submitted for flow cytometric analysis. One hour after antimycin A treatment, when apoptosis was not apparent, the levels of ethidium were increased to a similar extent in both TABX2S and TABX1A cells. Similarly, when peroxide levels were measured by incubating the cells with dichlorodihydrofluorescein (H2-DCF-DA), the increase in peroxide production was the same between the two cell lines. Thus, antimycin A treatment did not stimulate greater formation of ROS in antimycin A-sensitive (TABX2S) cells compared to antimycin A-resistant (TABX1A) cells.

Correlation of ATP production with cell death was examined by comparing the ATP levels in antimycin A-treated cells and control cells treated with DMSO vehicle alone. Similarly treated cells were tested for viability by trypan blue dye exclusion. Mitochondrial ATP production is driven by the electrochemical gradient generated along the respiratory chain. Following complex III inhibition by antimycin A, electron flow is blocked and ATP synthesis is interrupted.

To determine whether there is a negative correlation between ATP production and cell death, TABX2S and TABX1A cells were treated with (1) DMSO, (2) 2 μg/ml antimycin A, or (3) 2 μg/ml antimycin A plus fructose (50 mM added 15 minutes before and 15 minutes after administration of antimycin A). Fructose is a substrate that provides ATP production through the glycolytic pathway. After a 30-60 minute incubation, cells were harvested and intracellular ATP concentrations were determined by an ATP-dependent luciferase-luciferin assay (Sigma, St. Louis, Mo.). The ATP concentrations in DMSO-treated cells were taken as 100%. In parallel experiments, cell viability was determined after six hours.

Intracellular ATP levels were found to decrease by 70 to 75% in both TABX2S and TABX1A cells within 30 minutes of antimycin A treatment. Supplementation with fructose restored the ATP level to approximately 60% of control in both cell lines, but had no effect on subsequent cell death. Thus, ATP levels did not correlate with the extent of apoptosis. For instance, even though there was a higher ATP level in antimycin A-treated TABX2S cells supplemented with fructose than in antimycin A-treated TABX1A cells without fructose, significantly more apoptosis occurred in the former (33% survival vs. 87% survival). These data argue against a primary role of ATP depletion in mediating apoptosis in antimycin A-treated TAMH cells.

To further test if the mitochondrial respiratory chain in cells over-expressing BCl-x_{L} was more sensitive to antimycin A inhibition, cellular respiration was measured by oximetry. Briefly, TABX2S cells (over-expressing Bcl-x_{L}) and control cells (TAMH.neo) were suspended in air-equilibrated complete medium at a density of 3 million cells per milliliter and placed in a thermostatted electrode chamber at 37° C. The cells were treated with 1 μg/ml antimycin A. Polarographic measurements were made with a Clark-type oxygen electrode with continuous recording. Both cell types showed similar reductions in oxygen consumption. At higher concentrations of antimycin A, oxygen consumption was almost completely inhibited in TAMH.neo control cells, while TABX2S cells maintained about 20 percent of basal oxygen consumption. Thus, the sensitivity of cells over-expressing BCl-x_{L} to antimycin A was not a result of heightened effects on ATP levels, ROS generation or cell respiration.

The effect of antimycin A on mitochondrial function was further evaluated with the mitochondrial dye, JC-1 (5,5′,6,6′-tetrachloro-1,1′,3,3′-tetraethylbenzimid-azolcarbocyanine iodide) (Molecular Probes, Eugene, Oreg.), a lipophilic, cationic carbocyanine dye, which has a fluorescence emission at 520 nm (green). JC-1 normally exists in solution as a monomer emitting a green fluorescence. When JC-1 assumes a dimeric configuration (J-aggregate) in a reaction driven by ΔΨₘ, it emits a red fluorescence (Reers et al., Biochem. 30:4480-4486, 1991). The use of JC-1 allows simultaneous analysis of mitochondrial volume (green fluorescence) and ΔΨₘ (red fluorescence). (See Mancini et al., J. Cell. Biol. 138:449-469, 1997.)

Briefly, at 15 and 30 minutes, 5×10⁵ cells were washed, trypsinized and resuspended in 1 ml of growth media. Each sample was stained with 10 μg/ml of JC-1 prepared in DMSO. After 10 minutes of incubation at 37° C., cells were transferred to ice and analysis was performed using a FACScan flow cytometer (Becton Dickinson). The excitation wavelength was 488 nm whereas measurement was performed at 520 and 585 nm for green and red fluorescence, respectively. Green and red fluorescence were measured on FL1 and FL2 channels, respectively. A minimum of 10,000 cells per sample were analyzed. Comparisons were made based on the results of at least three experiments.

There was a clear increase in JC-1 green fluorescence (mitochondrial volume), accompanied by a decline in JC-1 red fluorescence (mitochondrial transmembrane potential) in TABX2S cells one hour after antimycin A treatment. In contrast, JC-1 green and red fluorescence remained relatively unchanged in TABX1A cells. It should be noted that in the control cells (DMSO vehicle-treated cells), neither JC-1 green nor red fluorescence changed after one hour. When earlier time points were examined in TABX2S cells, there was already a significant increase (shift to right) in JC-1 green fluorescence as early as 15 minutes after addition of antimycin A, whereas JC-1 red fluorescence showed little change at this time. This finding suggests that the change of mitochondrial volume precedes that of ΔΨₘ.

The ultrastructural characteristics of TABX2S and TABX₁A cells were further studied by electron microscopy. Briefly, cells were fixed in half strength Karnovsky's fixative and post-fixed in 1% collidine-buffered osmium tetroxide. After dehydration, cells were embedded in Epon 812. Ultrathin sections were stained using saturated aqueous uranyl acetate and lead tartrate and examined using a JEOL 100 SX transmission electron microscope operating at 80 kV. At two hours after exposure to antimycin A, TABX2S cells had become shrunken and displayed chromatin condensation and margination in the nuclei. These data confirm the apoptotic nature of the cell death. The mitochondria were markedly swollen with widening of the cristae, consistent with the increased JC-1 green fluorescence observed previously in this example. JC-1 staining, however, was found to be more sensitive in detecting mitochondrial changes because mitochondrial swelling was not apparent at 30 minutes or one hour when assayed by electron microscopy. The mitochondrial morphology was normal in antimycin A-treated TABX1A (control) cells.

Mitochondrial PT is caused by opening of a large conductance channel in the inner mitochondrial membrane. Opening of a large conductance channel allows free distribution of solutes of less than 1,500 Da and results in dissipation of the proton gradient and osmotic swelling of mitochondria due to the higher solute concentration in the matrix. In isolated mitochondria, the colloid osmotic swelling associated with PT pore opening can be followed by measuring the optical density change at 540 nm (Kantrow et al., Biochem. Biophy. Res. Comm. 232:669-671, 1997). Because antimycin A-treated TABX2S cells demonstrated increased JC-1 green fluorescence by flow cytometry and mitochondrial swelling by electron microscopy, which suggested the occurrence of PT, the effect of antimycin A on osmotic swelling of isolated mitochondria was tested.

Briefly, mitochondria were isolated from TABX2S cultured cells by a modification of the procedure of Maltese et al. (J. Biol. Chem. 260:11524-11529, 1985). Typically, 0.5 to 1×10⁸ cells were harvested and washed once with homogenization buffer (250 mM sucrose, 10 mM Tris-HCl, 1 mM EDTA and 1 mg/ml BSA, (pH 7.4)). The cell suspension was exposed to nitrogen at 250 psi for 30 minutes in a “cell disruption bomb” (Parr, Moline, Ill.) or homogenized in a Dounce homogenizer with a loose-fitting pestle until >90% of cells were broken. The homogenate was centrifuged at 800×g for 10 minutes. The supernatant was removed and centrifuged at 10,000×g for 10 minutes at 4° C. The pellet was resuspended and again centrifuged at 10,000×g for 10 minutes. The mitochondrial pellet thus obtained was resuspended and adjusted to 0.5 mg protein/ml in an isotonic buffer consisting of 100 mM KCl, 75 mM mannitol, 25 mM sucrose, 5 mM Tris-phosphate, 10 mM Tris-HCl (pH 7.4), 0.05 mM EDTA and 5 mM succinate. For light scattering studies (e.g., for measurement of PT), the mitochondrial suspension was placed in a quartz cuvette, and continuous measurements of light absorption at 540 nm were obtained using a PerkinElmer Lambda 2 spectrophotometer.

Antimycin A added directly to the purified mitochondrial preparation at a concentration of 2 μg/ml caused mitochondrial swelling, which was detected by a rapidly occurring drop in absorbance at 540 nm in mitochondria prepared from TABX2S cells. A rapid fall in light absorbance is characteristic of large amplitude swelling. In contrast, mitochondria from TABX1A cells did not exhibit similar permeability changes and swelling, even at much higher concentrations of antimycin A. The addition of 100 mM CaCl₂ resulted in mitochondrial swelling of both TABX2S and TABX1A mitochondria. In contrast to these results with antimycin A, Bcl-x_{L}-expressing mitochondria were moderately resistant to calcium-triggered mitochondrial swelling.

The effects of antimycin A on isolated mitochondia were also tested using the ΔΨₘ-sensitive JC-1 probe. Isolated mitochondria were loaded with JC-1 prior to treatment, and mitochondrial labeling was determined using FACS. Relative to either initial mitochondrial red fluorescent staining or the basal fluorescence intensity of mitochondria treated with an uncoupler, carbonyl cyanide m-chlorophenylhydrazone (CCCP), antimycin A caused a much greater decrease in ΔΨₘ of mitochondria having high levels of BCl-x_{L} (TABX2S) than control mitochondria (TABX1A). Antimycin A-treated mitochondria with high levels of Bcl-x_{L} had lower levels of JC-1 staining than parallel samples treated with CCCP. Uncoupled mitochondria still retain a significant Donnan potential because of trapped anionic species and it is likely that antimycin-induced PT and/or swelling of mitochondria led to a further reduction of this residual potential. Mitochondria from TAMH.neo cells had an intermediate response to antimycin A.

In summary, examination of mitochondrial characteristics of transfected cells over-expressing BCl-x_{L} in response to antimycin A demonstrated that ATP depletion and increased ROS production, which are direct consequences of complex III inhibition, did not correlate with cell death. Rather, antimycin A induced mitochondrial swelling in cells over-expressing BCl-x_{L}, as demonstrated by the flow cytometry and electron microscopy data discussed above. In addition, the findings that isolated mitochondria over-expressing BCl-x_{L} undergo rapid swelling after addition of antimycin A, while control mitochondria are completely resistant, clearly demonstrated the local effect of BCl-x_{L} in conferring antimycin sensitivity on mitochondria. Thus, antimycin A causes selective cell death by a mechanism independent of its mitochondrial complex III inhibition, but not dependent on BCl-x_{L} protein levels.

### Example 3

This example demonstrates that antimycin A-induced cell death is caspase independent. Bcl-2-like proteins can suppress apoptosis through direct and indirect effects on the cytosolic caspase-activating apoptosome complex (caspase-9, APAF-1 and cytochrome c) or by maintaining mitochondrial membrane integrity and osmotic homeostasis (Cosulich et al., Curr Biol. 9:147-150, 1999). Thus, antimycin A could initiate apoptosis in Bcl-x_{L}-over-expressing cells by inducing BCl-x_{L} to promote, rather than oppose caspase activation, possibly by altering interactions with APAF-1 (Pan et al., J. Biol. Chem. 273:5841-5845, 1998; Hu et al., Proc Nat. Acad. Sci USA. 95:4386-4391, 1998).

TABX2S and TABX1A cells were exposed to the broad spectrum caspase inhibitor, benzyloxycarbonyl-Val-Ala-Asp-fluoromethyl ketone (zVAD-fmk). Antimycin A-induced death of TABX2S cells was found to be caspase-independent, as shown by the inability of zVAD-fmk to rescue such cells from cell death. This result indicates that the pro-apoptotic activity of antimycin A does not require caspase activity.

### Example 4

In this example the ability of antimycin A to promote mitochondrial depolarization in conjunction with BCl-x_{L} expression was tested using a rhodamine 123 (“Rh-123”) retention assay (Petit et al., Eur. J. Biochem. 194:389-397, 1990; Imberti et al., J. Pharmacol. Exp. Ther. 265:392-400, 1993, each incorporated herein by reference). Rh-123 is a cationic lipid-soluble fluorescent dye that accumulates in mitochondria in proportion to the mitochondrial membrane potential. Mitochondria were isolated from TABX2S cells (over-expressing Bcl-x_{L}) and from control cells, prepared as described in Example 2. The isolated mitochondria were loaded with Rh-123 by incubating with 10 μM Rh-123 for 30 minutes, washed and resuspended in buffer. Five minutes after adding antimycin A, or a control diluent, the level of Rh-123 retained by the mitochondria was determined by flow cytometry. Less than 40% of Rh-123 was retained in antimycin A-treated TABX2S mitochondria, compared with greater than 80% retained in control mitochondria. These results indicate that antimycin A induces membrane depolarization, with rapid kinetics, in mitochondria from TABX2S cells, but not in control mitochondria.

### Example 5

To probe a potential interaction between antimycin A and BCl-x_{L}, docking analysis was performed using the crystallographic structure of the BCl-x_{L} protein and antimycin A coordinates from the NMR structure (Muchmore et al., Nature 381:335-341, 1996; Sattler et al., Science 275:983-986, 1997) and the Available Chemicals Directory (Molecular Design, Ltd., San Leandro, Calif.). The program suite, DOCK (Kuntz, Science 257:1078-1082, 1992), was used to determine if there is a compatible site on BCl-x_{L} for binding of antimycin A and, if so, an optimal binding configuration. The DOCK program systematically moves the molecular structure of antimycin A along the surface of the BCl-x_{L} structure and searches for a potential binding site based on shape complementarity, electrostatic interaction, hydrogen bond formation and other chemical energies. An optimal binding site was identified in the BCl-x_{L} structure. Antimycin A was predicted to bind in an extended conformation to the hydrophobic pocket of BCl-x_{L} formed by three conserved domains in the Bcl-2 family, BH1, BH2, and BH3. This binding site overlapped with the dimerization interface for Bak BH3 peptide and BCl-x_{L} previously determined by NMR spectroscopy (Sattler et al., Science 275:983-986, 1997).

### Example 6

Based on the computer modeling prediction that antimycin A could directly bind to the hydrophobic pocket of Bcl-x_{L}, fluorescence spectroscopy was used to detect such a direct interaction. Antimycin A₃ exhibits a fluorescence maximum at 428 nm. The binding of antimycin A₃ to protein causes an increase in fluorescence intensity at the same wavelength.

In this assay, 0 to 5 μM antimycin A₃ (Sigma Chemical Co., St. Louis, Mo.) was added to a physiological buffer (50 mM Tris-HCl pH 8.0, 0.2 M NaCl, 2 mM EDTA, 0.5% v/v glycerol) containing recombinant Bcl-2 or BCl-x_{L} protein under conditions that permitted antimycin A₃ to bind to the BH3-binding domain of Bcl-2 or BCl-x_{L} (22.5° C. on a Hitachi F-2500 fluorescence spectrofluorimeter equipped with a thermostatted cell holder). Bovine serum albumin (BSA), which is known to bind antimycin A, and lysozyme were used as positive and negative controls, respectively. The excitation wavelength was 335 nm, and the maximum emission wavelength for antimycin A₃ was 428 nm with a slit width of 10 nm. The samples were mixed in a quartz cuvette and checked for inner filter effect over the concentration range of antimycin A₃ used for this study. Blanks containing antimycin A₃ at the same concentration as the experimental samples were used as controls in all measurements and necessary background corrections were made.

Recombinant human Bcl-2ΔC22 (a recombinant human Bcl-2 with a delection of 22 amino acid residue membrane anchor sequence from the carboxyl end) and mouse Bcl-x_{L}ΔC20 (a recombinant murine BCl-x_{L} with a delection of the 20 amino acid residue membrane anchor sequence from the carboxyl end) fused with poly-His at the N-terminus were chromatographically purified to homogeneity. The concentrations of antimycin A₃ and stock solutions of recombinant proteins were quantitated using an extinction coefficient of 7.24/mM/cm at 320 nm and by Bradford assay, respectively. The stoichiometric ratio of antimycin A₃ and Bcl-2 producing the maximal change in antimycin A₃ fluorescence was determined with incremental addition of antimycin A₃ to a 1.98 μM solution of recombinant Bcl-2 in a volume of 2.1 milliliters. The change in volume resulting from the addition of antimycin A₃ was less than 5%. For peptide displacement experiments, a solution of 2 μM antimycin A₃ and 3 μM Bcl-2 was allowed to reach binding equilibrium at 4° C. prior to fluorescence measurements. Native peptide corresponding to the BH3 domain of Bak (72-Gly Gln Val Gly Arg Gln LeuAla Ile Ile Gly Asp Asp Ile Asn Arg-87 (SEQ ID NO:1)) or a mutant peptide with a single amino acid change (Leu78Ala-BH3) was added to the solution and the fluorescence measurements were repeated.

The fluorescence of the solution containing recombinant Bcl-2 and antimycin A₃ was increased above the fluorescence of antimycin A₃ alone, indicating that binding had occurred. The fluorescence intensity of antimycin A₃ also increased in the presence of BSA (the positive control), but not in the presence of lysozyme (negative control). The intrinsic fluorescence at 428 nm of antimycin A₃ increases by as much as 18% in the presence of Bcl-2 protein. The maximum change in fluorescence intensity of antimycin A₃ was observed at a molar stoichiometric ratio of antimycin A₃ to Bcl-2 of 1:1, as determined from a Job plot.

The BH3 peptide is also known to bind to the hydrophobic pocket of BCl-x_{L} and Bcl-2. To determine if the site of antimycin A₃ interaction was the hydrophobic pocket of Bcl-2, a competitive binding assay was used. The relative concentrations of antimycin A₃ and Bcl-2 were adjusted to maximize formation of the antimycin A₃:Bcl-2 complex, as indicated by the fluorescence increase of antimycin A₃. BH3 peptide was then added to the preformed antimycin A₃:Bcl-2 complex, as described above. The fluorescence intensity of antimycin A₃ was inversely related to the concentration of BH3 peptide added. At a molar excess of BH3 peptide, antimycin A₃ fluorescence coincided with that of solutions of free antimycin A₃ (without Bcl-2), indicating the displacement of antimycin A₃ from Bcl-2. No overlapping fluorescence was observed from either the BH3 peptide or Bcl-2:BH3 peptide complex, and BH3 peptide alone did not affect antimycin A₃ fluorescence. BH3 peptide displaced antimycin A₃ from Bcl-2 polypeptide with an approximate Michaelis constant of 2.5 μM.

The ability of the mutant Bak BH3 peptide, Leu78Ala-BH3 (L78A-BH3), to displace antimycin A₃ bound to Bcl-2 polypeptide was also tested. The affinity of L78A-BH3 peptide for the BCl-x_{L} hydrophobic pocket is diminished by two orders of magnitude compared to native Bak BH3 peptide. The L78A-BH3 peptide showed significantly reduced ability to displace antimycin A₃ from Bcl-2. Equivalent displacement of antimycin A₃ occurred at a forty fold higher concentration of L78A-BH3 peptide than that required for the native Bak BH3 peptide, which demonstrated the specificity of antimycin A₃-binding to the hydrophobic pocket of Bcl-2. The displacement of antimycin A₃ from BCl-x_{L} similarly required much higher concentrations of the L78A BH3 peptide. These results are consistent with the docking model in which antimycin A₃ is predicted to bind to Bcl-x_{L} at the same binding site as the BH3 peptide the hydrophobic pocket.

### Example 7

The effects of antimycin A in TABX2S cells are similar to the reported mitochondrial and pro-apoptotic effects of peptides derived from the BH3 domain of Bax-like proteins (Chittenden et al., EMBO J. 14:5589-5596, 1995; Cosulich et al., Curr Biol. 7:913-920, 1997; Holinger et al., J. Biol. Chem. 274:13298-13304, 1999). Based on this observation, the Bak-derived BH3 peptide was tested to determine if it also selectively depolarized mitochondria from TABX2S cells (over-expressing Bcl-x_{L}).

In this experiment, the synthetic 16-residue Bak BH3 peptide (Example 6) was added to mitochondria from TABX2S cells (over-expressing Bcl-x_{L}) and to control cells. The addition of the Bak BH3 peptide at 3.5 μM induced similar Rh123 dye leakage by TABX2S mitochondria as that produced by antimycin A. Mitochondria from TABX1A cells were minimally affected by the same concentration of BH3 peptide, or by antimycin A. Thus, antimycin A acts like Bak BH3 peptide in inducing membrane depolarization. Although high levels of BCl-x_{L} maintain mitochondrial integrity in intact cells or isolated organelles exposed to a wide range of stressors, the addition of antimycin A or Bak BH3 peptide overcomes this resistance to depolarization. In contrast, the control cells, which express BCl-x_{L} at physiological levels, were resistant to BH3 peptide-induced membrane depolarization.

This dichotomy can perhaps best be explained by the specific interaction of pro-apoptotic BH3 peptides with the hydrophobic groove in the Bcl-x_{L} structure (Sattler et al., Science 275:983-986, 1997). Reduced levels of BCl-x_{L} result in a lower number of binding sites for BH3 peptides and resistance to BH3 -mediated effects. A similar mechanism may explain the specific effects of antimycin A on Bcl-x_{L}-expressing mitochondria. These results suggest that antimycin A acts as a molecular mimic of endogenous pro-apoptotic proteins. Low expression of BCl-x_{L} reduces the mitochondrial toxicity of both antimycin A and BH3 peptide.

### Example 8

In this example the ability of antimycin A to prevent pore formation by BCl-x_{L} was tested. The BCl-x_{L} protein has reversible pore-forming activity. Recombinant human BCl-x_{L} lacking the C-terminal 20-residue membrane anchor sequence, Bcl-x_{L}ΔC20, forms pores in large unilamellar vesicles. A reporter, calcein, can leak out of the vesicles through these pores. If antimycin A affects BCl-x_{L}AC20 pore formation, the leakage of calcein will change, as can be measured by a change in fluorescence.

Large unilamellar vesicles composed of 60% dioleoylphosphatidylcholine and 40% oleoylphosphatidylglycerol were prepared by the extrusion method of Mayer et al. (Biochim. Biophys. Acta 858:161-168, 1986, incorporated herein by reference in its entirety). Briefly, a dry film of lipid was resuspended in an aqueous solution containing 40 mM calcein (Molecular Probes, Eugene, Oreg.), 25 mM KCl and 10 mM HEPES (pH 7.0). After 5 freeze-thaw cycles, the lipidic solution was extruded through 2 Nucleopore filters, 0.1 μm pore diameter. Nonencapsulated material was removed from the vesicles using a SEPHADEX G-50 column (Pharmacia, Uppsala, Sweden), with 10 mM HEPES (pH 7.0), 100 mM NaCl, as the elution buffer. The size of the vesicle suspension was measured by a Coulter N4 Plus-Sizer to confirm that the mean diameter of the vesicle sample was close to the expected size (100 nm). The osmolalities of all solutions were measured in a cryoscopic osmometer (Wescor Inc., Logan, Utah) and adjusted to 0.21 Osmol/kg by the addition of sodium chloride, as necessary. Lipid concentration was measured as described previously (Stewart, Anal. Biochem. 104:10-14, 1989, which incorporated herein by reference in its entirety).

Calcein leakage was determined by adding 2-4 μg of purified Bcl-x_{L}ΔC20 (5 μg/ml, 161 nM) to a solution of 100 mM NaCl, 10 mM HEPES (pH 5.0) containing the large unilamellar vesicles (50 μM final lipid concentration) described above. Changes in the fluorescence intensity were measured in an Aminco-SLM spectrofluorimeter. BH3 peptides and antimycin derivatives were incubated with BCl-x_{L} for 5 minutes prior to addition to the liposome suspension. Assays were performed at 37° C. in a thermostatted cuvette with constant stirring. Excitation and emission wavelengths for calcein were 495 nm and 520 nm, respectively, at a slit width of 4 nm. The 100% fluorescence level for leakage was obtained by detergent lysis (0.1% Triton X-100) of the vesicles containing entrapped calcein.

In vesicles preloaded with calcein, about 40% of the reporter leaks from the vesicles within about 3 minutes of Bcl-x_{L}ΔC20 addition. Leakage of calcein was inhibited in a dose-dependent fashion by antimycin A. At a concentration of 12 μM, antimycin A completely blocked BCl-x_{L} pore-forming activity.

The ability of the Bak BH3 peptide to inhibit leakage of calcein was also tested. Native BH3 peptide inhibited Bcl-x_{L}-induced calcein efflux from synthetic liposomes, with 50% inhibition at about a 20:1 molar ratio of Bak BH3 peptide:Bcl-x_{L} protein. This inhibition is equivalent to the approximately 20:1 molar ratio of antimycin A:Bcl-x_{L} that is required to achieve a 50% inhibition of calcein leakage. In contrast, the mutant L78A-BH3 peptide has a minimal effect on Bcl-x_{L}-induced pore formation even at a 100-fold molar excess. Thus, antimycin A is capable of blocking the ability of BCl-x_{L} to act as a membrane pore.

### Example 9

Studies of cellular respiration, ATP levels and reactive oxygen species in antimycin A-treated cell lines strongly suggested that the observed differences in cell viability could not be explained by the known effects of antimycin A on mitochondrial electron transfer or oxidative phosphorylation. To definitively address whether the Complex III inhibitory activity of antimycin A is involved in the selective death of cells over-expressing BCl-x_{L}, a structure-activity relationship for antimycin A₃ was determined.

In this example, two derivatives of antimycin A₃ were prepared, antimycin A₃ methyl ether (2-methoxy ether antimycin A₃) and phenacyl ether antimycin A₃. The structure of antimycin A₃ was shown above (formula (I), where R₁ is a butyl group). (See also van Tamelen et al, J. Am. Chem. Soc. 83:1639, 1961). Antimycin A₃ methyl ether has the following formula (XIV) and an absolute configuration of [2R, 3R, 4S, 7S, 8R]:

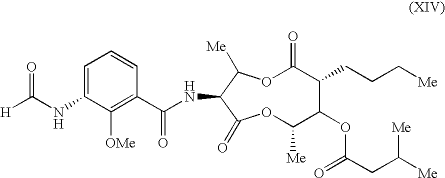

Antimycin A₃ methyl ether is prepared directly from antimycin A₃ as follows: Briefly, antimycin A₃ (14.0 mg) was dissolved in ethyl ether and a stream of diazomethane was passed through the reaction mixture until the yellow color persisted. The reaction mixture was treated with acetic acid until it became colorless. The mixture was reduced to dryness under reduced pressure and chromatographed on a silica gel to yield 14.3 mg of antimycin A₃ methyl ether. The resulting product was characterized by NMR, infrared spectroscopy and mass spectroscopy.

The phenacyl ether derivative of antimycin A₃ was prepared as follows: A solution of antimycin A₃ (5.7 mg, 10.95 mmol) in dry acetonitrile was treated with phenacyl bromide (4.4 mg, 21.9 mmol) and powdered potassium carbonate (6.0 mg, 43.8 mmol). The mixture was allowed to stir at room temperature for 18 hours. The reaction mixture was applied directly to a silica gel chromatography column. The product was eluted with 20% ethyl acetate/hexane to yield 5.4 mg (78%) of the product as a colorless oil. The resulting product was characterized by NMR, infrared spectroscopy and mass spectroscopy.

### Example 10

The antimycin A₃ methyl ether derivative prepared in Example 9 was studied to determine its affect on the apoptotic pathway in cells over-expressing BCl-x_{L}. The methyl ether derivative was previously shown to be inactive as an inhibitor of cytochrome bcl. (See, e.g., Miyoshi et al., Biochim Biophys Acta 1229:149-154, 1995; Takotake et al., Biochim Biophys Acta 1185:271-278, 1994.) The methyl ether also has a negligible effect on cellular O₂ consumption compared to the original antimycin A₃ compound. TABX2S (over-expressing Bcl-x_{L}), TAMH.neo (control) and TABX1A (antisense) cell lines were treated with 2-methoxy antimycin A₃ (2-OMe antimycin A₃, 2-OMeA₃). This derivative exhibited selective cytotoxicity for cells over-expressing BCl-x_{L}, but not for control cells. This pattern was identical to that seen with antimycin A₃, indicating that inhibition of cellular respiration by antimycin was not required for Bcl-x_{L}-related apoptosis.

To confirm these data, assays were also performed with mitochondrial fractions from each cell line using the mitochondrial probe JC-1. Mitochondria from cells over-expressing BCl-x_{L} (TABX2S cells) were strongly depolarized after addition of the 2-methoxy derivative at a concentration of 2 μg/ml. As observed for the parent compound, antimycin A₃, mitochondria with normal levels of BCl-x_{L} expression were not affected by the 2-methoxy analog.

Finally, the 2-methoxy antimycin A₃ derivative was shown to bind recombinant Bcl-2 protein. The 2-OMe antimycin A₃ derivative is non-fluorescent due to the additional electrophilic substituent on the benzene ring. Thus, binding of 2-OMe antimycin A₃ to the Bcl-2 protein can be measured in a competitive binding assay by monitoring fluorescence from antimycin A₃. For these experiments, antimycin A₃ (2 μM) and either 2-methoxy ether antimycin A₃ or phenacyl ether antimycin A₃ (2 μM) were added simultaneously to Bcl-2 polypeptide (3 μM) and allowed to equilibrate for 7.5 minutes at 22.5° C. before measuring the fluorescence intensity of antimycin A₃. The fluorescence of a prebound antimycin A₃-recombinant Bcl-2 complex decreased exponentially with the addition of 2-OMe antimycin A₃, indicating competition for the antimycin A₃ binding site on Bcl-2. As an additional control for binding specificity, the effect of the phenacyl ether derivative of antimycin A₃ was also tested. Although of similar hydrophobicity, the phenacyl ether derivative did not displace antimycin A₃ from Bcl-2. This was likely due to the bulkyness of the phenacyl group which would not be expected to fit into the hydrophobic pocket. These results strongly suggest that the cellular and mitochondrial sensitivity to antimycin A₃ in BCl-x_{L} expressing cell lines results from direct binding of antimycin A₃ to BCl-x_{L} protein. Furthermore, the 2-methoxy ether antimycin A₃ derivative inhibited BCl-x_{L} pore formation in a liposome permeability assay almost as well as antimycin A₃.

The results demonstrate that the antimycins have two structurally distinguishable protein-binding activities, one for binding to cytochrome bc₁, and the other for binding to Bcl-2 family member proteins, and that these activities are separable.

### Example 11

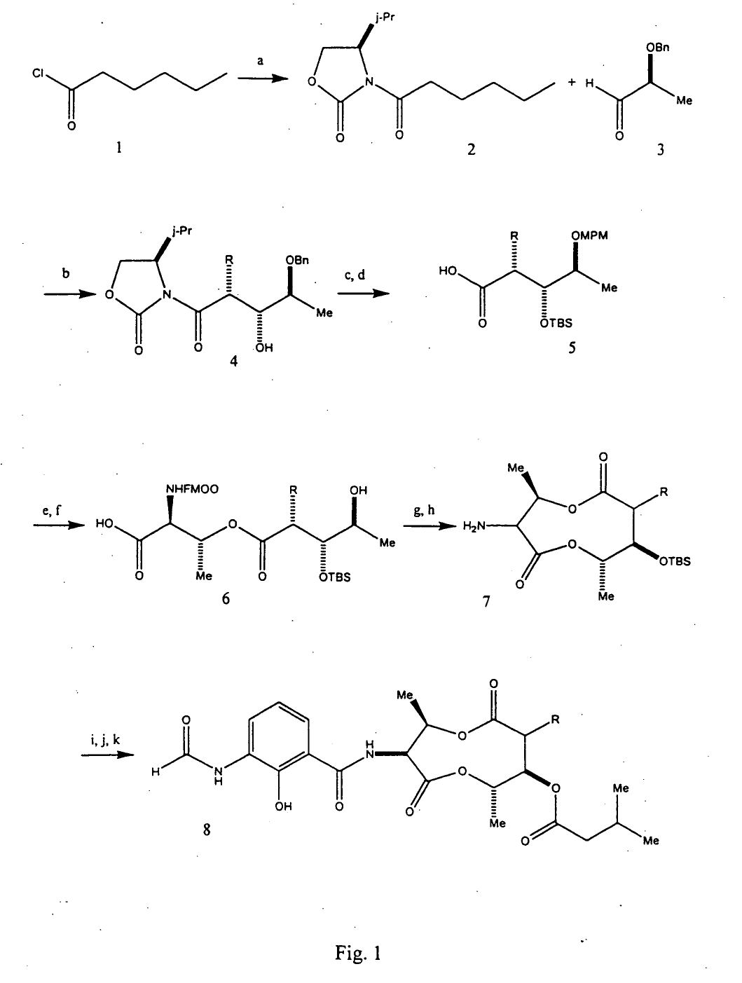

The total synthesis of antimycin A₃ is carried out essentially as described by Shimano for the related dilactones UK-2A and UK-3A (Shimano, Tetrahedron 54:12745-12774, 1998). Briefly, antimycin A₃ is composed of three structural units: an N-formyl-3-aminosalicylic acid, L-threonine and 2-butyl-3,4-dihydroxypentanoic acid. Of the three structural components, N-formyl-3-aminosalicylic acid and L-threonine are commercially available. The dihydroxy pentanoic acid is prepared in a four-step reaction sequence starting with caproyl chloride. Referring to FIG. 1, caproyl chloride is reacted with the Evans valine-derived oxazolidinone, (R)-4-isopropyloxazolidin-2-one, and n-butylLi (Step a). The resulting adduct (2) is reacted by aldol condensation with a chiral aldehyde derived from (S)-(−)-lactic acid (3) in the presence of dibutyl-BOTf and triethylamine (Step b). The 4-hydroxyl group of the resulting adduct (4) is protected as a t-butyldimethylsilyl ether (using TBS chloride and DIEA), followed by peroxide-mediated hydrolysis (using hydrogen peroxide and lithium hydroxide) of the chiral auxiliary to yield the differentially protected dihydroxy pentanoic acid (5) (Steps c and d). Differential protection of the two secondary alcohols allows for the incorporation of various carboxylic acids at the 3 position of the lactone. The carboxylic acid is coupled to N-FMOC-L-threonine benzyl ester with BOP-chloride and DMAP (Step e). Removal of the two benzyl protecting groups with H₂ and Pd/O will yield the dilactone seco-acid (6) (Step f). Lactonization occurs using a BOP-Cl mediated ester-forming reaction with DMAP (Step g). Diethylamine is used to remove the FMOC protecting group to yield the dilactone (7) (Step h). N-formyl-3-amine salicylic acid is coupled to the dilactone using standard carbodiimide chemistry (Step i). In particular, the dilactone is combined with N-formyl-3-aminosalicylic acid using EDCl and HOBT, followed by treatment with TBAF. The final elaboration of the derivatized antimycin A₃ structure is accomplished by fluoride-mediated removal of the silyl protecting group and coupling of the desired acid chloride (e.g., isobutyryl chloride and DIEA) (Steps j and k).

### Example 12

To prepare derivatives of antimycin A₃ that are modified in the isovalerate moiety (i.e., R₂) of the dilactone, the total synthesis of antimycin A₃ (as described in Example 11) is conducted with the following modifications: After dilactonization, the isovaleryl chloride can be substituted by another acyl chloride, such as acetyl chloride, butyryl chloride, and the like.

### Example 13

To prepare derivatives of antimycin A₃ in which the isovalerate moiety (i.e., R₂) of the dilactone is replaced with a hydroxyl group, the total synthesis of antimycin A₃ (as described in Example 11) is conducted up to the last step, at which step the addition of the acyl chloride is omitted.

### Example 14

To prepare derivatives of antimycin A₃ in which the butyl group (i.e., R₁) on the dilactone is substituted with another R group, the total synthesis of antimycin A₃ (as described in Example 11) is conducted with the following modifications: The caproyl chloride of step 1 can be substituted with another acyl chloride, such as propionyl chloride or another linear or branched acyl chloride.

### Example 15

To prepare derivatives of antimycin A₃ in which the 8-methyl group (i.e., R₆) on the dilactone is substituted with another R group, the total synthesis of antimycin A₃ (as described in Example 11) is conducted with the following modifications: The N-FMOC-L-threonine benzyl ester of step 6 is substituted with an N-FMOC-L-serine benzyl ester.

### Example 16

To prepare derivatives of antimycin A₃ in which the 8-methyl group (i.e., R₆) on the dilactone is substituted with a different R (C₂-C₆) group, the total synthesis of antimycin A₃ (as described in Example 11) is conducted with the following modifications: The N-FMOC-L-threonine benzyl ester of step 6 is substituted with an N-FMOC-α-amino-β-hydroxy (C₂-C₆) carboxylic acid benzyl ester (e.g., for C₂: 2-amino-3-hydroxy pentanoic acid benzyl ester).

### Example 17

To prepare a derivative of antimycin A₃ in which the butyl and isovalerate groups (i.e., R₁ and R₂) are replaced with a benzene ring, the total synthesis of antimycin A₃ (as described in Example 11) is modified as follows: N-FMOC L-threonine methyl ester is coupled with the tert-butyl-dimethylsilyl ether of 2-hydroxymethyl benzoic acid in the presence of EDCI and disopropylethylamine. The resulting ester is treated with diethyl amine to liberate the alpha-amino group. The N-formyl-amino salicylic acid is attached using standard carbodiimide chemistry. The threonine methyl ester and the silyl group are removed by treatment with aqueous base. The resulting hydroxyacid is lactonized using carbodiimide chemistry.

The resulting compound has the following formula (XV):

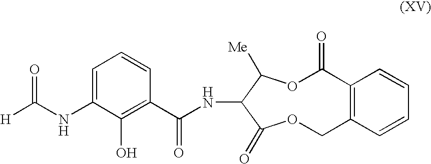

### Example 18

Stability of 2-Methoxy Antimycin Derivatives

The following example determines the stability of certain 2-methoxy antimycin derivatives in an assay for the concentration of the derivatives in plasma samples from rats, dogs, and humans. Similar assays were carried out using culture media and cell extracts. Pharmacokinetics of the 2-methoxy antimycin derivatives in a murine model were also examined.

Materials

All chemicals and solvents used for sample preparation and HPLC analysis were analytical grade. Acetonitrile, methanol, and phosphoric acid were purchased from Fisher Scientific (Atlanta, Ga.). Hepatic S9 fractions from male CD2F1 mice, male Sprague Dawley rats, male dogs, and male humans were purchased from In Vitro Technologies (Boston, Mass.). The National Cancer Institute provided samples of the test compounds and male CD2F1 mice. Fischer 344 rats with implanted jugular cannulas were obtained from Charles River Laboratories (Cambridge, Mass.). All animal use and care procedures were approved by the Institutional Committee on Animal Use and Care at the University of Alabama at Birmingham and conformed to the guidelines found in Guide for the Care and Use of Laboratory Animals, National Academy Press, 1996.

Analytical Method

For analysis of samples, two volumes of cold methanol-acetonitrile (50:50, v/v) were added to portions of plasma, culture medium, or cell extract. The preparations were mixed and then centrifuged to obtain clear supernatants, which were dried at room temperature by compressed air. The residues were dissolved in the mobile phase, and a portion was injected onto the HPLC column.

The HPLC equipment consisted of a Hewlett Packard 1050 ChemStation with a fluorescence detector (Agilent 100 series). Quantification of the test compounds was achieved with a Zorbax SB-18 (5 μm, 150×4.6 mm) analytical column protected by a LiChioCART 100 RP-18 guard column. The mobile phase was methanol:ammonium acetate buffer (87:13 v/v; 0.2 M; pH 5.8, adjusted with HCl). The flow rate was 1 ml/min. The column elute was monitored fluorometrically at an excitation wavelength of 232 nm and an emission wavelength of 418 nm. The amounts of test compounds present in samples were determined from the peak areas.

In the investigated concentration range of 10 and 500 ng/ml, the calibration curves were linear, with mean correlation coefficients (r²) of >0.999. Intra-day and inter-day variations were acceptable. This procedure proved to be adequate for measurement of 2-OMe antimycin A₁ and its analogs in plasma at concentrations >5 ng/ml.

Stability in Plasma

Samples of mouse, rat, dog, and human plasma were incubated at 37° C. with and without addition of NaF (2.5 mg/ml) and EDTA (1 mg/ml). To these preparations, 2-OMe antimycin A₁, 2-OMe antimcyin A₂, 2-OMe antimycin A₃, or 2-OMe antimycin A₅, dissolved in methanol, was added to final concentrations of 500 ng/ml. Portions of plasma, taken at selected times after addition of the 2-OMe antimycin analog, were extracted and analyzed.

Similarly, the stability of 2-OMe antimycin A₁, 2-OMe antimycin A₂, 3-OMe antimycin A₃, and 4-OMe antimycin A₅ in the plasma from mice and humans was evaluated at −80° C. From these preparations, portions were analyzed immediately; other portions were stored at -80° C. and analyzed after 1, 2, and 4 weeks.

Stability in Cell Culture

HL60 and RPMI-8226 cells were maintained in cell culture medium in the presence or absence of 20% fetal bovine serum. 2-OMeA₁ (500 ng/ml) was added to a culture containing 200,000 cells/ml, and to another flask containing only the medium. From the flasks, portions were removed at various times and, as appropriate, centrifuged to collect cells. Samples of cell-free medium, to which NaF/EDTA was added, were extracted. Collected cells were suspended in phosphate-buffered saline (PBS) containing NaF/EDTA, subjected to sonication, and then extracted and analyzed.

S9 Metabolism

To evaluate metabolism of the test compounds by S9 fractions from various species, reaction systems maintained at 37° C. contained 2-OMe-A₃ (500 ng/ml) and S9 (1 mg protein/ml) in 100 mM Tris buffer (pH 7.4). At selected times, triplicate portions of the mixtures were removed and processed for analysis, as described above.

Binding to Plasma Proteins

Solutions of 2-OMe antimycin A₃ were diluted with plasma (dog or human) containing NaF+EDTA to final concentrations of 1.0 or 0.2 μg/ml. Controls were diluted with water to the same final concentrations. These preparations were incubated at 37° C. for 20 min, at which time portions were taken and placed in the sample reservoirs of Amicon Centrifree® ultrafiltration systems (Millipore Co., Bedford, Mass.). At 37° C., the filter systems were centrifuged until the reservoirs were dry. From each filtrate, portions were taken for analysis by HPLC. The amounts present were designated as “free drug” (F). The concentrations of the unfiltered solutions were also determined by triplicate analyses. This amount represented the “total drug” concentration (T). The percentage of 2-OMe antimycin A₃ bound to plasma proteins was calculated by the following formula:
% bound=((T−F)/T)×100

Similar experiments were performed for 2-OMe antimycin A₃ in dog or rat plasma in the absence of NaF/EDTA.

Pharmacokinetic Evaluation

To determine the pharmacokinetics of 2-OMe antimycin A₁ in male CD2F1 mice, groups of three were dosed either intravenously (tail vein) or intraperitoneally with 2-OMe antimycin A₁ (20 mg/kg) in a vehicle of Cremaphor (5%) and ethanol (5%) in 0.9% saline. For both dosing routes, the volume administered was 5 ml/kg body weight. At various times, samples were collected by cardiac puncture into tubes containing NaF/EDTA (dry powders).

Male Fischer 344 rats (five/group) were dosed via a lateral tail vein with either 2-OMe-A₁ or 2-OMe-A₃ (0.25 mg/kg) administered as a 1-min intravenous push. The dosing vehicle was the same as that used for mice. The dosing volume was 1.3 ml/kg. Blood samples were collected and analyzed. Further, samples of plasma, urine, livers, and kidneys were collected from rats kept in metabolism cages for 24 hr. To retard the breakdown of the test compounds, NaF+EDTA was present in the tubes used for collecting blood and urine and was added to tissue homogenates.

Pharmacokinetic values were derived with WinNonlin® software (Mountain View, Calif.).

Results

Stability in Plasma

In the absence of NaF/EDTA at 37° C., none of the compounds was notably stable at 37° C. in either type of plasma. After 60 min, 61% of the added 2-OMe-Al and 53% of the added 2-OMe-A₂ remained in dog plasma. For the other compounds and other types of plasma, however, <33% of the amounts added were present at this time.

In the presence of NaF/EDTA at 37° C., 2-OMe-A₁ was substantially stable for 60 min in mouse and rat plasma, moderately stable in human plasma, and unstable in dog plasma. NaF/EDTA had relatively little effect on the stability of 2-OMe-A1 in dog plasma. 2-OMe-A₂ was generally stable in all types of plasma, the greatest decrease in concentration (18%) was noted for human plasma. 2-OMe-A₃ was largely stable in dog and rat plasma, but there were notable decreases in concentration for human and mouse plasma. 2-OMe-A₅ was stable in human and dog plasma, but, in 60 min, there were notable decreases in concentration for mouse and rat plasma.

In the absence of NaF/EDTA at −80° C., none of the compounds was completely stable over 4 weeks in either type of plasma. In mouse plasma, 2-OMe-A₁ was most stable. The amounts remaining after 4 weeks were as follows: 2-OMe-A₁, 78%; 2-OMe -A₂, 63%; 2-OMe-A₃, 45%; and 2-OMe-As, 27%. In human plasma, 2-OMe-A₁ was also most stable. The amounts remaining after 4 weeks were as follows: 2-OMe-A₁, 86%; 2-OMe-A₂, 70%; 2-OMe-A₃, 73%; and 2-OMe-A₅, 79%. In the presence of NaF/EDTA at -80° C., 2-OMe-A₁ was essentially stable in mouse plasma, but the other compounds showed some instability. The amounts remaining after 4 weeks were as follows: OMe-A1, 96%; 2-OMe-A₂, 77%; 2-OMe-A₃, 90%; and 2-OMe-A₅, 63%. In human plasma, 2-OMe-A₁, 2-OMe-A₃, and 2-OMe-A₅ were essentially stable. The amounts remaining after 4 weeks were as follows: 2-OMe-A₁, 93%; 2-OMe-A₂, 83%; 2-OMe-A₃, 95%; and 2-OMe-A₅, 92%.

Stability in Cell Culture

In the medium from cell cultures, 2-OMe-A₁ was notably less stable in the absence of fetal bovine serum. More 2-OMe-A₁ accumulated in cells (or was attached to cells) in the absence of FBS. There was no appreciable difference between results obtained with HL-60 and RPMI-8226 cells.

Protein Binding

In the presence of NaF+EDTA (Table 5), plasma proteins from mice, rats, dogs, and humans extensively bound (>99%) 2-OMeA₁, 2-OMeA₂, 2-OMeA₃, and 2-OMeA₅ present at concentrations of 1000 ng/ml. In the absence of NaF+EDTA, similar results were obtained for 2-OMeA₃ in dog and human plasma.

TABLE 5
Protein binding of 2-OmeA₁, 2-OMeA₂, 2-OMeA₃, and 2-OMeA₅ in
mouse, rat, dog, and human plasma

| Test Compound | Mouse | Rat | Dog | Human |
|---|---|---|---|---|
| 2-OmeA₁ | 99.0 | >99.9 | 99.8 | 99.5 |
| 2-OMeA₂ | 99.7 | 99.9 | 99.9 | >99.9 |
| 2-OMeA₃ | 99.1 | 99.8 | 99.3 | 99.9 |
| 2-OMeA₅ | 99.7 | >99.9 | 99.1 | 99.6 |

S9 Metabolism

In experiments to determine the effect of S9 proteins on the stability of 2-OMe-A₃ (500 or 2000 ng/ml), the initial levels of the compound dropped rapidly, even in the absence of S9 proteins. Thus, 2-OMe-A₃ was not stable in solution at pH 7.4. Under these conditions, mouse, rat, and dog S9 components enhanced the instability of 2-OMe-A₃. At concentrations of 500 ng/ml, the half-life values for 2-OMe-A₃ in the presence of mouse, rat, or dog S9 were greater than 5 min. In the presence of human S9, the half-life was only slightly less than that for the control. At concentrations of 2000 ng/ml, the half-lives for 2-OMe-A₃ in the presence of mouse, rat, or dog S9 were again greater than 5 min. In the presence of human S9, the half-life was about 10 min, which was moderately greater than when no S9 was present.

Pharmacokinetics

Following an intravenous dose of 2-OMe-A₁ (20 mg/kg) to mice, plasma levels dropped rapidly; the concentration at 30 min after dosing (1040 ng/ml) was about 15% of that at 5 min after dosing. The value for t_{1/2} was 19 min. Nevertheless, the drug was still detectable (15 ng/ml) at 24 hr after dosing. With an intraperitoneal dose of 20 mg/kg, plasma levels increased to a maximum (483 ng/ml) at 30 min after dosing, then decreased with a t_{1/2} value of 183 min. The drug was still detectable (18 ng/ml) at 24 hr after dosing. Relative to dosing intraperitoneally, the intravenous dose gave higher AUC and Cₘₐₓ values and lower values for t_{1/2}, MRT, and Vₛₛ (Table 6). The clearance was similar for both doses.

For rats dosed intravenously with 2-OMe-A₁ (0.25 mg/kg), plasma levels dropped quickly, with a t_{1/2} value of 2.3 min. For rats dosed intravenously with 2-OMe-A₃ (0.25 mg/kg), plasma levels dropped even more quickly, with a t_{1/2} value of 1.0 min (Table 6). At 2 hr after dosing, little or no 2-OMe-A₁ or 2-OMe-A₃ was detectable. Although the values for Cₘₐₓ and Vₛₛ were similar for 2-OMe-A₁ and 2-OMe-A₃, the AUC and MRT values were lower for 2-OMe-A₃, and its clearance was faster. For these rats, little or no detectable amounts of 2-OMe-A₁ or 2-OMe-A₃ were present in the urine, livers, or kidneys at 24 hr after dosing.

TABLE 6
Pharmacokinetic values for 2-OMe-A₁ following intravenous
or intraperitoneal injection into male CD2F1 mice.

| Parameter | Intravenous dose | Intraperitoneal dose |
|---|---|---|
| Dose (mg/kg) | 20 | 20 |
| AUC (ng · hr/ml) | 3720 | 2410 |
| T_{1/2} (min) | 19.2 | 183 |
| Cₘₐₓ (ng/ml) | 8000 | 483 |
| CL (ml/g/hr) | 5380 | 7340 |
| MRT (min) | 27.6 | 474 |
| Vₛₛ (ml/g) | 2500 | 32400 |

TABLE 7
Pharmacokinetic parameters of 2-OMeA₁ and 2-OMeA₃ following
intravenous injection into Fischer 344 rats.

| Parameter | 2-OMeA₁ | 2-OMeA₃ |
|---|---|---|
| Dose (mg/kg) | 0.25 | 0.25 |
| AUC (ng · hr/ml) | 47 | 18 |
| T_{1/2} (min) | 2.3 | 1.0 |
| Cₘₐₓ (ng/ml) | 970 | 920 |
| CL (ml/g/hr) | 5.48 | 15.0 |
| MRT (min) | 3.3 | 1.4 |
| Vₛₛ (ml/g) | 0.29 | 0.37 |

Results from the present study showed that stability of 2-methoxy antimycin compounds depended on the presence of NaF/EDTA (for esterase inhibition). Although the precise metabolic mechanisms were not determined in this study, the major degradation pathway may be associated with esterase activity in plasma, since addition of NaF/EDTA largely prevented in vitro degradation, regardless of species.

In addition, mouse, rat, and dog S9 components, but not human S9 components, enhance the in vitro instability of 2-OMe-A₃. It is likely that, especially in humans, metabolism of 2-methoxy antimycin A compounds will occur primarily in plasma. The S9 metabolic study indicated that human S9 fractions had minimal metabolic activity. Because of this, there would likely be more intact drug available in humans compared to other species.

The pharmacokinetic studies in mice indicated that 2-OMe-A₁ had a short plasma half-life with a relatively large volume of distribution after intravenous administration and a prolonged plasma half life and greater volume of distribution after intraperitoneal administration. Also, a pharmacokinetic study in rats, involving a much lower dose administered intravenously, showed that there were no appreciable differences between 2-OMe-A₁ and 2-OMe-A₃.

### Example 20

Anti-Tumor Activity of 2-Methoxy Antimycin Compounds

In this example the in vivo tumor activity of antimycin A or a 2-OMe antimycin A were tested. Briefly, the following procedure was followed.

Cell Culture

The RPMI-8226 cell line was supplied by W. Dalton (Univ. Arizona). U266 and H929 cell lines were obtained from the American Type Culture Collection (Rockville, Md.). Cell lines were grown in RPMI 1640 (Gibco, Grand Island, N.Y.) supplemented with 5% fetal bovine serum (Hyclone, Logan, Utah). Normal bone marrow samples were obtained from allogeneic transplant donors at the Fred Hutchison Cancer Research Center, Seattle, Wash., with appropriate patient consent and Internal Review Board approval. Primary cells were maintained in Iscove's medium (Gibco) supplemented with 10% bovine calf serum, 100 ng/ml stem cell factor (Amgen, Thousand Oaks, Calif.), and 50 ng/ml interleukin-3 (Biosource, Camarillo, Calif.).

GI₅₀ Assays

RPMI 8226 cells were grown in 96 well plates for 24 h prior to addition of 2-methoxy Antimycins. Antimycin stocks in DMSO were serially diluted in RPMI media. Cells were incubated for 48 h with compounds at final compound concentrations ranging from 10⁻⁴ to 10⁻⁸ M. MTT assays were performed in quadruplicate and percent growth inhibitions were calculated as (A₅₇₀ (control cells)—A₅₇₀ (treated cells))/A₅₇₀ (control cells). The GI50 was extrapolated from semi-log plots of the dose response for each compound. Alternatively, cells were plated in 96-well round-bottomed microplates in complete medium, to which various doses of antimycin A or 2-OMe antimycin A were added 12 to about 16 h later, in triplicate. After 72 h drug exposures, 1 μCi/ml [³H]-thymidine was added to wells. Cells were incubated for an additional 24 h, and then transferred to GF/C filter plates (Packard) using a plate washer. Filters were dried, added to scintillant, and counted in a TopCount scintillation counter (Packard). Drug response was expressed as the percentage of vehicle-treated controls, and each value shown as the average of three determinations.

Flow Cytometry

Cell survival was assayed by exclusion of propidium iodide (PI; 10 μg/ml) by unfixed cells. Annexin V-FITC (Pharmingen) or 3, 3′-dihexyloxacarbocyanine iodide (DiOC₆(3); Molecular Probes) staining was analyzed together with PI to discriminate early apoptotic cells. Apoptotic cells were also measured as sub-G1 events among ethanol-fixed cells stained with 10 μg/ml PI. All cell samples were analyzed using a benchtop FACSCalibur™ (Becton Dickinson, San Jose, Calif.). Flow data were analyzed using MultiCycle AV software (Phoenix Flow Systems). For intracellular pH measurements, cell pellets were washed once and resuspended in 2 ml of HEPES-buffered medium (no phenol red and no serum). Carboxy-SNARF-1-AM (Molecular Probes, 1 mM stock in DMSO, stored at −20° C.) was added to a final concentration of 10 μM, and the cells were incubated for 30 min at 37° C. Following incubation with SNARF, cells were sedimented and the pellets were held on ice. Immediately before analysis, pellets were resuspended in Earle's balanced salt solution (HBSS; experimental buffer) or high-[K+] buffer containing nigericin (calibration samples).

The analysis by flow cytometry (Becton Dickinson FACScan™) was done with excitation at 488 nm, and emission at 585 and 640 nm (corresponding to the H⁺-bound and—free forms of carboxy-SNARF-1-AM, respectively). Determination of the number of cells in the various populations was performed by drawing regions on the profiles generated by analyzing pH calibration samples. The calibration samples were generated by incubating untreated cells with SNARF in high potassium buffers (20 mM NaCl, 130 mM KCl, 1 mM MgCl₂, 1 mM CaCl₂, 5 mM HEPES; titrated to the appropriate pH with HCl and NaOH) containing nigericin (Sigma, 1 mM stock in absolute ethanol, stored at 4° C.) at 10 μM final concentration and fixed pH ranging from 6.5 to 8.5 (nigericin was added after the pH titration).

Microsequential Injection-Lab On Valve Studies

Detailed methods have been previously published (Schulz et al., Analyst 127:1583-1588, 2002). Cytopore™ beads (Amersham Pharmacia Biotech, Upsala, Sweden) were hydrated in Hanks balanced salt solution (Gibco-BRL, Grand Island, N.Y.) and autoclaved according to manufacturers instructions. Beads were incubated in serum-containing media overnight. TAMH cells were transferred to the bead slurry at an approximate ratio of 50 cells per bead and grown in spinner culture flasks with gentle stirring. Beads were collected for metabolic studies at cell densities of approximately 100-500 cells/bead. For experiments, cell medium was replaced with HBSS.

Studies were carried out using a FIAlab® 3000 automated sequential injection analyzer (FIAlab® Instruments, Medina, Wash.), consisting of a 6-position lab-on-valve (LOV) manifold controlled by a precision bi-directional syringe pump. A 6 port multiposition valve (MPV) with a dedicated syringe pump was added as a bioreactor module. FIAlab® software version 5.0 was used to control all of the system components and for data collection and analysis. The flow cell in the LOV was illuminated by a long wave UVA pencil light (Spectronics Corp., Westbury, N.Y.) with a 600 μm UV fiber optic connection to a CCD spectrophotometer (Ocean Optics, Dunedin, Fla.). The entire apparatus was placed inside an incubator set at 37° C.

Glucose and lactate concentrations were assayed as substrates in first-order NAD-linked enzymatic reactions, with NADH generation monitored by absorbance at 340 nm. Infinity™ glucose reagent, glucose and lactate standards, and bovine heart lactate dehydrogenase (LDH) (Sigma, St. Louis, Mo.) were prepared fresh daily. The glucose reagent included final concentrations of >1500 U/I hexokinase, >3200 U/1 glucose-6-phosphate dehydrogenase, 2.1 mM ATP and 2.5 mM NAD⁺. The lactate reagent contained final concentrations of 2000 U/ml LDH and 2.5 mM NAD⁺ in glycine buffer.

An assay cycle is initiated by packing a column of cells attached to beads in the microbioreactor, which is upstream of the LOV flow cell. The cells-on-beads were perfused with 2-OMe antimycin A in HBSS, followed by a stop flow period (120 s) to allow depletion of glucose and accumulation of lactate in the interstitial volume of the microbioreactor. After the stop flow period, 3 μl of the interstitial fluid from the cell collumn is injected through to the LOV flow cell previously loaded with glucose or lactate reagent and 340 nm absorbance recorded for 30 s after mixing. Calibration standards were used to convert endpoint absorbances to concentrations. Each data point represents the mean of three independent measurements done on new columns of TAMH cells-on-beads.

Mitochondrial Membrane Potential Measurements

RPMI-8226 cells (2×10⁷) were resuspended in buffer containing 130 mM KCl, 5 mM malate, 5 mM glutamate, 2 mM KPO4, 5 mM HEPES, pH 7.0 with 5 μM safranin O dye, in a stirred cuvette at 28° C. Fluorescence was measured at 495 nm excitation, 586 nm emission on a RF-5301PC spectrofluorophotometer (Shimadzu, Japan). Safranin O fluorescence was quenched following addition of 0.0025% digitonin as the dye accumulates in active mitochondria (Fiskum et al., Methods Enzymol. 322:222-234, 2000). Depolarization of the mitochondrial inner membrane caused a shift of safranin O localization from mitochondria to cytoplasm, evident as increased fluorescence.

Metabolic Studies of RPMI-8226 Cells

RPMI-8226 cells were starved for glucose by growing in glucose-free RPMI for 24 h in the presence of 2% dialyzed serum and 2 mg/ml sodium pyruvate. This was followed by continuous treatment for 5 and 24 h with antimycin A₁ at 2.5 μM and 2-OMe antimycin A₁ at 1 μM in the presence of indicated concentrations of glucose in RPMI supplemented with 5% dialyzed serum. The control samples consist of untreated cells, cultured as above and in the presence of 2 mg/ml glucose.

The supernatants from the cells were used for the measurement of glucose concentrations by using a Sigma diagnostic assay kit (Glucose HK assay kit). The manufacturer's procedure was modified, to adapt it to a 96 well plate assay (serial dilution of the glucose standard and samples were made, not to exceed about 30 to 40 μg/well; a maximum of 50 μl of sample was used, in a total volume of 250 μl/well). Data represent micrograms of glucose per 50 μl of supernatant.

Tumor Xenografts

Six to nine-week old Nod/Les2 Scid/J mice were inoculated with 3×10⁷ RPMI-8226 cells by interscapular subcutaneous injection. Mice were maintained under specific pathogen-free conditions. Palpable tumor nodules were measured in two dimensions with calipers and tumor volumes calculated in mm³ as (length×width²)/2. Blood samples were collected by retro-orbital bleed for human light chain measurements by ELISA with lambda-specific antibody and horseradish peroxidase detection (BD Biosciences). Animals were sacrificed by halothane inhalation, and histologic examination of tumors and internal organs was performed. All experiments were approved by the Fred Hutchison Cancer Research Center Institutional Animal Care and Use Committee.

Preparation and in vivo Dosing of 2-OMe Antimycin A

2-OMe antimycin A₁ was synthesized from antimycin A₁ (Sigma) (comprising about 80% antimycin Ala and about 20% A_{1b}) and dissolved in phosphate-buffered saline with 20% Cremaphor, 25% ethanol (3 mg/ml) for parenteral administration by tail vein injection in a total volume of 100 μl. Control mice received injections of Cremaphor/ethanol vehicle.

Tissue Sections

Tissues were fixed in 10% buffered formalin, embedded in paraffin, sectioned and stained with hematoxylin and eosin. TUNEL staining was performed using 0.3 units/ml terminal deoxynucleotidyl transferase and biotinylated dATP with development by avidin-biotin peroxidase method. BCl-x_{L} immunohistochemistry was performed using anti-Bcl-x_{L} antibody (BD Biosciences) followed by biotinylated secondary antibody and peroxidase-labeled ABC reagent (Vector, Burlingame Calif.).

Results

The cytotoxicity of antimycin A₁ in a panel of human hematopoietic cell lines was assessed using propidium iodide (PI) exclusion assays. Each of the tested myeloma cell lines (RPMI-8226, U266, NCI-H929) was sensitive to 5 to about 20 μg/ml antimycin A, as were several leukemia and lymphoma cell lines (DHL4, Daudi, Ramos, Molt4 and Jurkat). RPMI-8226 cell death was demonstrated by 8 h of antimycin A treatment, with significant accumulation of dead cells by 18 to 24 h. In contrast, seven or eight myeloid leukemia cell lines were resistant to 20 μg/ml antimycin A, even after 48 h of treatment. NB4, an acute promyelocytic cell line, was modestly antimycin-sensitive.

Antimycin A- and 2-OMeA-induced cell death of RPMI-8226 myeloma cells occurred by apoptosis, as demonstrated by accumulations of cells with increased annexin V staining (annexin V⁺, PI⁻), sub-G1 DNA content, or reduced mitochondrial membrane potential. Cells treated with antimycin A and 2-OMeA developed classic apoptotic morphologies as recognized by light microscopy, including fragmented nuclei and marginated chromatin.

In [³H]-thymidine incorporation assays, antimycin A caused 50% growth inhibition (GI₅₀) of RPMI-8226, U266, and NCI-H929 myeloma cells at 100 ng/ml, 50 ng/ml, and 200 ng/ml, respectively. IC₅₀ values for 2-OMeA-treated cells were 100-fold higher (15, 5, and 10 μg/ml) than with antimycin A, consistent with the negligible inhibition of oxidative phosphorylation by this compound (See example and Tzung et al., Nat. Cell Biol. 3:183-191, 2001; Rieske, Biochim. Biophys Acta. 456:195-247, 1976). In contrast, cell killing measured as PI uptake, was similar in the three cell lines at antimycin A and 2-OMeA concentrations of 5 and 20 μg/ml.

Oxidative phosphorylation inhibitors at low doses were in general not effective at killing myeloma cells. Only NCI-H929 cells were killed by oligomycin (2-10 mg/ml), an inhibitor of F0/F1 ATPase, and none of the myeloma cell lines were killed by the complex I inhibitor rotenone (0.5-2.5 mg/ml).

To confirm that the 2-OMeA did not act to inhibit respiration at the concentrations effective in in vitro cytotoxicity assays, O₂ consumption was measured in TAMH hepatocyte and RPMI-8226 myeloma cell lines using a Clark electrode. Respiratory rates of both cell lines were maintained through repeated additions of 10 μM 2-OMeA, to a final concentration of 90 μM. In contrast, addition of 10 μM of antimycin A dampened respiration by >90% and acute decreases in O₂ consumption were observed with 1-2 μM antimycin A. Antimycin A has often been used in experimental models of chemical hypoxia. The metabolic response to hypoxia has been shown to be dominated by a shift to glycolytic metabolism characterized by increased rates of glucose uptake and reductive conversion of pyruvate to lactic acid. Real-time glucose consumption and lactate production by TAMH cells grown on microcarrier beads was monitored using a novel microsequential injection-lab on valve (μSI-LOV) system (Schulz et al., Analyst 127:1293-1298, 2002). Antimycin A-treated cells increased glucose uptake and lactate production at doses between 10-100 nM. Unexpectedly, cells treated with 10-100 nM 2-OMeA demonstrated similar increases in glycolytic metabolism within the first 2 min of treatment. Since 2-OMeA has no effect on cellular respiration at these concentrations, 2-OMeA induced aerobic glycolysis in TAMH cells. The glycolytic response to 2-OMeA was proportional to cellular BCl-x_{L} protein expression.

Metabolic responses to 2-OMe antimycin A in RPMI-8226 cells grown in suspension culture were determined. No acute changes in mitochondrial membrane potential were observed with 2-OMe antimycin A, in contrast to the response to low concentrations of antimycin A₁. However, 2-MeO antimycin A treatment stimulated glycolysis in RPMI-8226 cells, assessed as depletion of glucose from the culture media and reduction in intracellular pH. Natural antimycin A is composed of several closely related compounds, with the major components represented by antimycin Al through antimycin A₅ (Dickie et al., J. Med. Chem. 6:424-427, 1963). 2-O-methylated derivatives of antimycin A₁, A₃ and A₅ were tested individually with RPMI-8226 cells (Table 8). Similar growth inhibition (GI50) was observed for 2-OMeA₁, 2-OMeA₂, and 2-OMeA₃, while 2-OMeA₅ was approximately ⅓ as active, suggesting that alkyl R groups at positions 7 and 8 of the dilactone ring have modest effects on activity. Antimycin A₁ and A₂ share an n-butyl substituent at position 8, while A₁ and A₃ have in common an isovaleryl group at position 7 (Dickie et al., J. Med. Chem. 6:424-427, 1963). The compound with the highest activity, 2-OMeA₁, was selected for in vivo evaluation.

TABLE 8
GI₅₀ concentrations for RPMI-8226 cells treated with
2-methoxy antimycin (2-OME antimycin) compounds.

| COMPOUND | GI₅₀ (μM) |
|---|---|
| 2-OMeA₁ | 8.56 |
| 2-OMeA₂ | 12.18 |
| 2-OMeA₃ | 10.22 |

In the TAMH cell lines used to screen for BCl-x_{L} inhibitors, apoptotic response to 2-OMeA₁ was inversely related to chemosensitivity with standard agents, consistent with targeting of different cell death pathways. Current anti-myeloma regimens incorporate multiple drugs with different mechanisms of action. RPMI-8226 cells were treated with 2-OMeA₁ in combination with standard chemotherapeutic agents used in myeloma treatment: etoposide, melphalan, or daunorubicin (Sonneveld and Segeren, Eur. J. Cancer 39:9-18, 2003). Supra-additive killing was observed with suboptimal combinations of 2-OMeA₁ and etoposide or melphalan.

BCl-x_{L} is expressed in normal bone marrow hematopoietic precursors, where it is essential for cell survival (Park et al., Blood 86:868-876, 1995; Motoyama et al., Science 267:1506-1510, 1995). To examine the potential toxicity of 2-OMeA₁ in normal cells, unfractionated human bone marrow cells were treated in vitro with 2-OMeA₁ (5-20 μg/ml) for 24 to 48 h, and cell viability was measured in flow cytometry assays. Primary lymphoid and myeloid bone marrow cell subpopulations, identified in light scatter profiles, were insensitive to 2-OMeA at the doses tested as judged by PI and annexin V staining.

Natural antimycin A is highly lethal in mice with an LD₅₀ of 0.893 mg/kg for a single intravenous dose (Nakayama et al., J. Antibiotics Japan Ser. A. 63-66, 1956). Although 2-OMeA does not inhibit mitochondrial respiration at concentrations tested in vitro, its toxicity in vivo was unknown. In particular, the possibility existed that de-methylation could regenerate the highly toxic parent compound in vivo. NOD/SCID mice were treated with three intravenous 4 doses of 2-OMeA₁ administered at 10 mg/kg on alternate days, and 6 of 6 mice survived without apparent toxicity. Three of six mice died after intravenous administration of 2-OMeA at a dosage of 20 mg/kg. No gross abnormalities were observed at necropsy of treated mice. Therefore, 10 mg/kg dosing was used for testing of in vivo anti-tumor efficacy. To determine whether 2-OMeA, has anti-tumor activity in vivo, a total of 12 NOD/SCID mice in three experiments were inoculated with 3×10⁷ RPMI-8226 human myeloma cells by interscapular subcutaneous injection. In the first experiment, subcutaneous nodules were palpable for seven of eight mice at 4 days after injection, while the remaining mouse had a measurable nodule on day 6, and human lambda light chain was detected by ELISA in serum samples of all mice by day 14. In total, six mice received three intravenous doses of 10 mg/kg 2-OMeA₁ on alternate days starting on day 6. Six control mice received injections of Cremaphor/ethanol vehicle without drug. One mouse each in the treatment group and the control group died shortly after the third injection. Serum levels of human light chain were reduced an average of 87% in treated mice after the third 2-OMeA₁ injection. Five of six mice dosed with 2-OMeA₁ showed tumor regression during treatment, while tumor nodules progressed in all six of the untreated mice. At about 12 to 15 days after the last dose of 2-OMeA₁ regrowth of tumor nodules was noted in the treatment group. A second round of alternate day treatments with 10 mg/kg 2-OMeA, on days 26 through 31 again led to regression of tumor nodules in 6/6 treated mice. A two-way ANOVA of these data revealed a highly significant effect of 2-OMeA₁ treatment (F=66.83, df=1, p<0.0001).

No adverse effects were noted in any of the mice treated with 2-OMeA₁. Delayed treatments of two tumor-bearing control mice with 10 mg/kg 2-OMeA₁ on days 43, 46 and 48 also led to regression of tumor nodules.

Tumor sections taken 24 and 48 h after a single treatment with 10 mg/kg 2-OMeA₁ (intravenous) showed widespread apoptosis with numerous fragmented nuclei. Apoptotic nuclei and fragments were also labeled by TUNEL staining. BCl-x_{L} staining of tumor sections was heterogeneous, similar to the expression of Bcl-2 and BCl-x_{L} in human solid tumors.

Discussion

Recent discoveries of several small molecule BCl-x_{L} inhibitors with cytotoxic activity have revealed two mechanisms of inhibition, both associated with binding to the hydrophobic groove interface. The compounds BH31-1 and BH31-2 bind to the Bcl-x_{L} hydrophobic groove with low micromolar affinity and displace pro-apoptotic peptides/proteins (Degterev et al., Nat. Cell Biol. 3:173-182, 2001). These compounds do not interfere with BCl-x_{L} membrane pore-forming ability. 2-methoxy antimycin A (2-OMe A), a non-toxic analog of the respiratory poison antimycin A, also binds to the BCl-x_{L} hydrophobic groove with low micromolar affinity, but has weak displacement activity for pro-apoptotic peptides bound at this site (Tzung et al., Nat. Cell. Biol. 3:183-191, 2001; Kim et al., Biochemistry 40:4911-4922, 2001). In contrast to the BH31 compounds, 2-OMe antimycin A interferes strongly with BCl-x_{L} pore formation at cytotoxic concentrations.

Methylation of antimycin A at the 2-hydroxyl position of the salicylate ring reduces oxidative phosphorylation inhibitory activity at complex III by 1000-fold. The relative safety of this compound in vivo was evident from its LD₅₀ dose of 20 mg/kg (for a schedule of 3 intravenous doses on alternate days) compared to an LD₅₀ of 0.893 mg/kg (single intravenous dose) for the parent compound (Nakayama et al., J. Antibiotics Japan Ser. A. 63-66, 1956). The predominant toxicities for antimycin A were noted in lung, heart and kidney (Greselin and Herr, J. Agric. Food Chem. U. 22:996-998, 1974). No cellular injury or inflammation was evident in histologic examinations of normal tissues from mice treated with 10 mg/kg 2-OMeA₁. These results suggest that the parent antimycin A molecule is not regenerated from 2-OMeA₁ to a significant extent in vivo. In addition, preliminary LC/MS analyses of plasma collected after administration of 2-OMeA₁ have not demonstrated reformation of the toxic antimycin A₁.

Aerobic glycolysis is a hallmark of cancer cells, commonly referred to as the Warburg phenomenon. Warburg postulated that a mitochondrial oxidative phosphorylation defect was a prerequisite for tumorigenesis, with a more gradual upregulation of glycolysis during progression to neoplasia (Warburg, Science 123:309-314, 1956). Fixed (intrinsic) deficiencies in oxidative phosphorylation have not been identified as a general feature in cancer, however, despite several decades of investigation. More recently, two transcription factors deregulated in cancers, hypoxia-inducible factor-1 and Myc, have been shown to promote a metabolic shift to aerobic glycolysis by transactivation of glycolytic enzymes and glucose transporters (Semenza et al., Novartis Found Symp. 240:251-260, 2001). As 2-OMeA also stimulated aerobic glycolysis without apparently inhibiting oxidative phosphorylation, Bcl-XL may also function as a critical regulator of the balance of oxidative phosphorylation and glycolytic metabolism (Vander Heiden et al., J. Biol. Chem. 277:44870-44876, 2002).

The 2-OMeA-sensitive human myeloma cell line RPMI-8226 was xenografted into immunodeficient mice to test the in vivo anti-tumor efficacy of the compound. RPMI-8226 myeloma cells grown as subcutaneous tumor nodules were sensitive to 10 mg/kg 2-OMeA₁ given intravenously. Regression of both early tumor nodules (<10 mm³) and large nodules (>1000 mm³) was observed within the first week of administering 2-OMeA₁. Regrowth of early tumor nodules was observed by 10 to 14 days after the initial three doses of 2-OMeA₁, but a second round of treatment resulted in a more prolonged response.

Normal tissues expressing BCl-x_{L} include bone marrow, kidney, and lymphoid organs (Gonzalez-Garcia et al., Development 120:3033-3042, 1994). Murine BCl-x_{L} protein also binds and is inhibited by low micromolar concentrations of 2-OMeA (Tzung et al., Nat. Cell. Biol. 3:183-191, 2001; Kim et al., Biochemistry 40:4911-4922, 2001). Nonetheless, there was little evidence of damage to normal tissues in mice treated with 2-OMeA₁ at doses that caused substantial apoptotic death and macroscopic regression of human myeloma xenografts. The increased susceptibility of tumor cells to 2-OMeA may have been due to the high levels of Bcl-x_{L} or Bcl-2 present in many cancers. As previously demonstrated, the apoptotic response of transfected hepatocyte cell lines to 2-OMeA was increased with higher cell BCl-x_{L} levels. This paradoxical effect represents a “gain of function” associated with inhibition of the BCl-x_{L}-associated pore activity in vitro. Preferential killing of cells with “high” levels of BCl-x_{L} might afford a desirable therapeutic window for cancer therapy with 2-OMeA.

The three 2-OMeA-sensitive human myeloma cell lines (RPMI-8226, U266, NCI H929) express BCl-x_{L}, as do the leukemia and lymphoma cell lines that were most sensitive to 2-OMeA (Tu et al., Cancer Res. 58:256-262, 1998; Catlett-Falcone et al., Immunity 10: 105-115, 1999; Yanase et al., J. Interferon Cytokine Res. 18:855-861, 1998; Alam et al., Eur. J. Immunol. 27:3485-3491, 1997; Tagami et al., Oncogene 19:5736-5746, 2000; Campos et al., Leuk Lymphoma 33:499-509, 1999). Bcl-x_{L} expression in multiple myeloma has been reported to correlate with disease severity and chemoresistance (Tu et al., Cancer Res. 58:256-262, 1998). However, BCl-x_{L} is also expressed prominently in some of the cell lines resistant to 2-OMeA (e.g., K562). Antimycin A binds to Bcl-2, and may also bind other related anti-apoptotic proteins with conserved hydrophobic clefts (Kim et al., Biochemistry 40:4911-4922, 2001).

The in vivo response of human myeloma cells to 2-OMe antimycin A demonstrated that endogenous Bcl-2-associated mechanisms of tumor cell survival/drug resistance were viable targets for the treatment of multi-drug resistant cancers and, further, that such pathways can be inhibited without causing significant toxicity. The in vitro findings of improved myeloma cell death when 2-OMe antimycin A when combined with standard myeloma chemotherapeutics further supported the targeting of Bcl-2-associated survival mechanisms for new anti-tumor therapies.

### Example 21

Characterization of Bcl-x_{L} Mutant Proteins

Bclx_{L} mutants were derived from pSFFV-Bcl-x_{L}-WT (Example 1) using site directed mutagenesis (QuikChange XL, Stratagene). Briefly, mutagenic primers spanning each target site were used to amplify fragments containing the desired mutations. Residual wild-type template was then removed by digesting with the methylation-dependent endonuclease, DpnI. For recombinant expression, Bcl-x_{L}CΔ22 lacking the COOH-terminal membrane anchor sequence was generated by PCR. PCR products were digested with NdeI and XhoI and ligated into pET22b(+) (Novagen). All constructs were confirmed by sequence analysis.

Staurosporine (STS) and Antimycin Toxicity in TAMH Cells Over-Expressing Bcl-x_{L} Mutants.

TAMH cells were transfected with DNA encoding each of the mutant BCl-x_{L} proteins by lipofection (See Example 1 for method). For analysis of BCl-x_{L} expression, 20 μg of cell protein was separated by 20% SDS-PAGE and transferred to nitrocellulose membranes. Immunodetection of BCl-x_{L} was carried out using rabbit anti-Bcl-x_{L} antibody and Protein A/horseradish peroxidase conjugate, followed by chemiluminescent detection. Cells were grown to about 80% confluence in 96-well plates followed by addition of 100 μl of 2X AA₁ or staurosporine (STS) solution in complete medium. STS is a natural product originally isolated from the bacterium Streptomyces and found to be capable of inducing apoptosis in certain cells and which induction of apoptosis could be blocked by the expression of a Bcl-2 family protein. Cell viability was determined spectrophotometrically after 24 h treatment as the ratio of reduced and oxidized Alamar Blue (BIOSOURCE) at 570 nm and 600 nm, respectively. All results were normalized against DMSO controls. LD₅₀ values were calculated by non-linear regression analysis using Prism software (Graphpad). Results are shown in FIG. 1. Similar results were obtained using sulforhodamine B assays for total cell protein.

Recombinant BCl-x_{L} purification

A pET22b (Novagen) vector coding for BCl-x_{L}(ΔC), a mutant Bcl-x_{L} protein without the COOH terminal region, was transformed into Escherichia coli BL21(DE3) cells that carried pUB520 (encoding human Arg tRNA) and grown to an A₆₀₀ of 0.6. Protein expression was induced with 0.1 mM isopropyl β-D-thiogalactoside at 30° C. The cells were resuspended 1:5 (w/v) in PEB buffer (50 mM Tris, pH 8.0, 200 mM NaCl, 0.2 mM phenylmethylsulfonyl fluoride, 5 mM β-mercaptoethanol, 5 mM imidazole, and 1% (v/v) glycerol), and stirred for 20 min at 4° C. Cells were disrupted by pulse sonication, and the soluble fraction was loaded onto a nickel-nitrilotriacetic acid column (Qiagen) equilibrated with PEB buffer. The column was washed with 40 mM imidazole, eluted with 200 mM imidazole, and the protein fractions were pooled and dialyzed (50 mM Tris, pH 8.0, 200 mM NaCl, 0.2 mM phenylmethylsulfonyl fluoride) at 4° C. overnight. The dialyzed protein was concentrated to 10 mg/ml and fractionated on a Superdex 75 gel filtration column (Amersham Biosciences). The fractions containing Bcl-x_{L}(ΔC) protein were pooled, exchanged into low-salt buffer (same as for previous dialysis buffer, except 50 mM NaCI), and loaded onto a MonoQ anion exchange column (Amersham Biosciences). Protein was eluted from the column with increasing NaCl gradient, pooled and concentrated. Purity was >99% as determined by silver staining, Bcl-x_{L}(ΔC) protein concentrations in 6 M guanidine HCl were determined from 280 nm absorbance using extinction coefficients of 41940 M⁻¹cm⁻¹ for WT, E92L, A142L, and F146L, and 47630 m⁻¹cm⁻¹ for F97W Bcl-x_{L}(ΔC) proteins.

Binding of Antimycin A1 to Wild-Type and Mutant Bcl-x_{L} Proteins

Fluorescence anisotropies of AA₁ and FITC-labeled BAK BH3 peptide were measured using a Fluoromax-3 spectrometer equipped with autopolarizers. All reagents were prepared in 0.2 μm-filtered PBS with fresh 1 mM DTT. Excitation and emission wavelengths were 340 nm and 420 nm for AA₁, and 480 nm and 520 nm for FITC-labeled BAK BH3 peptide respectively. Slit widths were set at 10 nm for both excitation and emission. AA₁ (200 nM) or BH3 peptide (25 nM) were equilibrated with different concentrations of BCl-x_{L}(CΔ22) and mutant BCl-x_{L} proteins for at least 1 h at room temperature. Each data point represents the mean of three independent measurements. Fluorescence anisotropy values were converted to fraction of ligand bound (f_{B}) and expressed on a semi-log plot with non-linear curve fitting. (Lakowicz, Principles of Fluorescence Spectroscopy, 2nd Ed., pp 308-309, Kluwer Academic/Plenum Publishers, New York).

Antimycin Inhibition of Pore-Forming Activity of Wild-Type and Mutant BCl-x_{L} Proteins

Large unilamellar vesicles composed of 60% dioleoylphophatidylcholine and 40% dioleoylphosphatidylglycerol were prepared by the extrusion method of Mayer et al., supra. Lipid stocks, in chloroform, were mixed and dried under a stream of nitrogen gas. The lipid was resuspended by vortexing for 30 min in a solution of 40 mM calcein (Molecular Probes), 25 mM KCl, and 10 mM KOAc, pH 5.0. After 10 freeze-thaw cycles, the lipid suspension was extruded through two 0.1 μm pore diameter Nucleopore filters. Non-entrapped dye was removed by passage over a Sephadex G10 column (Amersham Biosciences). Lipid concentration was measured using the ammonium ferrothiocyanate method (Stewart, Anal. Biochem. 104:10-14, 1980).

For pore assays, recombinant truncated Bcl-2 family protein or mutant protein (Bcl-x_{L}(ΔC)) (500 nM) was added to large unilamellar vesicles (60 μM lipid concentration) in 100 mM KCl, 10 mM KOAc, pH 5.0, and fluorescence measured (490 nm excitation, 520 nm emission) with a Fluoromax-3 spectrophotometer. Peptides of the BH3 domain were incubated with the BCl-x_{L}(ΔC) 5 min prior to mixing with liposomes. AA₁ was added to liposomes 1 min before adding the Bcl-x_{L}(ΔC) protein. Samples for kinetic assays were analyzed in a thermostatted cuvette at 37° C. with constant stirring. Dose responses were measured in black quartz microplates (Hellma) at room temperature. Calcein release was expressed as percentage of maximum release with detergent lysis (0.1% Triton X-100). Pore inhibition was calculated using cumulative dye release normalized to results obtained in absence of inhibitors, and the IC₅₀ values were determined by non-linear regression analysis. Results are shown in Table 9.

Crystallographic Studies

Purified wild-type and mutant BCl-x_{L}(ΔC) proteins were concentrated to 1 mM and crystallized by hanging drop vapor diffusion at 4° C. The mother liquor consisted of 50 mM MES, pH 6.0., 1.9 M ammonium sulfate. Crystals were flash-frozen in liquid nitrogen after soaking in mother liquor plus 30% trehalose (Sigma) for 1 min. Data sets were collected at 100 K with a Rigaku x-ray generator (100 mA and 50 kV) and a Raxis IV imageplate. DENZO and SCALE-PACK (Otwinowski and Minor, in Macromolecular Crystallography (Charles, et al. eds.), pp. 307-326, Academic Press, San Diego, Calif., 1997) were used to process the diffraction data.

The program EPMR (Kissinger et al., Biol. Crystall. D 55:484-491, 1999) was used to find a molecular replacement solution using the Bcl-x_{L}^{wt} structure (Protein Data Bank code 1MAZ) as a starting model. The space group for BCl-x_{L} and all mutant proteins was determined to be P4₁,2₁,2₁. A free R set (Bruger, Nature 355:472-475, 1992) of 10% was set aside using the CCP4 program FreeRflag. The Xfit 4.0 program from the Xtalview suite (McRee, J. Struct. Biol. 125:156-165, 1999) was used to visualize and modify the structure. The CNS (Brunger et al., Biol. Crystall. D 54:905-921, 1998) program package was used for model refinement and simulated annealing composite omit 2FₒF_{c} maps were used to guide model rebuilding. The stereochemical properties of all structures were examined by PROCHECK (Laskowski et al., J. Appl. Cryst. 26:283-291, 1997). Subsequent structural alignments, analysis, and figures were done with Swiss PDB viewer (Guex and Peitsch, Electrophoresis 18:2714-2723, 1997), with pictures rendered using POVRay (available at the website for Persistence of Vision Raytracer, Pty. Ltd.). A summary of crystallographic statistics is provided in Table 9.

TABLE 9
Summary of Crystallographic Statistics.

| Data Statistics | | WT | E92L | F97W | A142L | F146L |
|---|---|---|---|---|---|---|
| Unit Cell | (a) | 63.34 | 63.39 | 64.39 | 63.3 | 63.19 |
| | (b) | 63.34 | 63.89 | 64.39 | 63.3 | 63.19 |
| | (c) | 109.82 | 109.29 | 110.98 | 110.14 | 109.87 |
| Space Group | | P4₁2₁2 | P4₁2₁2 | P4₁2₁2 | P4₁2₁2 | P4₁2₁2 |
| Resolution (Å) | | 1.95 | 2.1 | 2.7 | 2.2 | 2.0 |
| Completeness (%) | | 94.3 | 99.9 | 95.9 | 98.4 | 98.9 |
| R_{merge} (%) | | 3.3 | 3.6 | 7.7 | 7.0 | 4.0 |
| Refined Statistics | | | | | | |
| R_{cryst} | | 20.3 | 21.2 | 19.1 | 20.5 | 20.8 |
| R_{free} | | 21.6 | 23.9 | 23.0 | 23.4 | 20.8 |
| Test Size (%) | | 10 | 10 | 10 | 10 | 10 |
| No. Mol. in Asym Unit | | 1 | 1 | 1 | 1 | 1 |
| No. of non-hydrogen atoms | | | | | | |
| Protein | | 1154 | 1154 | 1156 | 1158 | 1155 |
| Water | | 235 | 143 | 16 | 100 | 222 |
| RMSD from ideal values | | | | | | |
| Bond lengths (Å) | | 0.0075 | 0.0070 | 0.0070 | 0.0054 | 0.0072 |
| Bond angles (°) | | 1.067 | 1.186 | 1.179 | 1.104 | 1.203 |
| Ramachandran plot (%) | | | | | | |
| Most favored regions | | 96.9 | 94.5 | 89.0 | 95.3 | 92.1 |
| Additional allowed regions | | 3.1 | 4.7 | 10.2 | 3.9 | 7.9 |
| Generously allowed regions | | 0 | 0.8 | 0.8 | 0.8 | 0 |
| Disallowed regions | | 0 | 0 | 0 | 0 | 0 |

A series of point mutations were introduced to alter specific residues in the BCL-x_{L} hydrophobic groove contact with AA from the docking model. The following single amino acid substitutions were made in human BCl-x_{L}: E92L, F97W, A142L, and F146L. Stable transfectants of TAMH murine hepatocytes for each of the mutated BCl-x_{L} plasmids as well as wild-type BCl-x_{L}. Mutant BCl-x_{L} (Bcl-x_{L}^{mu}) and wild type proteins (Bcl-x_{L}^{wt}) were expressed at similar levels.

To assess the effect of mutations of BCl-x_{L} function, TAMH/Bcl-x_{L}^{mu} cells were tested for survival during STS treatment. Dose-response curves show that each of the BCl-x_{L} mutant proteins produced equivalent levels of protection against STS-induced cell death. LD₅₀ values for STS with cells expressing BCl-x_{L} mutants were not significantly different from BCl-x_{L} wild-type cells (LD₅₀=0.58±0.1 μM). Vector-only control cells expressed low levels of endogenous BCl-x_{L} and were significantly more sensitive to STS (LD₅₀ 0.11±0.01 μM) than any of the BCl-x_{L} or BCl-x_{L} mutants cell lines (p<0.05). (Table 10).

The BCl-x_{L} and BCl-x_{L} mutant cells were next challenged with antimycin A₁. In contrast to the results with STS, antimycin A sensitivity varied substantially among the Bcl-x_{L} mutant cell lines. Compared with BCl-x_{L} wild-type cells (LD₅₀=0.47±μM) the E92L and F97W BCl-x_{L} mutants had reduced sensitivity to antimycin (LD₅₀=1.72±0.3 μM and 5.12±0.9 μM, respectively), whereas the A142L and F146L BCl-x_{L} mutant cells were completely insensitive to antimycin A₁. (Table 10).

AA-insensitive BCl-x_{L} Mutants have Lower Binding Affinity for Antimycin A₁

Recombinant BCl-x_{L} mutant mutants (Bcl-x_{L}^{mu}(ΔC)) and wild-type proteins were purified from bacterial extracts by nickel-nitrilitriacetic acid affinity gel filtration, and anion-exchange column chromatography. Direct quantitative measurements of AA₁ binding to Bcl-x_{L}(ΔC) proteins were obtained from fluorescent anisotropy under equilibrium conditions. Binding constants were calculated using non-linear regression analysis. (See Table 9). AA₁ has a much weaker binding affinity with the F97W, A142L and F146L mutants (Kd=17.56±5.2 μM, 41.77±21.4 μM, and 20.04±9.4 μM. respectively) than with Bcl-x_{L}^{wt}(ΔC) protein (2.36±1.41 μM). The binding affinity of AA₁ with the E92L mutant (K_{d}=5.06±0.86 μM) was reduced 2- to 3-fold relative to Bcl-x_{L}^{wt}. Notably, the ranking of in vitro AA₁ binding affinities for the Bcl-x_{L}^{mu}(ΔC) proteins is in register with the in vivo sensitivities to AA.

TABLE 10
Summary of AA₁ activity on wild-type and
mutant Bcl-x_{L} proteins.

| Cytotoxicity | | | Dissociation Constant | | Pore Inhibition | |
|---|---|---|---|---|---|---|
| | STS LD₅₀ | AA₁ LD₅₀ | | BH3 K_{D} | AA₁ IC₅₀ | BAK BH3 |
| Bcl-x_{L} | (μM) | (μM) | AA₁ K_{D} (μM) | (μM) | (μM) | IC₅₀ (μM) |
| WT | 0.58 ± 0.14 | 0.47 ± 0.07 | 2.36 ± 1.41 | 0.11 ± 0.029 | 2.03 ± 0.24 | 0.58 ± 0.36 |
| E92L | 0.52 ± 0.12 | 1.72 ± 0.34² | 5.06 ± 0.86¹ | 0.22 ± 0.05 | 2.47 ± 0.30 | 2.00 ± 0.87 |
| F97W | 0.34 ± 0.10 | 5.12 ± 0.94² | 17.56 ± 5.20² | 2.89 ± 0.41² | 3.45 ± 0.34² | 71.61 ± 9.01² |
| A142L | 0.49 ± 0.11 | >10 | 41.77 ± 21.49¹ | 13.00 ± 5.57¹ | 4.10 ± 1.24¹ | ND |
| F146L | 0.38 ± 0.04 | >10 | 20.04 ± 9.41¹ | 1.26 ± 3.57² | 3.60 ± 0.40² | 10.62 ± 3.15² |

¹indicates p < 0.05
²indicates p < 0.01
ND indicates that the non-linear regression was unable to fit this curve due to a lack of inhibition

The non-peptide BCl-x_{L} inhibitors, BH3I-1 and BH3I-2, interact with Phe-97 and Ala-142 in the BCl-x_{L} hydrophobic pocket by NMR chemical shift perturbation (Degterev et al., Nat. Cell Biol. 3:173-182, 2001; Lugovskoy et al., J. Amer. Chem. Soc. 124:1234-1240, 2002). It was determined that BH3I-1 competes with AA₁ for Bcl-x_{L}(ΔC) binding. The Kᵢ for Bh3-I-1 displacement of AA bound to Bcl-x_{L}(ΔC) was 1.874±0.617 μM, similar to that reported for displacing BH3 peptide (Degterev et al., supra).

The affects of the hydrophobic groove mutations on binding of BAK BH3 domain peptides was also determined. Fluorescent anisotropy measurements were conducted using the FITC-labelled 16-residue BAK-BH3-peptide (SEQ ID NO: 1). The F97W, A142L, and F146L mutations resulted in substantially diminished BH3 peptide binding compared with Bcl-x_{L}^{wt}(ΔC). Interestingly, the relative affinities of BAK BH3 peptide with the Bcl-x_{L}^{mu}(ΔC) proteins (Bcl-x_{L}^{wt}>E92L>F97W˜F146L>A142L) paralleled those determined for AA.

Pore-Forming Activities of Mutant BCl-x_{L} Proteins

Synthetic lipid vesicles were loaded with the self-quenching fluorescent dye, calcein, to measure membrane pore formation b recombinant BCl-x_{L}(ΔC) proteins as previously described. Addition of AA₁, 2-OMeAA₁, or the BAK BH3 peptide inhibited Bcl-x_{L} pore-forming activity, whereas a modified antimycin bearing a bulky phenacyl substituent, a mutated BAK peptide (L78A) with low BCl-x_{L} affinity and BH3I-1 had no effect.

The mutant versions of BCl-x_{L}(ΔC) had similar pore-forming properties as Bcl-x_{L}^{wt}(ΔC). The sensitivity of Bcl-x_{L}^{mu}(ΔC) pores to AA₁ and to BAK BH3 peptide was measured under similar experimental conditions and non-linear regression analysis of the dose-response curves was performed to obtain IC₅₀ values (Table 9). Although the clustering of AA₁ IC₅₀ values in the liposome assay was tighter than observed for the binding affinities of AA₁ using soluble Bcl-x_{L}(ΔC), the same ordering of responses was obtained: WT>E92L>F97W˜F146L A142L. The BAK BH3 peptide IC₅₀ values also exhibited the same ordering observed for the binding affinities obtained from the equilibrium binding assay.

Preservation of Tertiary Fold with Mutant BCl-x_{L} Proteins

The structures of Bcl-x_{L}^{wt}(ΔC) and the four Bcl-x_{L}^{mu}(ΔC) proteins, E92L, F97W, A142L, and F146L, were solved by x-ray crystallography. Overall, the mutations produced only local effects on the wild-type structure. An a-carbon overlay of the wild-type protein with the mutant structures was carried out. The largest differences were primarily localized to the a3 helix between residues Tyr-101 and His-113. The RMSD of the Bcl-x_{L}mutants ranged from 0.17 to 0.48 Å (Cα atoms).

E92L—In the docking model, the backbone carbonyl of Glu-92 contacts the O33 atom of AA₁, whereas the side chain projects outside the hydrophobic groove with no close ligand contacts. The structure of E92L Bcl-x_{L}(ΔC) at 2.1 Å compared with Bcl-x_{L}^{wt}(ΔC) shows no main chain movement and only a minor displacement between the Leu and Glu side chains. The hydrogen bonds bridging Gln-88 and Asn-198 were missing, weakening interactions between the BH3 α-helical domain and the COOH-terminal region of Bcl-x_{L}(ΔC). Overall, the structure was very similar to wild-type, with a Cα RMSD of 0.17 Å, and an all-atom RMSD of 0.40 Å.

F97W—The Phe-97 residue lies in close proximity to the dilactone ring of AA₁ in the docking model. Thus, the greater bulk of a Trp side chain in this position was expected to cause a steric clash with bound AA₁. The structure of F97W Bcl-x_{L}(ΔC) was solved to 2.7 Å. The F97W substitution did not significantly disrupt the backbone structure (overall RMSD Cα of 0.30 Å, F97W RMSD of 0.29 Å). However, to compensate for the bulkier Trp side chain, Phe-101 rotates about χ₂ approximately 80 degrees. The maximal backbone displacement for F97W Bcl-x_{L}(ΔC) occurred in this region, with residues 101 through 106 having an RMSD Cα of 0.67, although this displacement was significantly less than in the structure of a Bcl-x_{L}/BH3 peptide complex (Protein Data Bank code 1BXL). Thus, the steric effects of Trp-97 on AA₁ affinity should be predominant.

A142L—The A142 residue was positioned adjacent to Phe-97 in the hydrophobic groove. The docking model for AA₁ predicted a significant steric clash between the dilactone ring of AA₁ and the Leu-142 side chain. The structure of A142L BCl-x_{L}(ΔC) was solved to 2.2 Å. The A142L BCl-x_{L}(ΔC) structure was very similar to Bcl-x_{L}^{wt}, with a RMSD (Cα) of 0.44 Å. The bulkier leucine side chain caused a compensatory chain of movement of the Phe-97 and Tyr-101 side chains. Furthermore, repositioning of the Tyr-101 backbone promoted alternative orientations of Ala-104 and Phe-105, which flipped from higher energy positive backbone φ/ψ angles to lower energy negative φ/ψ angles. Despite the movement of the Phe-105 main chain, the side chain orientation was conserved with Bcl-x_{L}^{wt}(ΔC). Because Ala-104 and Phe-105 also had negative φ/ψ angles in the BH3 peptide/Bcl-x_{L} structure, the binding pocket in the ligand-bound conformation of A142 Bcl-x_{L}(ΔC) should be preserved without significant structural changes.

F146L—Unlike the other BCl-x_{L} mutations considered here, the docking model predicted a loss of van der Waals contacts to the alkyl chain of AA₁ with the F146L substitution. The F146L BCl-x_{L}(ΔC) structure was solved to 2.2 Å. The F146L BCl-x_{L}(ΔC) structure showed an overall RMSD (Cα) of 0.49 Å. There was little displacement of the F146L residue compared with Bcl-x_{L}^{wt}(ΔC). The aliphatic and aromatic side chains of the α3 helix and neighboring residues to F146L adopted wild-type orientations with the exception of the rotation of Lys-108 about χ₂. As with the A142L Bcl-x_{L}(ΔC) structure, residues Ala-104 and Phe-105 converted from positive to negative φ/ψ angles. In addition, the Arg-103 backbone had adopted a positive φ/ψ configuration in F146L BCl-x_{L}. Notably, the average B-factors across all structures, including Bcl-x_{L}^{wt}(ΔC), for residues 101-105 in the α3 helix were about twice that of the rest of the molecule. Thus, the alterations in backbone configuration at residues 103-105 in the Bcl-x_{L}^{mu} proteins may reflect an inherent flexibility of this region. The F146L BCl-x_{L} structure also demonstrated enlargement of an interior cavity abutted by Phe-146, which may reduce the overall stability of the protein (Baldwin et al., J. Mol. Biol. 259:542-559, 1996; Xu et al., Prot. Sci. 7:158-177, 1998). In Bcl-x_{L}^{wt}(ΔC), this cavity has a calculated area of 34 Å², which increased to 54 Å² in the F146L mutant structure.

In prior examples set forth above it was demonstrated that expression of the anti-apoptotic protein BCl-x_{L} rendered cells hypersensitive to antimycin A. Antimycin A bound directly to BCl-x_{L}(ΔC) in competition with BH3 peptide ligands that occupy the hydrophobic groove, consistent with the identification of this interface as the likely antimycin A binding site by molecular modeling. Site-directed mutagenesis has been used to validate BCl-x_{L} as a direct target for antimycin A and map the BCl-x_{L} binding site for antimycin A in greater detail.

Three out of four mutations in the BCl-x_{L} hydrophobic groove (F97W, A142L, and F146L) eliminated or strongly attenuated the ability of BCl-x_{L} to sensitize TAMH cells to antimycin A₁ treatment. Each of the mutants had nearly wild-type anti-apoptotic activity with staurosporine treatment, discounting loss of protein function as an explanation for the resistance to antimycin A₁. However, it has been demonstrated that reduced binding affinities of antimycin A₁ with the Bcl-x_{L}^{mu}(ΔC) proteins, with a good correlation between binding constant and in vivo sensitivity to antimycin A₁.

The BCl-x_{L} mutations were designed for local perturbations on ligand-protein geometry. The crystal structures of the BCl-x_{L}(ΔC) mutants demonstrated retention of the tertiary protein fold, allowing interpretation of the binding data in terms of the molecular docking model. The docking model for antimycin A₁ utilized the structure of Bcl-x_{L}(ΔC) bound to BAK BH3 peptide. Compared with the overall shift between ligand-bound and free BCl-x_{L} conformations (Cα RMSD=2.8 Å), there was minimal displacement of the residues predicted to be antimycin A₁ contacts in these structures (Cα RMSD for Phe-97, Ala-142, and Phe-146=1.3 Å).

The relative antimycin A₁ binding affinities of the Bcl-x_{L}^{mu}(ΔC) proteins were as follows: WT>E92L>F97W˜F146L>A142L. Incorporating these single mutations into the AA₁ docking model allowed for the prediction of their effects on AA₁ binding. Both F97W and A142L mutations were modeled to produce steric hindrances to the docked AA₁. In the former case, the increased bulkiness of the tryptophan side chain made close contacts (approximately 2.6 Å) to the dilactone ring of AA₁, whereas the A142L substitution created a significant steric clash to the AA₁ dilactone ring. The 8-fold and 20-fold increases in the K_{d} of AA₁ binding for F97W and A142L Bcl-x_{L}^{mu}(ΔC), respectively, compared with Bcl-x_{L}^{wt}(ΔC), were comparable with these predictions. To form a stable complex, significant compensatory movements of the BCl-x_{L} binding pockets or AA₁ from the starting docking model must occur.

The phenyl group at Phe-146 was oriented perpendicularly to the hexyl-chain of AA₁ in the docking model. This predicted interaction consisted of van der Waals contacts and electrostatic interactions between the partial negative charges of the phenyl ring and partial positive charge of the terminal methyl group (C27). Substitution of leucine for phenylalanine removed both types of contacts with AA₁ accounting for the approximate 10-fold decrease in AA₁ affinity with this mutant protein. Overall, the ligand-bound structure models based on the Bcl-x_{L}^{mu}(ΔC) crystal structures provide reasonable interpretations for the measured AA₁ binding constants. The E92L mutation was not expected to alter AA binding based on the docking model, as it was located at the periphery of the binding pocket and contributed only a carbonyl oxygen contact with AA₁. Thus, the 2- to 3-fold reduction of AA₁ binding affinity with the E92L mutation provided an estimate of the general effects on binding for mutations at partially buried residues. The mild reduction in affinity may be due to some destabilization of the BCl-x_{L} tertiary structure by the loss of two salt bridges.

The anti-apoptotic activities of BCl-x_{L} and Bcl-2 act through and are regulated by associations with pro-apoptotic proteins. The Bcl-x_{L}^{mu} proteins retained normal anti-apoptotic activity despite substantially weakened binding to the pro-apoptotic BH3 peptide. BCl-x_{L} binding affinities for BH3 peptides depended on hydrophobic interactions at the floor of the cleft with several conserved non-polar residues (Val-74, Leu-78, and Ile-81) in the peptides. Modeling studies of the BAK BH3 peptide to the BCl-x_{L} mutants suggested that the F146L substitution weakened interactions with BAK Val-74, whereas the F97W mutation needed to be moderately displaced to avoid a clash with the side chain of Bak Leu-78. However, BCl-x_{L} A142L required a much larger adjustment to avoid a clash between the backbone of BAK Leu-78 and the BCl-x_{L} Leu-142 side chain. Overall, these results suggested that the mutant phenotypes embodied here were more compatible with the pro-apoptotic binding partner exerting negative control over the anti-apoptotic function of BCl-x_{L}, rather than vice versa. However, only a single pro-apoptotic BH3 domain (BAK) has been tested. For example, BCl-x_{L} and Bcl-2 proteins with mutations preventing binding to BAK nevertheless strongly interacted with the BH3 only BAD protein in a previous report (Ottillie et al., J. Biol. Chem. 272:272:20866-20872, 1997).

The hydrophobic groove mutations did not affect the pore-forming ability of purified BCl-x_{L}(ΔC) in synthetic liposomes, suggesting that this property was endowed by the global protein fold and packing geometry of BCl-x_{L}(ΔC). AA₁ inhibited pore formation of mutant and wild-type proteins with similar IC₅₀ values. The insensitivity of this assay for discriminating mutants with different AA binding affinities implied AA₁ interacts differently with soluble versus membrane-inserted BCl-x_{L}(ΔC). Using dansylated lipid in fluorescence resonance energy transfer experiments, it was determined that AA₁ does not interfere with the insertion of BCl-x_{L} into lipid membranes whereas BAK BH3 inhibits full membrane insertion of BCl-x_{L}. This finding explained why the distribution of BAK BH3 IC₅₀ values for the Bcl-x_{L} mutant series reflected the range of affinities for soluble BCl-x_{L} protein, because pore inhibition took place at a pre-insertion step. The conserved ordering of mutant protein activities in AA binding and pore assays (i.e., WT>E92L>F97W˜F146L>A142L) argued for similarities in the soluble and membrane-inserted binding interfaces. The AA₁-BCl-x_{L}(ΔC) interaction in a lipid environment appeared to be significantly less constrained by the BCl-x_{L} mutations considered herein compared with the soluble form of the protein.

The mutational study results in this example strongly support the earlier conclusion set forth above that AA selectively kills Bcl-x_{L} expressing cells by directly targeting BCl-x_{L}. The single amino acid mutations produced minor shifts in the binding pocket geometry, allowing a high degree of confidence in extrapolating from crystal structures to the ligand-bound protein conformation. The principal basis for the antimycin A resistance of the mutant BCl-x_{L} proteins appeared to be lower binding affinity, as reflected by the strong correlation between in vitro binding constants and cytotoxicity. In aggregate, these results agreed with the starting molecular model of how AA₁ bound to the BCl-x_{L} hydrophobic groove.

Although the foregoing invention has been described in some detail by way of illustration and example for purposes of clarity of understanding, it will be obvious that certain changes and modifications may be practiced within the scope of the appended claims. The scope of the invention should, therefore, be determined not with reference to the above description, but instead should be determined with reference to the appended claims along with their full scope of equivalents. All publications and patent documents cited in this application are incorporated by reference in their entirety for all purposes to the same extent as if each individual publication or patent document were so individually denoted.

1. A 2-methoxy derivative or analog of antimycin that modulates apoptosis by binding to a Bcl-2 family member protein and having the structural formula represented by Formula (II) 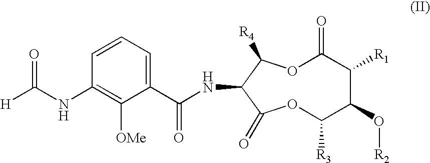
wherein
R₁ is a hydrogen, a C₁-C₈ linear or branched alkane, a hydroxyl, a C₁-C₈ hydroxyalkane, an amino, a C₁-C₈ di- or tri-amine, a C₁-C₈ amide, a C₁-C₈ carboxylic acid, or a substituted alkyl group;
R₂ is a C₁-C₈ acyl group;
R₃ is a hydrogen, a C₁-C₈ linear or branched alkane, a hydroxyl, a C₁-C₈ hydroxyalkane, an amino, a C₁-C₈ di- or tri-amine, a C₁-C₈ amide, a C₁-C₈ carboxylic acid, or a substituted alkyl group; and
R₄ is a hydrogen, a C₁-C₈ linear or branched alkane, a hydroxyl, a C₁-C₈ hydroxyalkane, an amino, a C₁-C₈ di- or tri-amine, a C₁-C₈ amide, a C₁-C₈ carboxylic acid, or a substituted alkyl group,
with the proviso that the 2-methoxy antimycin derivative is not 2-methoxy antimycin A₃.

2. The 2-methoxy antimycin derivative or analog of claim 1, wherein R₁ is a C₁-C₈ linear alkane.

3. The 2-methoxy antimycin derivative or analog of claim 2, wherein R₁ is selected from the group consisting of an ethyl group, a butyl group, and a hexyl group.

4. The 2-methoxy antimycin derivative or analog of claim 1, wherein R₄ is a methyl group.

5. The 2-methoxy antimycin derivative or analog of claim 1, wherein R₂ has the structural formula represented by Formula (III) 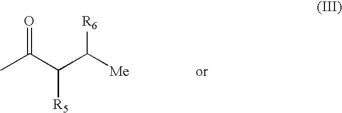
Formula (IV), 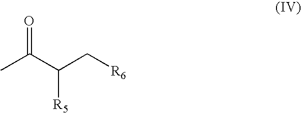
wherein R₅ and R₆ are each independently selected from the group consisting of a methyl group and a hydrogen.

6. The 2-methoxy antimycin derivative or analog of claim 1, which has the structural formula represented by Formula (V)

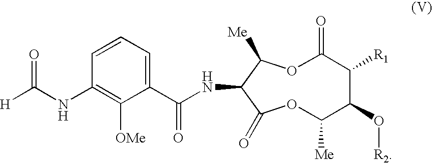

7. The 2-methoxy antimycin A derivative or analog of claim 6, wherein R₁ is a C₁-C₈ linear alkane.

8. The 2-methoxy antimycin derivative or analog of claim 7, wherein R₁ is selected from the group consisting of an ethyl group, a butyl group, and a hexyl group.

9. The 2-methoxy antimycin derivative or analog of claim 6, wherein R₂ has the structure represented by Formula (III) 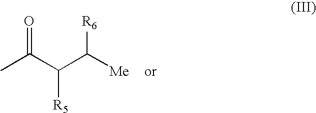
Formula (IV), 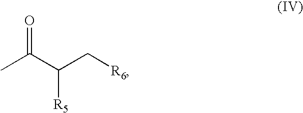
wherein R₅ and R₆ are each independently selected from the group consisting of a methyl group and a hydrogen.

10. The 2-methoxy antimycin derivative or analog of claim 6, which has the structural formula represented by Formula (VI)

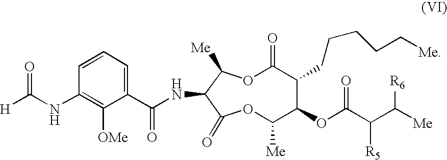

11. The 2-methoxy antimycin derivative or analog of claim 6, which has the structural formula represented by Formula (VII)

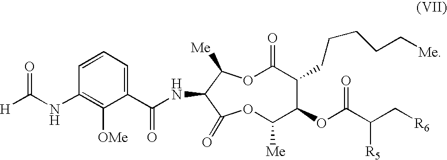

12. The 2-methoxy antimycin derivative or analog of claim 6, which has the structural formula represented by Formula (VIII)

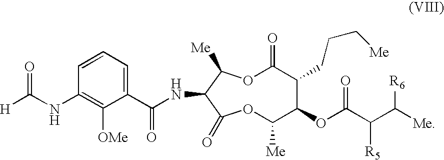

13. The 2-methoxy antimycin-derivative or analog of claim 6, which has the structural formula represented by Formula (IX)

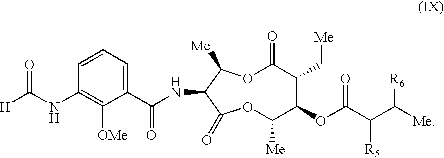

14. The 2-methoxy antimycin derivative or analog of claim 1, wherein a lactone oxygen has been replaced by nitrogen.

15. The 2-methoxy antimycin derivative or analog of claim 1, wherein the ester oxygen has been replaced by nitrogen.

16. The w-methoxy antimycin derivative or analog of claim 1, wherein the Bcl-2 family member protein is Bcl-2 or BCl-x_{L}.

17. The 2-methoxy antimycin derivative or analog of claim 1, which has a reduced binding affinity for BCl-x_{L} protein having a mutation selected from the group consisting of E92L, F97W, L130A, A142L, F146L, and Y195G, said reduced binding affinity relative to the binding affinity for wild-type BCl-x_{L}.

18. The 2-methoxy antimycin derivative or analog of claim 1, further comprising a pharmaceutically acceptable carrier.

19. The 2-methoxy antimycin derivative or analog of claim 1, further comprising an inhibitor of esterase activity.

20. A pharmaceutical composition for treatment of an apoptosis-associated disease in a subject, the composition comprising the 2-methoxy antimycin derivative or analog of claim 1.

21. The pharmaceutical composition of claim 20, further comprising an inhibitor of esterase activity.

22. A method for treating an apoptosis-associated disease in a subject, the method comprising administering to the subject a therapeutically effective amount of a 2-methoxy antimycin derivative or analog that modulates apoptosis by binding to a Bcl-2 family member protein and having the structural formula represented by Formula (I) 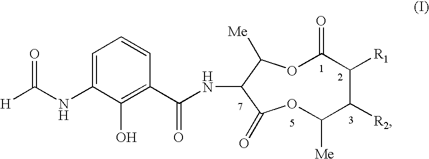
wherein
R₁ is a hydrogen, a C₁-C₈ linear or branched alkane, a hydroxyl, a C₁-C₈ hydroxyalkane, an amino, a C₁-C₈ di- or tri-amine, a C₁-C₈ amide, a C₁-C₈ carboxylic acid, or a substituted alkyl group;
R₂ is a C₁-C₈ acyl group;
R₃ is a hydrogen, a C₁-C₈ linear or branched alkane, a hydroxyl, a C₁-C₈ hydroxyalkane, an amino, a C₁-C₈ di- or tri-amine, a C₁-C₈ amide, a C₁-C₈ carboxylic acid, or a substituted alkyl group; and
R₄ is a hydrogen, a C₁-C₈ linear or branched alkane, a hydroxyl, a C₁-C₈ hydroxyalkane, an amino, a C₁-C₈ di- or tri-amine, a C₁-C₈ amide, a C₁-C₈ carboxylic acid, or a substituted alkyl group.

23. The method of claim 22, wherein R₁ is a C₁-C₈ linear alkane.

24. The method of claim 23, wherein R₁ is selected from the group consisting of an ethyl group, a butyl group, and a hexyl group.

25. The method of claim 22, wherein R₄ is a methyl group.

26. The method of claim 22, wherein R₂ is a methyl group.

27. The method of claim 22, wherein R₂ has the structural formula represented by Formula (III) 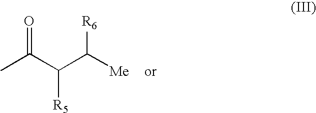
Formula (IV), 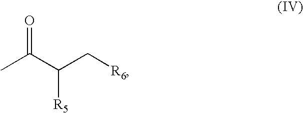
wherein R₅ and R₆ are each independently selected from the group consisting of a methyl group and a hydrogen.

28. The method of claim 22, which has the structural formula represented by Formula (V)

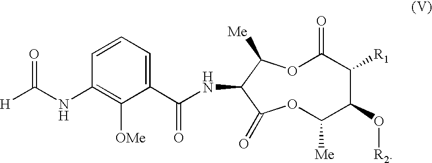

29. The method of claim 28, wherein R₁ is a C₁-C₈ linear alkane.

30. The method of claim 29, wherein R₁ is selected from the group consisting of an ethyl group, a butyl group, and a hexyl group.

31. The method of claim 28, wherein R₂ has the structural formula represented by Formula (III) 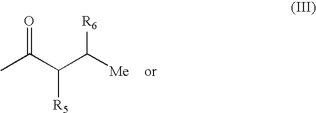
Formula (IV), 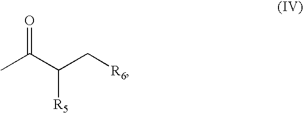
wherein R₅ and R₆ are each independently selected from the group consisting of a methyl group and a hydrogen.

32. The method of claim 28, wherein the 2-methoxy antimycin derivative has the structural formula represented by Formula (VI)

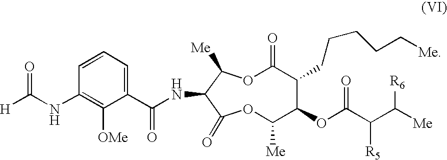

33. The method of claim 28, wherein the 2-methoxy antimycin derivative has the structural formula represented by Formula (VII)

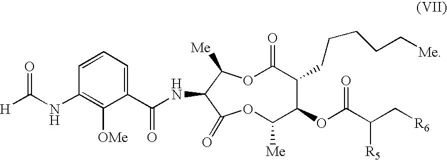

34. The method of claim 28, wherein the 2-methoxy antimycin derivative has the structural formula represented by Formula (VIII)

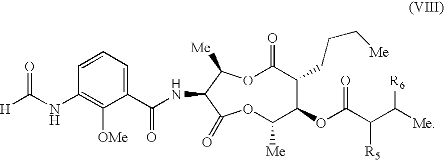

35. The method of claim 28, wherein the 2-methoxy antimycin derivative has the structural formula represented by Formula (IX)

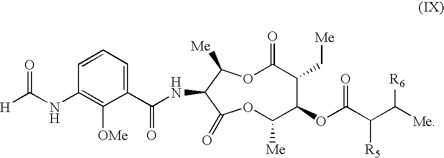

36. The method of claim 22, wherein the Bcl-2 family member protein is Bcl-2 or BCl-x_{L}.

37. The method of claim 22, wherein the 2-methoxy antimycin derivative or analog has a reduced binding affinity for BCl-X_{L} protein having a mutation selected from the group consisting of F92L, F97W, L130A, A142L, F146L, and Y195G, said reduced binding affinity relative to the binding affinity for wild-type BCl-X_{L}.

38. The method of claim 22, wherein the apoptosis-associated disease is a neoplastic disease.

39. The method of claim 38, wherein the neoplastic disease is a cancer.

40. The method of claim 39, wherein the cancer comprises a solid tumor.
